(19) United States
(12) Patent Application Publication
(10) Pub. No.: US 2026/0240983 A1
(43) Pub. Date: Aug. 20, 2026

(54) AVIAN METAPNEUMOVIRUS ISOLATES AND IMMUNOGENIC COMPOSITIONS THEREFROM

(71) Applicant: Iowa State University Research Foundation, Inc., Ames, IA (US)

(72) Inventors: Jianqiang Zhang, Ames, IA (US); Liying Tian, Ames, IA (US); Phillip Gauger, Ames, IA (US); Mohamed El-Gazzar, Ames, IA (US); Yuko Sato, Ames, IA (US)

(21) Appl. No.: 19/545,437

(22) Filed: Feb. 20, 2026

Related U.S. Application Data
(60) Provisional application No. 63/760,980, filed on Feb. 20, 2025.

(51) Int. Cl. A61K 39/155; A61K 39/00; C12Q 1/686; C12Q 1/70

(52) U.S. Cl. CPC A61K 39/155; C12Q 1/686; C12Q 1/70; A61K 2039/51; A61K 2039/5252; A61K 2039/5254; A61K 2039/552; A61K 2039/70; C12N 2760/18334; C12N 2760/18351; C12N 2760/18364; C12Q 2600/106

(57) ABSTRACT

The present disclosure is directed to novel avian metapneumovirus (aMPV) isolates, including aMPV serially propagated in cell culture, all of which are useful in the preparation of immunogenic compositions and vaccines for treating and preventing disease in birds.

### CROSS REFERENCE TO RELATED APPLICATIONS

This application claims priority to provisional application U.S. Ser. No. 63/760,980, filed Feb. 20, 2025, which is incorporated herein by reference in its entirety.

### SEQUENCE LISTING XML

The instant application contains a sequence listing, which has been submitted in XML file format by electronic submission and is hereby incorporated by reference in its entirety. The XML file, created on Feb. 17, 2026, is named P15127US01.xml and is 237,704 bytes in size.

### TECHNICAL FIELD

This disclosure relates generally to the field of vaccines, particularly to novel isolates of avian metapneumovirus (aMPV) and immunogenic compositions that protect birds from disease caused by aMPV.

### BACKGROUND

Avian metapneumovirus (aMPV), previously referred to as avian pneumovirus or avian rhinotracheitis virus or turkey rhinotracheitis virus, can infect various bird species. However, it primarily affects turkeys and chickens, causing respiratory tract infections characterized by sinusitis, swollen heads, increased mortality, and reduced egg production, leading to significant economic losses. Avian metapneumovirus is currently classified into four subtypes (aMPV-A, -B, -C, and -D). Avian metapneumovirus is known to be particularly difficult to isolate due to the fact that excretion of the virus from infected tissue happens to be for a limited time post infection, as well as its instability in diagnostic materials. For this reason, very few laboratories have been successful in isolation and growth of aMPV-A and aMPV-B in cell culture, especially in continuous cell lines, which has limited the characterization of the viruses and development of vaccines.

### SUMMARY

Avian metapneumovirus (aMPV) subtypes A and B were first detected in U.S. poultry in late 2023 and early 2024, rapidly spreading nationwide and posing a significant threat to the poultry industry. aMPV-A and aMPV-B strains associated with this disease outbreak were successfully isolated in primary chicken embryo cells and adapted to a Vero cell line. These U.S. aMPV-A and aMPV-B cell culture isolates provide valuable tools for studying pathogenesis, determining virus infectious doses, evaluating disinfectants and antivirals, and developing vaccines.

The present disclosure encompasses immunogenic compositions comprising aMPV strains. The aMPV strains may be used, in certain embodiments, as inactivated or live attenuated vaccines. Thus, the disclosure comprises an immunogenic composition, suitable to be used as a vaccine, which comprises an aMPV strain of the disclosure, preferably live and attenuated, or an immunogenic fragment thereof, one or more adjuvants, and optionally one or more excipients, in an amount effective to elicit production of neutralizing antibodies in an avian subject.

The immunogenic compositions of the disclosure protect avian subjects from infection by aMPV. The present disclosure includes novel nucleotide and amino acid sequences of aMPV, including novel genotypes thereof, all of which are useful in the preparation of vaccines for treating and preventing diseases in avian subjects including chickens and turkeys. Diagnostic and therapeutic polyclonal and monoclonal antibodies are also a feature of the present disclosure, as are infectious clones useful in the propagation of the virus and in the preparation of vaccines. The disclosure also provides the full-length genomic sequences of aMPV strains at different passages in cell culture.

The present disclosure provides methods for inducing an immune response against aMPV and methods of treating or preventing a disease in an avian subject caused by infection with aMPV, including disease states that are directly caused by aMPV, and disease states contributed to or potentiated by aMPV. The present disclosure also includes the option to administer a combination vaccine, that is, a bivalent or multivalent combination of antigens, which may include live, modified live, or inactivated antigens against a non-aMPV pathogen, with appropriate choice of adjuvant. The present disclosure also provides methods for determining if a population of avian subjects is in need of vaccination against aMPV infection.

Representative embodiments of the disclosure include an isolated polynucleotide sequence that includes a genomic polynucleotide which encodes aMPV proteins which are attenuated and may be used as an immunogenic composition. This can include whole genome sequences selected from:
(a) SEQ ID NO: 1, 2, 3, 4, 5, 6, 7, 8, 9, 10, 11, 12, 13, 14, 15, or 16 or an immunogenic fragment thereof;
(b) the complement of any sequence in (a);
(c) a polynucleotide that hybridizes with a sequence of (a) or (b) under stringent conditions defined as hybridizing to filter bound DNA in 0.5M NaHPO₄, 7% SDS, 1 mM EDTA at 65° C., and washing in 0.1×SSC/0.1% SDS at 68° C.;
(d) a polynucleotide that is at least 70% identical to the polynucleotide of (a) or (b);
(e) a polynucleotide that is at least 80% identical to the polynucleotide of (a) or (b);
(f) a polynucleotide that is at least 90% identical to the polynucleotide of (a) or (b);
(g) a polynucleotide that is at least 95% identical to the polynucleotide of (a) or (b);
(h) a polynucleotide that is at least 98% identical to the polynucleotide of (a) or (b); and
(i) a polynucleotide that is at least 99% identical to the polynucleotide of (a) or (b).

The disclosure further provides RNA and DNA molecules, their complements, fragments and vectors and plasmids for the expression of any such RNA or DNA polynucleotides, and for aMPV that is expressed from such nucleotide sequences, wherein said virus is live, or fully or partially attenuated.

Methods of preparing a live attenuated aMPV such that the virus fails to cause clinical signs of aMPV when administered to an avian subject but is capable of inducing an immune response that immunizes the avian subject against pathogenic forms of aMPV are also provided.

The disclosure also provides a vaccine that comprises a polynucleotide sequence as aforementioned, and corresponding nucleotide sequences that may function as infectious clones.

The disclosure further provides nucleic acid sequences and resultant protein variants that have amino acid substitutions, and which reduce virulence, cause attenuation and allow the compositions to be used safely as immunogenic compositions and as vaccines. In certain embodiments, the nucleic acid and protein sequences include at least one base or amino acid change such that the sequence is not a naturally occurring sequence.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE DRAWINGS

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1A-F shows cytopathic effect (CPE) images of Vero cell-adapted avian metapneumovirus (aMPV) subtypes A and B isolates with the passage 5 (P5) in Vero cells. Images at 40 hours post infection (FIGS. 1A-C) and 60 hours post infection (FIG. 1D-F) are shown.

FIG. 2A-I shows immunofluorescence staining of mock- or virus-infected cells. Vero cells inoculated with culture medium (mock), aMPV-A isolate USA/IA55601-6/2024, or aMPV-B isolate USA/NC23734-GA/2024 were fixed at 24 hours post inoculation. The cells were stained with aMPV antibody-negative turkey serum (FIG. 2A-C), aMPV-A antibody-positive turkey serum (FIGS. 2D-F), and aMPV-B antibody-positive turkey serum (FIG. 2G-I), followed by staining with goat anti-turkey IgG conjugated to fluorescein isothiocyanate (FITC).

FIG. 3A-B shows phylogenetic trees based on the whole genome sequences (FIG. 3A) and the G gene nucleotide sequences (FIG. 3B) of aMPV-A and aMPV-B sequences from this study together with 46 aMPV sequences retrieved from GenBank (13 aMPV-A, 18 aMPV-B, 14 aMPV-C, and one aMPV-D sequences). The aMPV-A isolates and the aMPV-B isolates characterized at different passages in cell culture in this study are depicted using solid gray circles and solid black circles, respectively.

### BRIEF DESCRIPTION OF THE SEQUENCES

SEQ ID NO: 1 is the aMPV-A USA/IA55601-6/2024 primary cells P1 genome sequence. SEQ ID NO: 2 is the aMPV-A/turkey/USA/IA55601-6/2024 primary cells P9 genome sequence. SEQ ID NO: 3 is the aMPV-A/turkey/USA/IA55601-6/2024 Vero cells P4 genome sequence. SEQ ID NO: 4 is the aMPV-A/turkey/USA/IA55601-6/2024 Vero cells P10 genome sequence. SEQ ID NO: 5 is the aMPV-A/turkey/USA/IA56509-5/2024 primary cells P3 genome sequence. SEQ ID NO: 6 is the aMPV-A/turkey/USA/IA56509-5/2024 primary cells P9 genome sequence. SEQ ID NO: 7 is the aMPV-A/turkey/USA/IA56509-5/2024 Vero cells P4 genome sequence. SEQ ID NO: 8 is the aMPV-A/turkey/USA/IA56509-5/2024 Vero cells P10 genome sequence. SEQ ID NO: 9 is the aMPV-B/turkey/USA/NC20487-GA/2024 primary cells P3 genome sequence. SEQ ID NO: 10 is the aMPV-B/turkey/USA/NC20487-GA/2024 primary cells P9 genome sequence. SEQ ID NO: 11 is the aMPV-B/turkey/USA/NC20487-GA/2024 Vero cells P4 genome sequence. SEQ ID NO: 12 is the aMPV-B/turkey/USA/NC20487-GA/2024 Vero cells P10 genome sequence. SEQ ID NO: 13 is the aMPV-B/turkey/USA/NC23734-GA/2024 primary cells P1 genome sequence. SEQ ID NO: 14 is the aMPV-B/turkey/USA/NC23734-GA/2024 primary cells P9 genome sequence. SEQ ID NO: 15 is the aMPV-B/turkey/USA/NC23734-GA/2024 Vero cells P4 genome sequence. SEQ ID NO: 16 is the aMPV-B/turkey/USA/NC23734-GA/2024 Vero cells P10 genome sequence. SEQ ID NO: 17 is a Phosphoprotein (P) amino acid sequence. SEQ ID NO: 18 is a Matrix protein (M) amino acid sequence. SEQ ID NO: 19 is a Fusion protein (F) amino acid sequence. SEQ ID NO: 20 is a M2.2 protein amino acid sequence. SEQ ID NO: 21 is a surface glycoprotein (G) amino acid sequence. SEQ ID NO: 22 is a large polymerase (L) amino acid sequence.

### DETAILED DESCRIPTION

Avian metapneumovirus is a member of the genus Metapneumovirus in the family Pneumoviridae. The aMPV comprises a single-stranded, non-segmented, and negative-sense RNA genome with a length of ~13.3-14 kb, which contains nine genes in the order of 3′-N-P-M-F-M2.1-M2.2-SH-G-L-5′ with untranslated regions (UTR) on both ends (2). The corresponding proteins encoded by these genes are nucleoprotein (N), phosphoprotein (P), matrix protein (M), fusion protein (F), second matrix proteins (M2.1 and M2.2), small hydrophobic protein (SH), surface glycoprotein (G), and large polymerase protein (L). aMPV is currently classified into four subtypes (aMPV-A, -B, -C, and -D), but two new unclassified subtypes have recently been discovered in a Monk parakeet and a great black-backed gull.

The first detection of aMPV occurred in turkeys in South Africa in 1978 and it was later determined to be aMPV-A. aMPV-B was first detected in Europe during the 1980s. aMPV-C was first identified in turkeys in the USA in 1996. aMPV-D was retrospectively identified in archival samples from turkeys in France in 1985, but it has not been reported since.

After the first detection in Colorado, USA, in 1996, aMPV-C became endemic in multiple states of the USA during the late 1990s and early 2000s. However, after the implementation of live attenuated vaccines together with outbreak tracing and containment measures, aMPV-C has not been detected in poultry or wildlife in the USA for over a decade. aMPV-A and aMPV-B had not previously been identified in the USA, but the status changed recently. The emergence of aMPV-A in California and aMPV-B in North Carolina and Virginia in turkeys and broilers was reported for the first time in the USA in late 2023 and early 2024, followed by rapid spread of the viruses to most poultry-producing regions nationwide. According to the U.S. Animal Health Association Turkey Industry survey released in October 2024, aMPV was ranked the number one disease threat. While enhancing management practices and biosecurity measures are essential for controlling and preventing the spread of aMPV, vaccination remains a critical tool for minimizing illness and economic losses, especially by priming the immunity with a live vaccine and boosting the immunity with an inactivated vaccine. Two experimental autogenous vaccines containing inactivated aMPV-B strain of the U.S. origin, manufactured by Merck Animal Health/Cambridge Technologies and Ceva, respectively, have been approved by the USDA for use. However, modified live virus (MLV) vaccines based on aMPV-A or aMPV-B of the U.S. origin are not available yet in the USA. As of Jan. 30, 2025, USDA has approved the importation of four live aMPV vaccines and two inactivated aMPV vaccines from other countries for emergency use in the USA to control aMPV. These imported vaccines include: live vaccine Vaxxon SHS (aMPV-A, Vaxxinova Italy), live vaccine NEMOVAC (aMPV-B, Boehringer Ingelheim) for use in chickens, live vaccine AVIFFA RTI (aMPV-B, Boehringer Ingelheim) for use in turkeys, live vaccine Poulvac TRT (aMPV-A, Zoetis Spain), inactivated vaccine HIPRAVIAR TRT (aMPV-B, HIPRA), and inactivated vaccine TUR-3 (aMPV-B, Boehringer Ingelheim). Although these imported aMPV MLV vaccines are approved for the emergency use in the USA, it is essential to develop MLV vaccines using the U.S. aMPV-A and aMPV-B strains.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicates otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “adjuvant” refers to a compound that enhances the effectiveness of the vaccine and may be added to the formulation that includes the immunizing agent. Adjuvants provide enhanced immune response even after administration of only a single dose of the vaccine. Adjuvants may include, for example, aluminum hydroxide and aluminum phosphate, saponins, e.g., Quil A, QS-21 (Cambridge Biotech Inc., Cambridge Mass.), GPI-0100 (Galenica Pharmaceuticals, Inc., Birmingham, Ala.), non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or an acrylic acid polyol cross-linked polymer. The emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. In certain embodiments, the emulsifiers are nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the PLURONIC® brand products, especially L121. See Hunter et al., The Theory and Practical Application of Adjuvants (Ed. Stewart-Tull, D. E. S.) John Wiley and Sons, NY, pp 51-94 (1995) and Todd et al., Vaccine 15:564-570 (1997). In certain embodiments, the adjuvant is at a concentration of about 0.01 to about 50%, at a concentration of about 2% to 30%, at a concentration of about 5% to about 25%, at a concentration of about 7% to about 22%, or at a concentration of about 10% to about 20% by volume of the final product. Examples of suitable adjuvants are described in U.S. Patent Application Publication No. US2004/0213817 A1. “Adjuvanted” refers to a composition that incorporates or is combined with an adjuvant.

“Antibodies” refers to polyclonal and monoclonal antibodies, chimeric, and single chain antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

An “attenuated” aMPV as used herein refers to an aMPV which is capable of infecting and/or replicating in a susceptible host but is non-pathogenic or less-pathogenic to the susceptible host. For example, the attenuated virus may cause no observable/detectable clinical manifestations, or less clinical manifestations, or less severe clinical manifestations, or exhibit a reduction in virus replication efficiency and/or infectivity, as compared with the related field isolated strains. The clinical manifestations of aMPV infection can include, without limitation, sinusitis, swollen head, mortality, and reduced egg production.

The term “inactivated” and “inactivated virus” refers to a previously virulent virus that has been irradiated (UV, X-ray, or gamma radiation), heated or chemically treated to inactivate, kill, or otherwise modify the virus to substantially eliminate its virulent properties while retaining its immunogenicity. In certain embodiments, the inactivated viruses disclosed herein are inactivated by treatment with an inactivating agent. Suitable inactivating agents include beta propiolactone, binary or beta-ethyleneimine (BEI), glutaraldehyde, ozone, and Formalin (formaldehyde).

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylenediaminetetraacetic acid, among others.

An “epitope” is an antigenic determinant that is immunologically active in the sense that once administered to the host, it is able to evoke an immune response of the humoral (B cells) and/or cellular type (T cells). These are particular chemical groups or peptide sequences on a molecule that are antigenic. An antibody specifically binds a particular antigenic epitope on a polypeptide. In the subject, most antigens will present several or even many antigenic determinants simultaneously. Such a polypeptide may also be qualified as an immunogenic polypeptide and the epitope may be identified as described further.

An “immunogenic composition” refers to a composition of matter that comprises at least one antigen, which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the composition or vaccine of interest. In certain embodiments, the host will display either a therapeutic or protective immunological response such that resistance to new infection will be enhanced and/or the clinical severity of the disease reduced. Such protection will be demonstrated by either a reduction or lack of clinical signs normally displayed by an infected host, a quicker recovery time and/or a lowered duration or bacterial titer in the tissues or body fluids or excretions of the infected host compared to a healthy control. In certain embodiments, the reduction in symptoms is statistically significant when compared to a control.

The term “immunogenic fragment” as used herein refers to a polypeptide or a fragment of a polypeptide, or a nucleotide sequence encoding the same which comprises an allele-specific motif, an epitope or other sequence such that the polypeptide or the fragment will bind an MHC molecule and induce a cytotoxic T lymphocyte (“CTL”) response, and/or a B cell response (for example, antibody production), and/or T-helper lymphocyte response, and/or a delayed type hypersensitivity (DTH) response against the antigen from which the immunogenic polypeptide or the immunogenic fragment is derived. A DTH response is an immune reaction in which T cell-dependent macrophage activation and inflammation cause tissue injury. A DTH reaction to the subcutaneous injection of antigen is often used as an assay for cell-mediated immunity.

An “infectious DNA molecule”, for purposes of the present disclosure, is a DNA molecule that encodes the necessary elements for viral replication, transcription, and translation into a functional virion in a suitable host cell.

The term “isolated” is used to indicate that a cell, peptide, or nucleic acid is separated from its native environment. Isolated peptides and nucleic acids may be substantially pure, i.e. essentially free of other substances with which they may be bound in nature.

For purposes of the present disclosure, the nucleotide sequence of a second polynucleotide molecule (either RNA or DNA) is “homologous” to the nucleotide sequence of a first polynucleotide molecule, or has “identity” to said first polynucleotide molecule, where the nucleotide sequence of the second polynucleotide molecule encodes the same polypeptide as the nucleotide sequence of the first polynucleotide molecule as based on the degeneracy of the genetic code, or when it encodes a polypeptide that is sufficiently similar to the polypeptide encoded by the nucleotide sequence of the first polynucleotide molecule so as to be useful in practicing the present disclosure. Homologous polynucleotide sequence also refers to sense and anti-sense strands, and in all cases to the complement of any such strands. For purposes of the present disclosure, a polynucleotide molecule is useful in practicing the present disclosure, and is therefore homologous or has identity, where it can be used as a diagnostic probe to detect the presence of aMPV or viral polynucleotide in a fluid or tissue sample of an infected bird, e.g. by standard hybridization or amplification techniques. Generally, the nucleotide sequence of a second polynucleotide molecule is homologous to the nucleotide sequence of a first polynucleotide molecule if it has at least about 70% nucleotide sequence identity to the nucleotide sequence of the first polynucleotide molecule as based on the BLASTN algorithm (National Center for Biotechnology Information, otherwise known as NCBI, (Bethesda, Md., USA) of the United States National Institutes of Health). In a specific example for calculations according to the practice of the present disclosure, reference is made to BLASTP 2.2.6 [Tatusova TA and TL Madden, “BLAST 2 sequences—a new tool for comparing protein and nucleotide sequences.” (1999) FEMS Microbiol Lett. 174:247-250.]. Briefly, two amino acid sequences are aligned to optimize the alignment scores using a gap opening penalty of 10, a gap extension penalty of 0.1, and the “blosum62” scoring matrix of Henikoff and Henikoff (Proc. Natl. Acad. Sci. USA 89:10915-10919. 1992). The percent identity is then calculated as: Total number of identical matches X 100/divided by the length of the longer sequence+number of gaps introduced into the longer sequence to align the two sequences.

In certain embodiments, a homologous nucleotide sequence has at least 75%, at least 80%, at least 85%, at least 90%, at least 95%, at least 96%, at least 97%, at least 98%, at least 99%, and at least 99.5% nucleotide sequence identity. Since the genetic code is degenerate, a homologous nucleotide sequence can include any number of “silent” base changes, i.e. nucleotide substitutions that nonetheless encode the same amino acid.

A homologous nucleotide sequence can further contain non-silent mutations, i.e. base substitutions, deletions, or additions resulting in amino acid differences in the encoded polypeptide, so long as the sequence remains at least about 70% identical to the polypeptide encoded by the first nucleotide sequence or otherwise is useful for practicing the present disclosure. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine. Amino acids can be classified according to physical properties and contribution to secondary and tertiary protein structure. A conservative substitution is thus recognized in the art as a substitution of one amino acid for another amino acid that has similar properties, and exemplary conservative substitutions may be found in WO 97/09433, page 10, published Mar. 13, 1997 (PCT/GB96/02197, filed Sep. 6, 1996). Alternatively, conservative amino acids can be grouped as described in Lehninger, (Biochemistry, Second Edition; Worth Publishers, Inc. NY: NY (1975), pp. 71-77). Protein sequences can be aligned using both Vector NTI Advance 11.5 and CLUSTAL 2.1 multiple sequence alignment. As used herein the recitation of a particular amino acid or nucleotide sequence shall include all silent mutations with respect to nucleic acid sequence and any and all conservatively modified variants with respect to amino acid sequences.

Homologous nucleotide sequences can be determined by comparison of nucleotide sequences, for example by using BLASTN, above. Alternatively, homologous nucleotide sequences can be determined by hybridization under selected conditions. For example, the nucleotide sequence of a second polynucleotide molecule is homologous to SEQ ID NO: 1 (or any other particular polynucleotide sequence) if it hybridizes to the complement of SEQ ID NO: 1 under moderately stringent conditions, e.g., hybridization to filter-bound DNA in 0.5 M NaHPO₄, 7% sodium dodecyl sulfate (SDS), 1 mM EDTA at 65° C., and washing in 0.2×SSC/0.1% SDS at 42° C. (see Ausubel et al editors, Protocols in Molecular Biology, Wiley and Sons, 1994, pp. 6.0.3 to 6.4.10), or conditions which will otherwise result in hybridization of sequences that encode an aMPV. Modifications in hybridization conditions can be empirically determined or precisely calculated based on the length and percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N. Y. (1989), pp. 9.47 to 9.51.

In certain embodiments, a second nucleotide sequence is homologous to SEQ ID NO: 1 (or any other sequence disclosed herein) if it hybridizes to the complement of SEQ ID NO: 1 under highly stringent conditions, e.g. hybridization to filter-bound DNA in 0.5 M NaHPO₄, 7% SDS, 1 mM EDTA at 65° C., and washing in 0.1×SSC/0.1% SDS at 68° C., as is known in the art.

It is furthermore to be understood that the isolated polynucleotide molecules and the isolated RNA molecules of the present disclosure include both synthetic molecules and molecules obtained through recombinant techniques, such as by in vitro cloning and transcription.

As used herein, “a pharmaceutically acceptable carrier” or “pharmaceutical carrier” includes any and all excipients, solvents, growth media, dispersion media, coatings, adjuvants, stabilizing agents, diluents, preservatives, inactivating agents, antimicrobial, antibacterial and antifungal agents, isotonic agents, adsorption delaying agents, and the like. Such ingredients include those that are safe and appropriate for use in veterinary applications. Pharmaceutically acceptable carriers are typically non-toxic, inert, solid or liquid carriers.

As used herein, “subject” refers to avian and non-avian animals. An “avian subject” may be any member of the class Aves including, but not limited to, chickens, turkeys, ducks, geese, or other fowl. The term “poultry” refers generally to any avian subject that is agriculturally relevant, e.g., chickens, ducks, geese, guinea fowl, turkeys, quail, pheasants, and the like. The term “subject” does not denote a particular age or sex. In certain embodiments, the subject is a chicken or a turkey. In certain embodiments, the subject is at risk of being infected by aMPV.

A “susceptible” host as used herein refers to a cell or an animal that can be infected by aMPV. When introduced to a susceptible animal, an attenuated aMPV may also induce an immunological response against the aMPV or its antigen, and thereby render the animal immunity against aMPV infection.

The term “vaccine” refers to an antigenic preparation used to produce immunity to a disease, in order to prevent or ameliorate the effects of infection. Vaccines are typically prepared using a combination of an immunologically effective amount of an immunogen together with an adjuvant effective for enhancing the immune response of the vaccinated subject against the immunogen.

Vaccine formulations will contain a “therapeutically effective amount” of the active ingredient, that is, an amount capable of eliciting an induction of an immunoprotective response in a subject to which the composition is administered. In the treatment and prevention of aMPV, for example, a “therapeutically effective amount” would be an amount that enhances resistance of the vaccinated subject to new infection and/or reduces the clinical severity of the disease. Such protection will be demonstrated by either a reduction or lack of symptoms normally displayed by a subject infected with aMPV, a quicker recovery time and/or a lowered count of virus particles. Vaccines can be administered prior to infection, as a preventative measure against aMPV. Alternatively, vaccines can be administered after the subject already has contracted a disease. Vaccines given after exposure to aMPV may be able to attenuate the disease, triggering a superior immune response than the natural infection itself.

The present disclosure provides for reduction of the incidence of and/or severity of clinical symptoms associated with aMPV infection. In certain embodiments, the severity and/or incidence of clinical symptoms in subjects receiving the immunogenic composition of the present disclosure are reduced at least 10% in comparison to subjects not receiving such an administration when both groups (subjects receiving and subjects not receiving the composition) are challenged with or exposed to infection by aMPV. In certain embodiments, the incidence or severity is reduced at least 20%, at least 30%, at least 40%, at least 50%, at least 60%, at least 70%, at least 80%, at least 90%, at least 95%, or at least 100%, wherein the subjects receiving the composition of the present disclosure exhibit no clinical symptoms, or alternatively exhibit clinical symptoms of reduced severity.

For the purpose of the practice of all aspects of the disclosure, it is well known to those skilled in the art that there is no absolute immunological boundary in immunological assays in regard to subjects that are seronegative for exposure to a particular antigen or pathogen, and those that are seropositive (having been exposed to a vaccine or pathogen). Nonetheless, those skilled in the art would recognize that in serum neutralization assays, seropositive subjects would generally be detected at least up to a 1:1000 serum dilution, whereas a seronegative subject would be expected not to neutralize at a higher dilution than about 1:20 or 1:10.

### Vaccine and Immunogenic Compositions

The disclosure also relates to an immunogenic composition, suitable to be used as a vaccine, which comprises an aMPV strain according to the disclosure. In certain embodiments, the immunogenic compositions according to the disclosure elicit a specific humoral immune response toward the aMPV comprising neutralizing antibodies.

The immunogenic compositions based upon the strains disclosed herein can provide live attenuated viruses which exhibit high immunogenicity while at the same time not producing dangerous pathogenic or lethal effects.

The immunogenic and vaccine compositions of this disclosure are not, however, restricted to any particular type or method of preparation. These include, but are not limited to, infectious DNA vaccines (i.e., using plasmids, vectors or other conventional carriers to directly inject DNA into avian subjects), live vaccines, modified live vaccines, inactivated vaccines, subunit vaccines, attenuated vaccines, genetically engineered vaccines, etc. These vaccines are prepared by standard methods known in the art.

In certain embodiments, the immunogenic compositions comprise a live attenuated aMPV and a pharmaceutically acceptable carrier. The pharmaceutically acceptable carrier can be, e.g., water, a stabilizer, a preservative, culture medium, or a buffer. Immunogenic and vaccine compositions comprising the attenuated aMPV of the disclosure can be prepared in the form of a suspension or in a lyophilized form or, alternatively, in a frozen form. If frozen, glycerol or other similar agents may be added to enhance stability when frozen. The advantages of live attenuated viruses, in general, include the presentation of all the relevant immunogenic determinants of an infectious agent in its natural form to the host's immune system, and the need for relatively small amounts of the immunizing agent due to the ability of the agent to multiply in the vaccinated host.

Attenuation of the virus for a live vaccine, so that it is insufficiently pathogenic to substantially harm the vaccinated target subject, may be accomplished by known procedures, including by serial passaging. The following references provide various general methods for attenuation, and are suitable for attenuation or further attenuation of any of the strains of the present disclosure: B. Neuman et al., Journal of Virology, vol. 79, No. 15, pp. 9665-9676, 2005; J. Netland et al., Virology, v 399 (1), pp. 120-128, 2010; Y-P Huang et al., “Sequence changes of infectious bronchitis virus isolates in the 3′ 7.3 kb of the genome after attenuating passage in embryonated eggs, Avian Pathology, v. 36 (1), (Abstract), 2007; and S. Hingley et al., Virology, v. 200 (1) 1994, pp. 1-10; see U.S. Pat. No. 3,914,408; and Ortego et al., Virology, vol. 308 (1), pp. 13-22, 2003. In certain embodiments, the live attenuated aMPV is attenuated by passaging in cell culture such that when the attenuated virus is administered to an avian subject it fails to cause clinical signs of aMPV but is capable of inducing an immune response that immunizes the avian subject against pathogenic forms of aMPV. In certain embodiments, the aMPV is passaged in Vero cells.

In certain embodiments, the aMPV comprises a nucleotide substitution at one or more of the following positions as determined by reference to SEQ ID NO: 1 or 5: position 959 (encoding the nucleoprotein), position 1398 (encoding the phosphoprotein), position 2830 (encoding the matrix protein), position 3902 (encoding the fusion protein), position 3903 (encoding the fusion protein), position 5939 (in an intergenic region), position 5964 (in an intergenic region), position 8579 (encoding the large polymerase), position 10958 (encoding the large polymerase), or position 11118 (encoding the large polymerase). In certain embodiments, the aMPV comprises one or more of the following nucleotide substitutions: a G at position 959, a T at position 1398, a T at position 2830, an A at position 3902, a G at position 3903, an A at position 5939, an A at position 5964, a C at position 8579, a T at position 10958, or a G at position 11118 as determined by reference to SEQ ID NO: 1 or 5.

In certain embodiments, the aMPV comprises a nucleotide substitution at one or more of the following positions as determined by reference to SEQ ID NO: 9: position 3262 (encoding the fusion protein) or position 4110 (encoding the fusion protein). In certain embodiments, the aMPV comprises one or more of the following nucleotide substitutions: a C at position 3262 or a T at position 4110 as determined by reference to SEQ ID NO: 9. In certain embodiments, the aMPV comprises a nucleotide substitution at one or more of the following positions as determined by reference to SEQ ID NO: 13: position 5212 (encoding the M2.2 protein), position 5295 (encoding the M2.2 protein), or position 7041 (encoding the surface glycoprotein). In certain embodiments, the aMPV comprises one or more of the following nucleotide substitutions: a G at position 5212, an A at position 5295, or a T at position 7041 as determined by reference to SEQ ID NO: 13.

In certain embodiments, the aMPV encodes a phosphoprotein with an amino acid substitution at the following position as determined by reference to SEQ ID NO: 17: position 53. In certain embodiments, the aMPV encodes a phosphoprotein with the following amino acid substitution: a serine at position 53 as determined by reference to SEQ ID NO: 17. In certain embodiments, the phosphoprotein has at least 90%, 95%, 96%, 97%, 98%, 99%, or 99.5% sequence identity to the amino acid sequence set forth in SEQ ID NO: 17.

In certain embodiments, the aMPV encodes a matrix protein with an amino acid substitution at the following position as determined by reference to SEQ ID NO: 18: position 243. In certain embodiments, the aMPV encodes a matrix protein with the following amino acid substitution: a tyrosine at position 243 as determined by reference to SEQ ID NO: 18. In certain embodiments, the matrix protein has at least 90%, 95%, 96%, 97%, 98%, 99%, or 99.5% sequence identity to the amino acid sequence set forth in SEQ ID NO: 18.

In certain embodiments, the aMPV encodes a fusion protein with an amino acid substitution at one or more of the following positions as determined by reference to SEQ ID NO: 19: position 112, position 323, or position 395. In certain embodiments, the aMPV encodes a fusion protein with one or more of the following amino acid substitutions: an alanine at position 112, a glycine or a lysine at position 323, or a tyrosine at position 395 as determined by reference to SEQ ID NO: 19. In certain embodiments, the fusion protein has at least 90%, 95%, 96%, 97%, 98%, 99%, or 99.5% sequence identity to the amino acid sequence set forth in SEQ ID NO: 19.

In certain embodiments, the aMPV encodes a M2.2 protein with an amino acid substitution at one or more of the following positions as determined by reference to SEQ ID NO: 20: position 41 or position 69. In certain embodiments, the aMPV encodes a M2.2 protein with one or more of the following amino acid substitutions: a glycine at position 41 or an asparagine at position 69 as determined by reference to SEQ ID NO: 20. In certain embodiments, the M2.2 protein has at least 90%, 95%, 96%, 97%, 98%, 99%, or 99.5% sequence identity to the amino acid sequence set forth in SEQ ID NO: 20.

In certain embodiments, the aMPV encodes a surface glycoprotein with an amino acid substitution at the following position as determined by reference to SEQ ID NO: 21: position 348. In certain embodiments, the aMPV encodes a surface glycoprotein with the following amino acid substitution: a leucine at position 348 as determined by reference to SEQ ID NO: 21. In certain embodiments, the surface glycoprotein has at least 90%, 95%, 96%, 97%, 98%, 99%, or 99.5% sequence identity to the amino acid sequence set forth in SEQ ID NO: 21.

In certain embodiments, the aMPV encodes a large polymerase with an amino acid substitution at one or more of the following positions as determined by reference to SEQ ID NO: 22: position 448 or position 1294. In certain embodiments, the aMPV encodes a large polymerase with one or more of the following amino acid substitutions: a leucine at position 448 or an arginine at position 1294 as determined by reference to SEQ ID NO: 22. In certain embodiments, the large polymerase has at least 90%, 95%, 96%, 97%, 98%, 99%, or 99.5% sequence identity to the amino acid sequence set forth in SEQ ID NO: 22.

Additional genetically engineered vaccines, which are desirable in the present disclosure, are produced by techniques known in the art. Such techniques involve, but are not limited to, further manipulation of recombinant DNA, modification of or substitutions to the amino acid sequences of the recombinant proteins and the like.

Genetically engineered vaccines based on recombinant DNA technology are made, for instance, by identifying alternative portions of the viral gene encoding proteins responsible for inducing a stronger immune or protective response in avian subjects (e.g., nucleoprotein (N), phosphoprotein (P), matrix protein (M), fusion protein (F), second matrix proteins (M2.1 and M2.2), small hydrophobic protein (SH), surface glycoprotein (G), large polymerase protein (L)).

Various subtypes or isolates of the viral protein genes can be subjected to the DNA-shuffling method. The resulting heterogeneous chimeric viral proteins can be used broad protecting subunit vaccines. Alternatively, such chimeric viral genes or immuno-dominant fragments can be cloned into standard protein expression vectors, such as the baculovirus vector, and used to infect appropriate host cells (see, for example, O'Reilly et al., “Baculovirus Expression Vectors: A Lab Manual,” Freeman & Co., 1992). The host cells are cultured, thus expressing the desired vaccine proteins, which can be purified to the desired extent and formulated into a suitable vaccine product.

If the clones retain any undesirable natural abilities of causing disease, it is also possible to pinpoint the nucleotide sequences in the viral genome responsible for any residual virulence, and genetically engineer the virus avirulent through, for example, site-directed mutagenesis. Site-directed mutagenesis is able to add, delete or change one or more nucleotides (see, for instance, Zoller et al., DNA 3:479-488, 1984). An oligonucleotide is synthesized containing the desired mutation and annealed to a portion of single stranded viral DNA. The hybrid molecule, which results from that procedure, is employed to transform bacteria. Then double-stranded DNA, which is isolated containing the appropriate mutation, is used to produce full-length DNA by ligation to a restriction fragment of the latter that is subsequently transfected into a suitable cell culture. Ligation of the genome into the suitable vector for transfer may be accomplished through any standard technique known to those of ordinary skill in the art. Transfection of the vector into host cells for the production of viral progeny may be done using any of the conventional methods such as calcium-phosphate or DEAE-dextran mediated transfection, electroporation, protoplast fusion and other well-known techniques (e.g., Sambrook et al., “Molecular Cloning: A Laboratory Manual,” Cold Spring Harbor Laboratory Press, 1989). The cloned virus then exhibits the desired mutation. Alternatively, two oligonucleotides can be synthesized which contain the appropriate mutation. These may be annealed to form double-stranded DNA that can be inserted in the viral DNA to produce full-length DNA.

An immunologically effective amount of the vaccines of the present disclosure is administered to an avian subject in need of protection against viral infection. The immunologically effective amount or the immunogenic amount that inoculates the avian subject can be easily determined or readily titrated by routine testing. An effective amount is one in which a sufficient immunological response to the vaccine is attained to protect the avian subject exposed to the aMPV virus. In certain embodiments, the avian subject is protected to an extent in which one to all of the adverse physiological symptoms or effects of the viral disease are significantly reduced, ameliorated or totally prevented.

The vaccine or immunogenic compositions of the present disclosure can be formulated following accepted convention to include acceptable carriers for subjects, such as standard buffers, stabilizers, diluents, preservatives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Other suitable vaccine vehicles and additives, including those that are particularly useful in formulating modified live vaccines, are known or will be apparent to those skilled in the art. See, e.g., Remington's Pharmaceutical Science, 18th ed., 1990, Mack Publishing, which is incorporated herein by reference.

The vaccine or immunogenic compositions of the present disclosure may further comprise one or more additional immunomodulatory components such as, e.g., an adjuvant or cytokine, among others. Non-limiting examples of adjuvants that can be used in the vaccine of the present disclosure include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN® adjuvant, saponin, Quil A or other saponin fraction, monophosphoryl lipid A, ionic polysaccharides, and Avridine lipid-amine adjuvant. Non-limiting examples of oil-in-water emulsions useful in the vaccine of the disclosure include modified SEAM62 and SEAM 1/2 formulations. Modified SEAM62 is an oil-in-water emulsion containing 5% (v/v) squalene (Sigma), 1% (v/v) SPAN® 85 detergent (ICI Surfactants), 0.7% (v/v) TWEEN® 80 detergent (ICI Surfactants), 2.5% (v/v) ethanol, 200 μg/ml Quil A, 100 μg/ml cholesterol, and 0.5% (v/v) lecithin. Modified SEAM 1/2 is an oil-in-water emulsion comprising 5% (v/v) squalene, 1% (v/v) SPAN® 85 detergent, 0.7% (v/v) TWEEN® 80 detergent, 2.5% (v/v) ethanol, 100 μg/ml Quil A, and 50 μg/ml cholesterol. Other immunomodulatory agents that can be included in the vaccine include, e.g., one or more interleukins, interferons, or other known cytokines. Additional adjuvant systems permit for the combination of both T-helper and B-cell epitopes, resulting in one or more types of covalent T-B epitope linked structures, which may be additionally lipidated, such as those described in WO2006/084319, WO2004/014957, and WO2004/014956. The vaccine compositions of the disclosure may or may not include adjuvants.

The immunogenic and vaccine compositions of the disclosure can further comprise pharmaceutically acceptable carriers, excipients and/or stabilizers (see e.g. Remington: The Science and practice of Pharmacy, 2005, Lippincott Williams), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as Mercury ((o-carboxyphenyl)thio) ethyl sodium salt, octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), TWEEN® or PLURONICS®.

Vaccines of the present disclosure can optionally be formulated for sustained release of the virus, infectious DNA molecule, plasmid, or viral vector of the present disclosure. Examples of such sustained release formulations include virus, infectious DNA molecule, plasmid, or viral vector in combination with composites of biocompatible polymers, such as, e.g., poly (lactic acid), poly(lactic-co-glycolic acid), methylcellulose, hyaluronic acid, collagen and the like. The structure, selection and use of degradable polymers in drug delivery vehicles have been reviewed in several publications, including A. Domb et al., 1992, Polymers for Advanced Technologies 3:279-292, which is incorporated herein by reference. Additional guidance in selecting and using polymers in pharmaceutical formulations can be found in texts known in the art, for example M. Chasin and R. Langer (eds), 1990, “Biodegradable Polymers as Drug Delivery Systems” in: Drugs and the Pharmaceutical Sciences, Vol. 45, M. Dekker, NY, which is also incorporated herein by reference. Alternatively, or additionally, the virus, plasmid, or viral vector can be microencapsulated to improve administration and efficacy. Methods for microencapsulating antigens are well-known in the art, and include techniques described, e.g., in U.S. Pat. Nos. 3,137,631; 3,959,457; 4,205,060; 4,606,940; 4,744,933; 5,132,117; and International Patent Publication WO 95/28227, all of which are incorporated herein by reference.

Liposomes can also be used to provide for the sustained release of virus, plasmid, viral protein, or viral vector. Details concerning how to make and use liposomal formulations can be found in, among other places, U.S. Pat. Nos. 4,016,100; 4,452,747; 4,921,706; 4,927,637; 4,944,948; 5,008,050; and 5,009,956, all of which are incorporated herein by reference.

An effective amount of any of the above-described vaccines can be determined by conventional means, starting with a low dose of virus, viral protein plasmid or viral vector, and then increasing the dosage while monitoring the effects. An effective amount may be obtained after a single administration of a vaccine or after multiple administrations of a vaccine. Known factors can be taken into consideration when determining an optimal dose per subject. These include the species, size, age and general condition of the subject, the presence of other drugs in the subject, and the like. In certain embodiments, the actual dosage is chosen after consideration of the results from other animal studies.

One method of detecting whether an adequate immune response has been achieved is to determine seroconversion and antibody titer in the subject after vaccination. The timing of vaccination and the number of boosters, if any, may be determined by a doctor or veterinarian based on analysis of all relevant factors, some of which are described above.

The effective dose amount of virus, protein, infectious nucleotide molecule, plasmid, or viral vector, of the present disclosure can be determined using known techniques, taking into account factors that can be determined by one of ordinary skill in the art such as the weight of the subject to be vaccinated. In certain embodiments, the dose amount of virus of the present disclosure in a vaccine of the present disclosure ranges from about 10¹ to about 10⁹ pfu (plaque forming units), from about 10² to about 10⁸ pfu, or from about 10³ to about 10⁷ pfu. In certain embodiments, the dose amount of a plasmid of the present disclosure in a vaccine of the present disclosure ranges from about 0.1 μg to about 100 mg, from about 1 μg to about 10 mg, or from about 10 μg to about 1 mg. In certain embodiments, the dose amount of an infectious DNA molecule of the present disclosure in a vaccine of the present disclosure ranges from about 0.1 μg to about 100 mg, from about 1 μg to about 10 mg, or from about 10 μg to about 1 mg. In certain embodiments, the dose amount of a viral vector of the present disclosure in a vaccine of the present disclosure ranges from about 10¹ pfu to about 10⁹ pfu, from about 10² pfu to about 10⁸ pfu, or from about 10³ to about 10⁷ pfu. In certain embodiments, a suitable dosage size ranges from about 0.5 ml to about 10 ml, or from about 1 ml to about 5 ml. Suitable doses for viral protein or peptide vaccines according to the practice of the present disclosure range generally from 1 to 50 micrograms per dose, or higher amounts as may be determined by standard methods, with the amount of adjuvant to be determined by recognized methods in regard of each such substance.

When provided therapeutically, the vaccine is provided in an effective amount upon the detection of a sign of actual infection. Suitable dose amounts for treatment of an existing infection include between about 10¹ and about 10⁶ log 10 TCID₅₀, or higher, of virus per dose (minimum immunizing dose to vaccine release). “TCID₅₀” refers to “tissue culture infective dose” and is defined as that dilution of a virus required to infect 50% of a given batch of inoculated cell cultures. Various methods may be used to calculate TCID₅₀, including the Spearman-Karber method. For a description of the Spearman-Karber method, see B. W. Mahy & H. O. Kangro, Virology Methods Manual, p. 25-46 (1996). A composition is said to be “pharmacologically acceptable” if its administration can be tolerated by a recipient. Such a composition is said to be administered in a “therapeutically or prophylactically effective amount” if the amount administered is physiologically significant.

At least one vaccine or immunogenic composition of the present disclosure can be administered by any means that achieve the intended purpose, using a composition as described herein. For example, route of administration of such a composition can be by parenteral, oral, oronasal, intranasal, intratracheal, topical, subcutaneous, intramuscular, transcutaneous, intradermal, intraperitoneal, intraocular, and intravenous administration. In certain embodiments, the composition is administered in ovo. In certain embodiments, the composition is administered intramuscularly. Parenteral administration can be by bolus injection or by gradual perfusion over time. Any suitable device may be used to administer the compositions, including syringes, droppers, needleless injection devices, patches, and the like. The route and device selected for use will depend on the composition of the adjuvant, the antigen, and the subject, and such are well known to the skilled artisan. Oral administration may be direct, via water, or via feed (solid or liquid feed). When provided in liquid form, the vaccine may be lyophilized with reconstitution, or provided as a paste, for direct addition to feed (mix in or top dress) or otherwise added to water or liquid feed. In certain embodiments, the compositions are administered to greater than one subject at a time through means known in the art, for example, through mass intranasal administration of a group of animals. In certain embodiments, the compositions of the current disclosure are administered by aerosol delivery to a flock of birds, for example, chickens or turkeys.

The appropriate dose of the immunogenic composition of the present disclosure depends on several variables such as the formulation, the route of administration, the subject's age, the subject's weight, the time of administration, the excretion rate, and reaction irritability. One of ordinary skill in the art can determine the appropriate dose by administering the antigen to the subject and assaying for an increase or, if applicable, a decrease in the immune response.

The immunogenic compositions may comprise proteins and/or antigens from at least one additional pathogen (“non-aMPV”). The additional pathogen may be any pathogen that causes illness and/or an infection in birds. Exemplary pathogens include, but are not limited to, Pasteurella multocida, Mycoplasma gallisepticum, Salmonella enterica, Escherichia coli, Clostridium tetani, Clostridium perfringens, Eimeria spp., Avian influenza, Marek's disease virus, Infectious bronchitis virus, Fowlpox virus, Newcastle disease virus, Avian encephalomyelitis virus, or a combination thereof.

Embodiments herein relating to “vaccine compositions” of the disclosure are also applicable to embodiments relating to “immunogenic compositions” of the disclosure, and vice versa.

### Polynucleotides and Polypeptides

Representative embodiments of the disclosure include an isolated polynucleotide sequence that comprises a polynucleotide selected from: (a) SEQ ID NO: 1, 2, 3, 4, 5, 6, 7, 8, 9, 10, 11, 12, 13, 14, 15, or 16; (b) the complement of any sequence in (a); (c) a polynucleotide that hybridizes with a sequence of (a) or (b) under stringent conditions defined as hybridizing to filter bound DNA in 0.5M NaHPO₄, 7% SDS, 1 mM EDTA at 65° C., and washing in 0.1×SSC/0.1% SDS at 68° C.; (d) a polynucleotide that is at least 70% identical to the polynucleotide of (a) or (b); (e) a polynucleotide that is at least 80% identical to the polynucleotide of (a) or (b); (f) a polynucleotide that is at least 90% identical to the polynucleotide of (a) or (b); (g) a polynucleotide that is at least 95% identical to the polynucleotide of (a) or (b); (h) a polynucleotide that is at least 98% identical to the polynucleotide of (a) or (b); and (i) a polynucleotide that is at least 99% identical to the polynucleotide of (a) or (b).

The disclosure also provides a polypeptide encoded by any of the open reading frames of SEQ ID NO: 1, 2, 3, 4, 5, 6, 7, 8, 9, 10, 11, 12, 13, 14, 15, or 16, combinations thereof, or a polypeptide that is at least 70%, at least 80%, at least 90%, at least 95%, at least 98%, or at least 99% identical thereto, domains thereof, or to a fragment thereof, including the option that additional otherwise identical amino acids are replaced by conservative substitutions.

The disclosure also provides a polypeptide encoded by any of the open reading frames of the aMPV strains of the disclosure, or a polypeptide that is at least 70%, at least 80%, at least 90%, at least 95%, at least 98%, or at least 99% identical thereto, or to a fragment thereof, including the option that additional otherwise identical amino acids are replaced by conservative substitutions.

The polynucleotide and amino acid sequence information provided by the present disclosure also makes possible the systematic analysis of the structure and function of the viral genes and their encoded gene products. Knowledge of a polynucleotide encoding a viral gene product of the disclosure also makes available anti-sense polynucleotides which recognize and hybridize to polynucleotides encoding a polypeptide of the disclosure, or a fragment thereof. Full length and fragment anti-sense polynucleotides are useful in this respect. The worker of ordinary skill will appreciate that fragment anti-sense molecules of the disclosure include (i) those which specifically recognize and hybridize to a specific RNA (as determined by sequence comparison of DNA encoding a viral polypeptide of the disclosure) as well as (ii) those which recognize and hybridize to RNA encoding variants of the encoded proteins. Antisense polynucleotides that hybridize to RNA/DNA encoding other aMPV peptides are also identifiable through sequence comparison to identify characteristic, or signature sequences for the family of molecules, further of use in the study of antigenic domains in aMPV polypeptides, and may also be used to distinguish between infection of a host animal with remotely related non-aMPV members of the Pneumoviridae family.

### Antibodies

Also contemplated by the present disclosure are anti-aMPV antibodies (e.g., monoclonal and polyclonal antibodies, single chain antibodies, chimeric antibodies, and CDR-grafted antibodies, including compounds which include CDR sequences which specifically recognize an aMPV polypeptide of the disclosure). The term “specific for” indicates that the variable regions of the antibodies of the disclosure recognize and bind an aMPV polypeptide exclusively (i.e., are able to distinguish a single aMPV polypeptide from related polypeptides despite sequence identity, homology, or similarity found in the family of polypeptides), and which are permitted (optionally) to interact with other proteins (for example, S. aureus protein A or other antibodies in ELISA techniques) through interactions with sequences outside the variable region of the antibodies, and in particular, in the constant region of the Ab molecule. Screening assays to determine binding specificity of an antibody of the disclosure are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the aMPV polypeptides of the disclosure are also contemplated, provided that the antibodies are first and foremost specific for, as defined above, an aMPV polypeptide of the disclosure from which the fragment was derived.

For the purposes of clarity, “antibody” refers to an immunoglobulin molecule that can bind to a specific antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions. Antibodies can exist in a variety of forms including, for example, Fv, Fab′, F(ab′)₂, as well as in single chains, and include synthetic polypeptides that contain all or part of one or more antibody single chain polypeptide sequences.

### Diagnostic Kits

The present disclosure also provides diagnostic kits. The kit can be valuable for differentiating between avian subjects naturally infected with a field strain of an aMPV and avian subjects administered with any of the aMPV vaccine or immunogenic compositions described herein. The kits can also be of value because avian subjects potentially infected with field strains of aMPV virus can be detected prior to the existence of clinical symptoms and removed from the flock, or kept in isolation away from naive or vaccinated avian subjects. The kits include reagents for analyzing a sample from an avian subject for the presence of antibodies to a particular component of a specified aMPV virus. Diagnostic kits of the present disclosure can include as a component a peptide or peptides from the aMPV strains of the disclosure which is present in a field strain but not in the vaccine or immunogenic composition of interest, or vice versa, and selection of such suitable peptide domains is made possible by the extensive amino acid sequencing. As is known in the art, kits of the present disclosure can alternatively include as a component a peptide which is provided via a fusion protein. The term “fusion peptide” or “fusion protein” for purposes of the present disclosure means a single polypeptide chain consisting of at least a portion of an aMPV protein and a heterologous peptide or protein.

### EMBODIMENTS

The following numbered embodiments also form part of the present disclosure:

1. An immunogenic composition comprising an inactivated or live attenuated avian metapneumovirus (aMPV), wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16; and a pharmaceutically acceptable carrier.

2. The immunogenic composition of embodiment 1, wherein the aMPV is inactivated, optionally wherein the composition comprises at least one aMPV subtype A isolate and at least one aMPV subtype B isolate.

3. The immunogenic composition of embodiment 1, wherein the aMPV is a live attenuated virus.

4. The immunogenic composition of embodiment 3, wherein the aMPV is attenuated by passaging in cell culture such that when the attenuated virus is administered to an avian subject it fails to cause clinical signs of aMPV but is capable of inducing an immune response that immunizes the avian subject against pathogenic forms of aMPV.

5. The immunogenic composition of any one of embodiments 1-4, wherein the pharmaceutically acceptable carrier is a diluent, adjuvant, antimicrobial agent, preservative, inactivating agent, or a combination thereof.

6. The immunogenic composition of any one of embodiments 1-5, wherein the aMPV is not wild type.

7. The immunogenic composition of any one of embodiments 1-6, further comprising one or more non-aMPV inactivated or attenuated pathogens or antigenic material thereof.

8. A method for inducing an immune response against avian metapneumovirus (aMPV) in an avian subject comprising: administering to the avian subject an immunogenic composition comprising an inactivated or live attenuated aMPV, wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

9. The method of embodiment 8, wherein the aMPV is inactivated, optionally wherein the composition comprises at least one aMPV subtype A isolate and at least one aMPV subtype B isolate.

10. The method of embodiment 8, wherein the aMPV is a live attenuated virus.

11. The method of embodiment 10, wherein the aMPV is attenuated by passaging in cell culture such that when the attenuated aMPV is administered to an avian subject it fails to cause clinical signs of aMPV but is capable of inducing an immune response that immunizes the avian subject against pathogenic forms of aMPV.

12. The method of any one of embodiments 8-11, wherein the immunogenic composition further comprises a pharmaceutically acceptable carrier.

13. The method of embodiment 12, wherein the pharmaceutically acceptable carrier comprises a diluent, adjuvant, antimicrobial agent, preservative, inactivating agent, or a combination thereof.

14. The method of any one of embodiments 8-13, wherein the immunogenic composition further comprises one or more non-aMPV inactivated or attenuated pathogens or antigenic material thereof.

15. The method of any one of embodiments 8-14, wherein the avian subject is a chicken or a turkey.

16. An isolated avian metapneumovirus (aMPV) comprising a polynucleotide having at least 90%, 95%, 96%, 97%, 98%, 99%, or 99.5% sequence identity to SEQ ID NO: 1, 2, 3, 4, 5, 6, 7, 8, 9, 10, 11, 12, 13, 14, 15, or 16, and wherein the aMPV is not wild type.

17. A DNA polynucleotide encoding an avian metapneumovirus (aMPV), or an immunogenic fragment thereof, wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

18. A vector comprising the DNA polynucleotide of embodiment 17.

19. A host cell comprising the DNA polynucleotide of embodiment 17.

20. A culture of cells comprising an avian metapneumovirus (aMPV), wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

21. The culture of cells of embodiment 20, wherein the cells are Vero cells, chicken embryo fibroblast cells, or chicken embryo lung cells.

22. The culture of cells of embodiment 20 or embodiment 21, wherein the aMPV is USA/IA55601-6/2024, USA/IA56509-5/2024, USA/NC20487-GA/2024, or USA/NC23734-GA/2024, wherein a representative culture has been deposited under ATCC Accession No. ______.

23. A method of preparing a live attenuated avian metapneumovirus (aMPV) comprising: passaging an aMPV in cell culture, wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

24. The method of embodiment 23, wherein the virus is passaged in Vero cells, chicken embryo fibroblast cells, or chicken embryo lung cells.

25. The method of embodiment 23 or embodiment 24, wherein the aMPV is USA/IA55601-6/2024, USA/IA56509-5/2024, USA/NC20487-GA/2024, or USA/NC23734-GA/2024 wherein a representative culture has been deposited under ATCC Accession No. ______.

26. A vaccine composition comprising an inactivated or live attenuated avian metapneumovirus (aMPV), wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16; and a pharmaceutically acceptable carrier.

27. The vaccine composition of embodiment 26, wherein the aMPV is inactivated, optionally wherein the composition comprises at least one aMPV subtype A isolate and at least one aMPV subtype B isolate.

28. The vaccine composition of embodiment 26, wherein the aMPV is a live attenuated virus.

29. The vaccine composition of embodiment 28, wherein the aMPV is attenuated by passaging in cell culture such that when the attenuated virus is administered to an avian subject it fails to cause clinical signs of aMPV but is capable of inducing an immune response that immunizes the avian subject against pathogenic forms of aMPV.

30. The vaccine composition of any one of embodiments 26-29, wherein the pharmaceutically acceptable carrier comprises a diluent, adjuvant, antimicrobial agent, preservative, inactivating agent, or a combination thereof

31. The vaccine composition of any one of embodiments 26-30, wherein the composition comprises an adjuvant.

32. The vaccine composition of any one of embodiments 26-31, further comprising one or more non-aMPV inactivated or attenuated pathogens or antigenic material thereof.

33. The vaccine composition of any one of embodiments 26-32, wherein the vaccine is effective in a single dose program or in a two-dose program.

34. A method of treating or preventing disease caused by avian metapneumovirus (aMPV) comprising: administering to an avian subject in need thereof a vaccine composition comprising an inactivated or live attenuated aMPV, wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

35. The method of embodiment 34, wherein the aMPV is inactivated, optionally wherein the composition comprises at least one aMPV subtype A isolate and at least one aMPV subtype B isolate.

36. The method of embodiment 34, wherein the aMPV is a live attenuated virus.

37. The method of embodiment 36, wherein the aMPV is attenuated by passaging in cell culture such that when the attenuated aMPV is administered to an avian subject it fails to cause clinical signs of aMPV but is capable of inducing an immune response that immunizes the avian subject against pathogenic forms of aMPV.

38. The method of any one of embodiments 34-37, wherein the vaccine composition further comprises a pharmaceutically acceptable carrier.

39. The method of any one of embodiments 34-38, wherein the pharmaceutically acceptable carrier is a diluent, adjuvant, antimicrobial agent, preservative, inactivating agent, or a combination thereof.

40. The method of any one of embodiments 34-39, wherein the composition comprises an adjuvant.

41. The method of any one of embodiments 34-40, wherein the vaccine composition further comprises one or more non-aMPV inactivated or attenuated pathogens or antigenic material thereof.

42. The method of any one of embodiments 34-41, wherein the avian subject is a chicken or a turkey.

43. A method for determining if an avian population is in need of vaccination against avian metapneumovirus (aMPV) infection: collecting a biological sample from one or more subjects of the avian population; and detecting the presence of the aMPV in the biological sample, wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

44. The method of embodiment 43, wherein the detecting is by polymerase chain reaction, immunohistochemistry, immunofluorescence, or an indirect fluorescent antibody assay.

45. The method of embodiment 43 or embodiment 44, further comprising: administering an effective amount of the vaccine composition of any one of embodiments 26-33 to the population if the aMPV is detected.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended embodiments.

The following examples are offered by way of illustration and not by way of limitation.

### EXAMPLES

### Example 1: Isolation and Characterization of Avian Metapneumovirus Subtypes A and B Associated with the 2023-2024 Disease Outbreaks Among Poultry in the United States

Avian metapneumovirus (aMPV) subtypes A and B were first detected in U.S. poultry in late 2023 and early 2024, rapidly spreading nationwide and posing a significant threat to the industry. This study analyzed aMPV PCR data from 2,204 samples (1,158 turkey, 936 chicken, and 110 other breeds) submitted to a U.S. veterinary diagnostic laboratory between January and November 2024. A higher percentage (51.04%) of turkey samples tested PCR-positive for aMPV-A and/or aMPV-B compared to chicken samples (15.6%), with aMPV-A showing an overall higher positive rate than aMPV-B, although the positive rates varied by state. Additionally, in this study, four aMPV-A and three aMPV-B isolates were successfully obtained from clinical samples using primary chicken embryo lung and/or fibroblast cells. Two aMPV-A isolates (USA/IA55601-6/2024 and USA/IA56509-5/2024) and two aMPV-B isolates (USA/NC20487-GA/2024 and USA/NC23734-GA/2024) were adapted to grow efficiently in a Vero cell line, reaching titers of ~10⁴-10⁶ TCID₅₀/ml between passages 4 and 10 for aMPV-A and between passages 1 and 10 for aMPV-B. The whole genome sequences of the two aMPV-A and two aMPV-B isolates at different cell culture passages were determined, and the viruses were genetically stable within 10 passages in cell culture. Comparisons with 46 aMPV-A, -B, -C, and -D sequences from GenBank showed that U.S. aMPV-A and aMPV-B strains were genetically closely related within their subtypes. These cell culture-adapted U.S. aMPV-A and aMPV-B isolates provide valuable tools for further characterization of aMPV and vaccine development.

### Materials and Methods

Clinical Samples Submitted to Iowa State University Veterinary Diagnostic Laboratory (ISU VDL) for aMPV PCR Testing Between January and November 2024

A total of 2,204 clinical samples tested by aMPV-A, aMPV-B, and aMPV-C real-time RT-PCR at ISU VDL from January to November 2024 were included for analysis in this study.

### Nucleic Acid Extraction

Nucleic acids were extracted from samples using a MagMAX Pathogen RNA/DNA Kit (Thermo Fisher Scientific, Waltham, MA, USA) and a Kingfisher Apex instrument (Thermo Fisher Scientific) following the manufacturer's instructions. One hundred microliters of the sample was used for extraction, and nucleic acid was eluted into 90 μL elution buffer. Before nucleic acid extraction, an internal positive control XIPC RNA (1×10⁴ copies per extraction) was added to the extraction lysis buffer. As a result, the extracted nucleic acid from each sample was expected to contain both the XIPC RNA and the target pathogen nucleic acid.

### Avian Metapneumovirus Subtypes A, B, and C Real-Time RT-PCR

Previously described aMPV-A, aMPV-B, and aMPV-C real-time RT-PCR assays, targeting the G gene of aMPV-A and aMPV-B and the SH gene of aMPV-C, were used in this study to test for the presence of aMPV-A, -B, and -C in clinical samples and cell culture isolates. In brief, each individual PCR was set up in a 20 μL reaction: 5 μL of TaqMan® Fast 1-Step Master Mix (Thermo Fisher Scientific), 0.8 μL of forward primer at 10 μM, 0.8 μL of reverse primer at 10 μM, 0.16 μL of probe at 25 μM, 0.2 μL XIPC forward primer at 20 μM, 0.2 μL of XIPC reverse primer at 20 μM, 0.15 μL of XIPC probe at 10 μM, 4.69 μL nuclease-free water, and 5 μL nucleic acid extract. Amplification reactions were performed on an ABI 7500 Fast Instrument (Thermo Fisher Scientific) with the following PCR conditions: one cycle of 50° C. for 5 min, one cycle of 95° C. for 20 sec, and 40 cycles of 95° C. for 15 sec and 60° C. for 30 sec. The analysis was performed using an automatic baseline, probe detector at a threshold of 0.1, and an XIPC detector (Cy5) at 10% of the maximum height of the sigmoid amplification curve. Samples with Ct<37 were considered PCR-positive and samples with Ct≥37 were considered PCR-negative for aMPV.

### Cell Lines, Embryonated Chicken Eggs, and Primary Chicken Embryo Cells

African green monkey kidney cell line Vero (ATCC CCL-81) and chicken embryo fibroblast cell line UMNSAH/DF-1 (ATCC CRL-3586) were cultured and maintained in Dulbecco's Modified Eagle Medium (DMEM) supplemented with 10% fetal bovine serum (FBS), 2 mM L-glutamine, 100 unit/ml penicillin, 100 μg/ml streptomycin, and 0.25 μg/ml amphotericin.

Specific pathogen-free embryonated chicken eggs (ECE) at 9 days of age were ordered from National Veterinary Service Laboratory, the United States Department of Agriculture and incubated at 37° C. with passive humidity.

Primary chicken embryo fibroblast (CEF) cells were prepared from embryonated chicken eggs at 9-10 days of age. Briefly, the heads, wings, legs, and internal organs of the prechilled embryos were removed, and the remaining tissues were washed three times with phosphate-buffered saline (PBS, pH 7.4). The embryos were then cut into 1 cm pieces, washed three times with PBS, and treated with a 0.25% trypsin solution at 37° C. for 10-15 min until the tissue pieces became fluffy. After digestion, the trypsin was removed, and the tissue cells were washed twice with PBS and once with the cell culture media (DMEM supplemented with 10% FBS, 2 mM L-glutamine, 100 unit/ml penicillin, 100 μg/ml streptomycin, and 0.25 μg/ml amphotericin). The cells were then resuspended in cell culture media and filtered through a funnel containing 8 layers of gauze three times. Subsequently, the cell suspensions were collected and cells from multiple embryos could be pooled. The prepared cell suspensions were either frozen in liquid nitrogen for future use or freshly aliquoted into 24-well culture plates (1 ml per well) and cultured at 37° C. with 5% CO₂. Typically, a monolayer with over 90% confluence was achieved within 24 hours. A single chicken embryo could yield primary fibroblast cells to populate up to 150 wells of 24-well plates.

Primary chicken embryo lung (CEL) cells were prepared from 19-day-old embryonated chicken eggs. The 9-day-old embryonated chicken eggs ordered from NVSL were incubated in an egg incubator until they reached 19 days old. The lung tissues from prechilled embryos were collected and washed three times with PBS. Subsequently, the lung tissues were cut into 1 cm pieces, washed three times with PBS, and treated with a 0.25% trypsin solution at 37° C. for 25-30 min until the tissue pieces became fluffy. After digestion, the trypsin was removed, and the tissue cells were washed twice with PBS and once with the cell culture media. The cells were then resuspended in cell culture media and filtered through a funnel containing 8 layers of gauze three times. Subsequently, the cell suspensions were collected and cells from multiple embryos could be pooled. The prepared cell suspensions were either frozen in liquid nitrogen for future use or freshly aliquoted into 24-well culture plates (1 ml per well) and cultured at 37° C. with 5% CO₂. Typically, a monolayer with over 90% confluence was achieved in 4-5 days. A single chicken embryo could yield primary lung cells to populate up to 48 wells of 24-well plates.

Primary Isolation of aMPV from Clinical Samples

Isolation of aMPV-A and aMPV-B from PCR-positive clinical samples was attempted in the Vero cell line, UMNSAH/DF-1 cell line, 9-day-old ECE, primary CEF cells and/or primary CEL cells. The clinical samples were first filtered through 0.22 μm syringe filter.

The cell lines or primary cells cultured in 24-well plates were washed once with PBS and once with the washing media (DMEM supplemented with 2 mM L-glutamine, 100 unit/ml penicillin, 100 μg/ml streptomycin, and 0.25 μg/ml amphotericin). Then, the filtered samples were inoculated into cells (0.1-0.2 ml per well) and incubated at 37° C. with 5% CO₂ for 1.5 hours. The inoculum was removed and the virus isolation media (DMEM supplemented with 2% FBS, 2 mM L-glutamine, 100 unit/ml penicillin, 100 μg/ml streptomycin, and 0.25 μg/ml amphotericin) was added (1 ml per well). The plates were incubated at 37° C. with 5% CO₂. The development of cytopathic effects (CPE) was checked daily. When ~70-80% of viral CPEs were observed, the plates were subjected to two freeze-thaw cycles before harvesting. If no CPE was observed after 4-5 days, the plates were subjected to two freeze-thaw cycles before harvesting. The cell lysates were centrifuged at 2,000 rpm for 10 min and the supernatants were harvested. The supernatants were tested by aMPV-A, -B, and -C real-time RT-PCR as described above. Virus isolation (VI) was performed through three passages (P0-P2) in Vero cells or UMNSAH/DF-1 cells and five passages (P0-P4) in primary CEF and CEL cells. In Vero and UMNSAH/DF-1 cells, samples were deemed VI-negative if no CPE was observed after three passages and the third-passage cell culture supernatant tested PCR-negative. In primary CEF and CEL cells, samples were considered VI-negative if no CPE was observed after five passages and the fifth-passage cell culture supernatant was PCR-negative. In contrast, if CPE was observed during the course of five passages and the fifth-passage cell culture supernatant tested PCR-positive for aMPV with Ct≤20, the samples were considered VI-positive in primary CEF and/or CEL cells.

For VI in embryonated chicken eggs, 0.2 ml of each sample was inoculated into the allantoic cavity of three 9-day-old ECE, followed by incubation at 37° C. with passive humidity for 3-4 days. After chilling the eggs at 4° C. for approximately two hours, the allantoic fluids were harvested and centrifuged at 2,000 rpm for 10 min. The supernatants were tested by aMPV-A, -B, and -C real-time RT-PCR as described above. For each sample, three passages of VI were conducted. The samples were considered VI-negative if egg fluids from the third passage were negative by aMPV PCR.

Serial Passages of aMPV-A and aMPV-B Isolates in Cell Culture

Two aMPV-A isolates (USA/IA55601-6/2024 and USA/IA56509-5/2024) were serially passaged in primary CEL cells and two aMPV-B isolates (USA/NC20487-GA/2024 and USA/NC23734-GA/2024) were serially passaged in primary CEF cells for 10 passages (P0-P9). The cell culture supernatants at each passage were tested by aMPV-A, -B, and -C real-time RT-PCR.

Two aMPV-A isolates (USA/IA55601-6/2024 and USA/IA56509-5/2024) at P4 obtained from primary CEL cells were inoculated into Vero cells and serially propagated for 10 passages (P1-P10). Similarly, aMPV-B isolate USA/NC20487-GA/2024 at P5 and USA/NC23734-GA/2024 isolate at P6 obtained from primary CEF cells were inoculated into Vero cells and serially propagated for 10 passages (P1-P10). The cell culture supernatants at each passage were tested by aMPV-A, -B, and -C real-time RT-PCR and titrated in Vero cells. Immunofluorescence staining was also performed on aMPV-inoculated Vero cells to verify virus replication.

Titration of aMPV-A and aMPV-B Isolates in Vero Cells

The infectious titers of aMPV-A and aMPV-B isolates adapted in Vero cells (P1-P10) were determined in Vero cells. Each virus isolate at each passage was 10-fold serially diluted and 100 μl of each dilution was inoculated into Vero cells grown in 96-well plates with triplicate wells per dilution. CPE was recorded for each well daily up to 4-5 days post inoculation. Immunofluorescence staining was conducted to verify virus replication. Virus titers were calculated according to the Reed and Muench method and expressed as TCID₅₀/ml.

### Immunofluorescence Staining

Mock-infected and aMPV-infected Vero cells cultured in 96-well plates were fixed with 80% cold acetone. After air drying, the plates were rinsed with 100 μL of PBS and incubated with 100 μL of 40-fold diluted turkey serum samples for one hour at 37° C. Three types of turkey serum samples were used: (i) aMPV ELISA antibody-negative turkey serum, (ii) aMPV-A ELISA antibody-positive serum from a turkey that was PCR-positive only for aMPV-A, and (iii) aMPV-B ELISA antibody-positive serum from a turkey that was PCR-positive only for aMPV-B. The avian metapneumovirus ELISA test (IDEXX, Westbrook, ME, USA) was performed according to the manufacturer's protocol.

After incubation, the plates were washed three times with PBS (200 μL/well). Then, 50 μL of 400-fold diluted goat anti-turkey IgG conjugated to fluorescein isothiocyanate (Thermo Fisher Scientific) was added to each well. Following a 45-min incubation at 37° C., the plates were washed three more times with PBS (200 μL/well). The plates were read under a fluorescence microscope.

### Whole-Genome Sequencing and Sequence Analysis

The aMPV-A isolate USA/IA55601-6/2024 at P1 and P9 in primary CEL cells and at P4 and P10 in Vero cells, the aMPV-A isolate USA/IA56509-5/2024 at P3 and P9 in primary CEL cells and at P4 and P10 in Vero cells, the aMPV-B isolate USA/NC20487-GA/2024 at P3 and P9 in primary CEF cells and at P4 and P10 in Vero cells, and the aMPV-B isolate USA/NC23734-GA/2024 at P1 and P9 in primary CEF cells and at P4 and P10 in Vero cells were subjected to whole genome sequencing via next-generation sequencing (NGS) on Illumina MiSeq platform following previously described procedures for other RNA viruses.

In addition to the aMPV-A and aMPV-B sequences determined in this study, 46 whole genome sequences of aMPV retrieved from GenBank on Jan. 8, 2025 were included for analysis. These 46 sequences included 13 aMPV-A, 18 aMPV-B, 14 aMPV-C, and one aMPV-D, and their information is summarized in supplemental Table S1. Nucleotide identities were calculated by the MAFFT alignment of MegAlign Pro 17 program in the DNASTAR Lasergene 17 software. For phylogenetic analysis, sequences were first aligned using the progressive method (FFT-NS-2) in MAFFT v7.407, and then the phylogenetic trees based on aMPV G gene nucleotides and complete genome sequences were inferred from the alignment results using maximum likelihood with a stochastic algorithm in IQ-TREE v2.2.2.6 with 1,000 bootstrap replicates.

### Results

Summary of aMPV PCR Results at ISU VDL from January Through November 2024

The 2,204 clinical samples tested by aMPV-A, -B, and -C real-time RT-PCR at ISU VDL from January to November 2024 included 1,158 turkey samples, 936 chicken samples, and 110 other breed samples. All samples were PCR-negative for aMPV-C. Among 1,158 turkey samples, 591 (51.04%) were PCR-positive for aMPV-A and/or aMPV-B (35.41% aMPV-A positive only, 14.25% aMPV-B positive only, and 1.38% positive for both aMPV-A and aMPV-B) (TABLE 1). In contrast, only 146/936 (15.6%) of chicken samples were PCR-positive for aMPV-A and/or aMPV-B (9.72% aMPV-A positive only, 5.2% aMPV-B positive only, and 0.64% positive for both aMPV-A and aMPV-B) (TABLE 1). Regarding 110 samples of other breeds, only two samples were aMPV-A PCR-positive (TABLE 1). The number of samples and their PCR-positive rates were further analyzed at different age categories and are summarized in Table 1. aMPV-A and aMPV-B were detected across different ages of turkeys and chickens with varying positive rates. For turkeys, more clinical samples in the age of 3-24 weeks were received and relatively higher positive rates were detected in this age range compared to other age categories. For chickens, most of the received samples were in the age of ≥24 weeks and no clear trends of positive rates were observed across different age categories.

TABLE 1
Summary of aMPV PCR results at ISU VDL from January to November 2024.

| | | Number of samples (% at each age category) | | | | | |
|---|---|---|---|---|---|---|---|
| | aMPV RT- | | | 3 to <8 | 8 to <24 | ≥24 | Unknown |
| Breed | PCR result | All age | <3 weeks | weeks | weeks | weeks | age |
| Turkey | aMPV-A | 410 | 38 | 112 | 208 | 0 | 52 |
| | Pos only | (35.41%) | (22.49%) | (36.60%) | (39.62%) | | (41.94%) |
| | aMPV-B | 165 | 7 | 65 | 75 | 11 | 7 |
| | Pos only | (14.25%) | (4.14%) | (21.24%) | (14.29%) | (32.35%) | (5.64%) |
| | aMPV-A | 16 | 0 | 11 | 5 | 0 | 0 |
| | and -B Pos | (1.38%) | | (3.60%) | (0.95%) | | |
| | aMPV-A | 567 | 124 | 118 | 237 | 23 | 65 |
| | and -B Neg | (48.96%) | (73.37%) | (38.56%) | (45.14%) | (67.65%) | (52.42%) |
| | Subtotal | 1158 | 169 | 306 | 525 | 34 | 124 |
| | | (100%) | (100%) | (100%) | (100%) | (100%) | (100%) |
| Chicken | aMPV-A | 91 | 2 | 7 | 9 | 73 | 0 |
| | Pos only | (9.72%) | (16.67%) | (20.00%) | (12.33%) | (9.45%) | |
| | aMPV-B | 49 | 0 | 2 | 6 | 27 | 14 |
| | Pos only | (5.24%) | | (5.71%) | (8.22%) | (3.49%) | (32.56%) |
| | aMPV-A | 6 | 0 | 1 | 4 | 1 | 0 |
| | and -B Pos | (0.64%) | | (2.86%) | (5.48%) | (0.13%) | |
| | aMPV-A | 790 | 10 | 25 | 54 | 672 | 29 |
| | and -B Neg | (84.40%) | (83.33%) | (71.43%) | (73.97%) | (86.93%) | (67.44%) |
| | Subtotal | 936 | 12 | 35 | 73 | 773 | 43 |
| | | (100%) | (100%) | (100%) | (100%) | (100%) | (100%) |
| Other | aMPV-A | 2 | 0 | 0 | 0 | 0 | 2 |
| breed | Pos only | (1.82%) | | | | | (4.76%) |
| | aMPV-B | 0 | 0 | 0 | 0 | 0 | 0 |
| | Pos only | | | | | | |
| | aMPV-A | 0 | 0 | 0 | 0 | 0 | 0 |
| | and -B Pos | | | | | | |
| | aMPV-A | 108 | 0 | 5 | 0 | 63 | 40 |
| | and -B Neg | (98.18%) | | (100%) | | (100%) | (95.24%) |
| | Subtotal | 110 | 0 | 5 | 0 | 63 | 42 |
| | | (100%) | | (100%) | | (100%) | (100%) |
| All | Total | 2204 | 181 | 346 | 598 | 870 | 209 |

Note:
All samples tested negative for aMPV-C by PCR.

The state distributions of tested clinical samples are summarized in TABLE 2. Among the turkey samples submitted to the ISU VDL for aMPV testing, the top three states of origin were Iowa, Ohio, and North Carolina. In Iowa, 228/466 (48.93%) and 22/466 (4.72%) of turkey samples were aMPV-A and aMPV-B PCR-positive, respectively, clearly indicating that aMPV-A was more frequently detected than aMPV-B in Iowa turkeys. In Ohio, similar rates of turkey samples were PCR-positive for aMPV-A (78/243; 32.10%) and aMPV-B (61/243; 25.10%). In North Carolina, more turkey samples were positive for aMPV-B (50/164; 30.49%) than that for aMPV-A (1/50; 2%). Among the chicken samples submitted to the ISU VDL for aMPV testing, the top states of origin were North Carolina, Indiana, and Arkansas, followed by Missouri, Oklahoma, Iowa, Maryland, Tennessee, and Ohio. More chicken samples were positive for aMPV-A than aMPV-B in Arkansas, Missouri, and Oklahoma, a similar number of chicken samples were positive for aMPV-A and aMPV-B in Indiana and Ohio, and more chicken samples were positive for aMPV-B than aMPV-A in North Carolina.

TABLE 2
State distributions of clinical samples tested for aMPV by PCR at ISU VDL from January to November 2024.

| | Number of turkey samples | | | Number of chicken samples | | | Number of other breed samples | | | |
|---|---|---|---|---|---|---|---|---|---|---|
| | | aMPV-A | aMPV-B | | aMPV-A | aMPV-B | | aMPV-A | aMPV-B | |
| State | Subtotal | PCR-Pos | PCR-Pos | Subtotal | PCR-Pos | PCR-Pos | Subtotal | PCR-Pos | PCR-Pos | Total |
| Arkansas | 0 | 0 | 0 | 123 | 31 | 1 | 0 | 0 | 0 | 123 |
| California | 17 | 9 | 0 | 13 | 7 | 1 | 0 | 0 | 0 | 30 |
| Colorado | 0 | 0 | 0 | 2 | 0 | 0 | 0 | 0 | 0 | 2 |
| Georgia | 0 | 0 | 0 | 12 | 0 | 6 | 0 | 0 | 0 | 12 |
| Iowa | 466 | 228 | 22 | 69 | 1 | 6 | 101 | 0 | 0 | 636 |
| Illinois | 3 | 0 | 0 | 5 | 1 | 0 | 0 | 0 | 0 | 8 |
| Indiana | 48 | 23 | 9 | 167 | 7 | 7 | 0 | 0 | 0 | 215 |
| Maryland | 0 | 0 | 0 | 54 | 0 | 0 | 0 | 0 | 0 | 54 |
| Michigan | 15 | 5 | 10 | 10 | 2 | 3 | 0 | 0 | 0 | 25 |
| Minnesota | 41 | 6 | 15 | 4 | 0 | 0 | 2 | 0 | 0 | 47 |
| Missouri | 1 | 0 | 0 | 94 | 14 | 1 | 0 | 0 | 0 | 95 |
| Montana | 0 | 0 | 0 | 0 | 0 | 0 | 1 | 0 | 0 | 1 |
| North | 164 | 1 | 50 | 184 | 0 | 11 | 0 | 0 | 0 | 348 |
| Carolina | | | | | | | | | | |
| Ohio | 243 | 78 | 61 | 50 | 8 | 12 | 0 | 0 | 0 | 293 |
| Oklahoma | 0 | 0 | 0 | 72 | 25 | 0 | 0 | 0 | 0 | 72 |
| South | 1 | 0 | 1 | 12 | 0 | 0 | 0 | 0 | 0 | 13 |
| Carolina | | | | | | | | | | |
| South | 2 | 0 | 0 | 0 | 0 | 0 | 0 | 0 | 0 | 2 |
| Dakota | | | | | | | | | | |
| Tennessee | 0 | 0 | 0 | 53 | 0 | 5 | 0 | 0 | 0 | 53 |
| Texas | 0 | 0 | 0 | 1 | 0 | 0 | 0 | 0 | 0 | 1 |
| Virginia | 0 | 0 | 0 | 3 | 0 | 1 | 0 | 0 | 0 | 3 |
| Wisconsin | 7 | 5 | 0 | 3 | 0 | 1 | 0 | 0 | 0 | 10 |
| West | 11 | 0 | 0 | 0 | 0 | 0 | 0 | 0 | 0 | 11 |
| Virginia | | | | | | | | | | |
| Unknown | 139 | 71 | 13 | 5 | 1 | 0 | 6 | 2 | 0 | 150 |
| Total | 1158 | 426 | 181 | 936 | 97 | 55 | 110 | 2 | 0 | 2204 |

For both turkeys and chickens, the top two specimen types submitted to the ISU VDL for aMPV PCR testing were oropharyngeal swabs and tracheal swabs (TABLE 3). In turkeys, 52.15% of oropharyngeal swabs and 53.39% of tracheal swabs were PCR-positive for aMPV-A and/or aMPV-B. In contrast, in chickens, 14.56% of oropharyngeal swabs and 20.40% of tracheal swabs were PCR-positive for aMPV-A and/or aMPV-B. In both turkeys and chickens, samples had varying aMPV PCR Ct ranges regardless of specimen types.

TABLE 3
Specimen types of clinical samples tested for aMPV by PCR at ISU VDL from January to November 2024.

| | Turkey: number of samples | | | | | Chicken: number of samples | | | | |
|---|---|---|---|---|---|---|---|---|---|---|
| | by status of aMPV PCR | | | | | by status of aMPV PCR | | | | |
| | aMPV- | aMPV- | aMPV-A | | | aMPV- | aMPV- | aMPV-A | | |
| | A Pos | B Pos | and -B | aMPV-A | | A Pos | B Pos | and -B | aMPV-A | |
| | only (Ct | only (Ct | Pos (Ct | and -B | | only (Ct | only (Ct | Pos (Ct | and -B | |
| Specimen type | range) | range) | range) | Neg [%] | Total | range) | range) | range) | Neg [%] | Total |
| Oropharyngeal | 265 | 105 | 6 | 345 | 721 | 41 | 27 | 1 | 405 | 474 |
| swab | (17.5- | (16.5- | (16.2- | [47.85%] | | (18.1- | (17.7- | (27.8- | [85.44%] | |
| | 36.9) | 36.9) | 36.8) | | | 36.6) | 36.9) | 29.3) | | |
| Trachea | 8 | 5 | 0 | 28 | 41 | 1 | 2 | 3 | 16 | 22 |
| | (20.8- | (18.6- | | [68.29%] | | (31.1) | (20.9- | (25.4- | [72.73%] | |
| | 34.2) | 31.4) | | | | | 29.2) | 36.7) | | |
| Tracheal swab | 92 | 33 | 9 | 117 | 251 | 38 | 12 | 1 | 199 | 250 |
| | (19.1- | (20.1- | (19.1- | [46.61%] | | (18.4- | (20.7- | (22.3- | [79.60%] | |
| | 36.9) | 36.6) | 36.1) | | | 36.5) | 35.5) | 22.5) | | |
| Tracheal/ | 18 | 2 | 0 | 14 | 34 | 0 | 1 | 0 | 3 | 4 |
| Oropharyngeal | (21.6- | (31.3- | | [41.18%] | | | (29.1) | | [75%] | |
| swab | 35.2) | 31.7) | | | | | | | | |
| Turbinates | 8 | 6 | 1 | 21 | 36 | 0 | 0 | 0 | 1 | 1 |
| | (21.5- | (20.4- | (19.2- | [58.33%] | | | | | [100%] | |
| | 30.7) | 36.4) | 21.5) | | | | | | | |
| Other | 19 | 14 | 0 | 42 | 75 | 11 | 7 | 1 | 166 | 185 |
| | (19.5- | (13.1- | | [56.00%] | | (24.2- | (33.4- | (18.5- | [89.73%] | |
| | 36.2) | 35.8) | | | | 36.5) | 36.7) | 22.4) | | |
| Total | 410 | 165 | 16 | 567 | 1158 | 91 | 49 | 6 | 790 | 936 |
| | | | | [48.96%] | | | | | [84.40%] | |

Primary Isolation of aMPV-A and aMPV-B from Clinical Samples

Initially, 11 aMPV-A PCR-positive samples in the Ct range of 18.9-31.1 and 36 aMPV-B PCR-positive samples in the Ct range of 16.5-34.1 were directly subjected to VI attempts in the continuous Vero cell line. The cell lysates at three passages (P0-P2) were tested by aMPV-A and aMPV-B real-time RT-PCR. Due to the absence of CPE and the negative PCR results on the third-passage cell lysates, VI was considered negative for these samples in the Vero cell line.

Subsequently, 12 aMPV-A PCR-positive samples in the Ct range of 18.9-24.8 and 12 aMPV-B PCR-positive samples in the Ct range of 16.5-28 were directly inoculated into the continuous UMNSAH/DF-1 cell line for VI attempts. No CPE was observed during three passages (P0-P2) and the third-passage cell lysates tested PCR-negative for aMPV. Therefore, VI was considered unsuccessful for these samples in the UMNSAH/DF-1 cell line.

Three aMPV-A PCR-positive samples in the Ct range of 18.9-25.1 and two aMPV-B PCR-positive samples in the Ct range of 20.3-22.3 were also directly inoculated into 9-day-old ECE via allantoic cavity route for VI attempts. After three passages (P0-P2), the harvested egg fluids were negative by aMPV-A and aMPV-B PCR and VI was considered negative.

Next, virus isolation was attempted in primary CEF cells and/or primary CEL cells prepared. Forty-nine aMPV-A PCR-positive samples in the Ct range of 17.5-32.0 (47 samples positive for aMPV-A only and two samples positive for both aMPV-A and aMPV-B) were subjected to VI attempts in primary cells. As shown in TABLE 4, four samples (USA/IA55601-6/2024, USA/IA56509-5/2024, USA/OH44164-1/2024, and USA/IN39902-1/2024) were aMPV-A VI-positive in either primary CEF cells or primary CEL cells or both. Similarly, 42 aMPV-B PCR-positive samples in the Ct range of 16.5-33.8 (40 samples positive for aMPV-B only and two samples positive for both aMPV-A and aMPV-B) were subjected to VI attempts in primary cells. Three samples (USA/NC23734-GA/2024, USA/NC20487-GA/2024, and USA/NC39727-GB/2024) were aMPV-B VI-positive in either primary CEF cells alone or in both primary CEF and primary CEL cells (TABLE 5). Regarding the two samples that were PCR-positive for both aMPV-A and aMPV-B (USA/OH44164-1/2024 and USA/OH46642-1/2024), the sample USA/OH44164-1/2024 was aMPV-A VI-positive and aMPV-B VI-negative whereas the sample USA/OH46642-1/2024 was both aMPV-A and aMPV-B VI-negative. For four aMPV-A and three aMPV-B VI-positive samples, CPE was not apparent during P0-P2; however, from P3 onward, CPE, characterized by rounded and floating cells, appeared in primary CEF and CEL cells. The specimen types of these seven VI-positive samples are oropharyngeal swab, tracheal swab, or trachea (TABLE 4 and TABLE 5).

Two aMPV-A isolates (USA/IA55601-6/2024 and USA/IA56509-5/2024) and two aMPV-B isolates (USA/NC20487-GA/2024 and USA/NC23734-GA/2024) were selected for 10 serial passages (P0-P9) in primary CEL cells and primary CEF cells, respectively. As shown in TABLE 6, two aMPV-A and two aMPV-B isolates grew efficiently upon serial passages in primary CEL and primary CEF cells, respectively, as evidenced by consistent low PCR Ct values during P2-P9.

TABLE 4
Virus isolation attempts of aMPV-A in primary chicken embryo fibroblast and lung cells

| | | | | PCR Ct on | VI in | |
|---|---|---|---|---|---|---|
| | | | | clinical | primary | VI in |
| | | | | sample/ | fibroblast | primary |
| | ID | Breed | Sample | subtype | cells | lung cells |
| 1 | USA/IA55601-4/2024 | Turkey | Oropharyngeal swab | 17.5/A | Neg | Neg |
| 2 | USA/IA55601-5/2024 | Turkey | Oropharyngeal swab | 17.6/A | Neg | Neg |
| 3 | USA/IA55601-6/2024 | Turkey | Oropharyngeal swab | 17.6/A | Neg | Pos |
| 4 | USA/CA13309-53/2024 | Turkey | Nasal turbinates | 18.9/A | Neg | Neg |
| 5 | USA/IA55601-3/2024 | Turkey | Oropharyngeal swab | 18.9/A | Neg | ND* |
| 6 | USA/IA56509-5/2024 | Turkey | Oropharyngeal swab | 18.9/A | Neg | Pos |
| 7 | USA/IA56509-1/2024 | Turkey | Oropharyngeal swab | 19.4/A | Neg | Neg |
| 8 | USA/CA13309-55/2024 | Turkey | Nasal turbinates | 20.1/A | Neg | Neg |
| 9 | USA/IA56509-2/2024 | Turkey | Oropharyngeal swab | 20.1/A | Neg | ND |
| 10 | USA/CA26913/2024 | Turkey | Sinus tissue | 20.4/A | Neg | Neg |
| 11 | USA/42644-2/2024 | Turkey | Oropharyngeal swab | 21.2/A | Neg | Neg |
| 12 | USA/37711-1/2024 | Turkey | Oropharyngeal swab | 21.6/A | Neg | Neg |
| 13 | USA/42659-2/2024 | Turkey | Oropharyngeal swab | 22/A | Neg | Neg |
| 14 | USA/IA56509-3/2024 | Turkey | Oropharyngeal swab | 22.0/A | Neg | ND |
| 15 | USA/IA66621-6/2024 | Turkey | Oropharyngeal swab | 22.0/A | Neg | Neg |
| 16 | USA/IA55601-8/2024 | Turkey | Oropharyngeal swab | 22.4/A | Neg | ND |
| 17 | USA/IA56509-4/2024 | Turkey | Oropharyngeal swab | 22.4/A | Neg | ND |
| 18 | USA/OH44164-1/2024 | Chicken | Tracheal swab | 22.5/A; | Pos | Neg |
| | | | | 22.3/B | (aMPV-A) | |
| 19 | USA/IA66621-5/2024 | Turkey | Oropharyngeal swab | 22.5/A | Neg | Neg |
| 20 | USA/37711-2/2024 | Turkey | Oropharyngeal swab | 22.6/A | Neg | Neg |
| 21 | USA/42655-2/2024 | Turkey | Oropharyngeal swab | 22.7/A | Neg | Neg |
| 22 | USA/IA66621-2/2024 | Turkey | Oropharyngeal swab | 22.7/A | Neg | Neg |
| 23 | USA/42659-1/2024 | Turkey | Oropharyngeal swab | 23/A | Neg | ND |
| 24 | USA/IA66621-4/2024 | Turkey | Oropharyngeal swab | 23.0/A | Neg | Neg |
| 25 | USA/37706-1/2024 | Turkey | Oropharyngeal swab | 23.1/A | Neg | Neg |
| 26 | USA/IA66621-1/2024 | Turkey | Oropharyngeal swab | 23.2/A | Neg | Neg |
| 27 | USA/IA55601-7/2024 | Turkey | Oropharyngeal swab | 23.4/A | Neg | ND |
| 28 | USA/42644-1/2024 | Turkey | Oropharyngeal swab | 23.8/A | Neg | Neg |
| 29 | USA/OH46642-1/2024 | Turkey | Tracheal swab | 23.9/A; | Neg | Neg |
| | | | | 25.4/B | | |
| 30 | USA/42643-4/2024 | Turkey | Oropharyngeal swab | 24.1/A | Neg | Neg |
| 31 | USA/42647-1/2024 | Turkey | Oropharyngeal swab | 24.3/A | Neg | Neg |
| 32 | USA/IN39902-1/2024 | Chicken | Oropharyngeal swab | 24.8/A | Pos | Pos |
| 33 | USA/37707-2/2024 | Turkey | Oropharyngeal swab | 24.9/A | Neg | Neg |
| 34 | USA/CA16068-GA/2024 | Turkey | Nasal turbinates | 25.1/A | Neg | Neg |
| 35 | USA/42645-1/2024 | Turkey | Oropharyngeal swab | 25.2/A | Neg | Neg |
| 36 | USA/42651-1/2024 | Turkey | Oropharyngeal swab | 25.3/A | Neg | Neg |
| 37 | USA/42655-1/2024 | Turkey | Oropharyngeal swab | 25.3/A | Neg | Neg |
| 38 | USA/37709-1/2024 | Turkey | Oropharyngeal swab | 25.4/A | Neg | Neg |
| 39 | USA/37710/2024 | Turkey | Oropharyngeal swab | 25.4/A | Neg | Neg |
| 40 | USA/42643-2/2024 | Turkey | Oropharyngeal swab | 25.5/A | Neg | Neg |
| 41 | USA/42654-2/2024 | Turkey | Oropharyngeal swab | 25.5/A | Neg | Neg |
| 42 | USA/42650-1/2024 | Turkey | Oropharyngeal swab | 25.6/A | Neg | Neg |
| 43 | USA/42643-1/2024 | Turkey | Oropharyngeal swab | 26/A | Neg | Neg |
| 44 | USA/37708-1/2024 | Turkey | Oropharyngeal swab | 26.2/A | Neg | Neg |
| 45 | USA/42645-3/2024 | Turkey | Oropharyngeal swab | 26.6/A | Neg | Neg |
| 46 | USA/37708-2/2024 | Turkey | Oropharyngeal swab | 26.9/A | Neg | Neg |
| 47 | USA/37709-2/2024 | Turkey | Oropharyngeal swab | 27.4/A | Neg | Neg |
| 48 | USA/29099-GA/2024 | Turkey | Turbinates | 29.1/A | Neg | ND |
| 49 | USA/OH36408/2024 | Turkey | Oropharyngeal swab | 32.0/A | Neg | Neg |

*ND: Not Done.

TABLE 5
Virus isolation attempts of aMPV-B in primary chicken embryo fibroblast and lung cells.

| | | | | PCR Ct on | VI in | VI in |
|---|---|---|---|---|---|---|
| | | | | clinical | primary | primary |
| | | | | sample/ | fibroblast | lung |
| | ID | Breed | Sample | subtype | cells | cells |
| 1 | USA/IA21041-GA/2024 | Turkey | Oropharyngeal swab | 16.5/B | Neg | Neg |
| 2 | USA/IA61472-GB/2024 | Chicken | Oropharyngeal swab | 17.7/B | Neg | Neg |
| 3 | USA/IA67228-GA/2024 | Turkey | Oropharyngeal swab | 17.7/B | Neg | Neg |
| 4 | USA/IA22319-GA/2024 | Turkey | Oropharyngeal swab | 17.9/B | Neg | ND* |
| 5 | USA/NC58724-7/2024 | Turkey | Oropharyngeal swab | 17.9/B | Neg | Neg |
| 6 | USA/NC61042-GA/2024 | Turkey | Oropharyngeal swab | 18.3/B | Neg | Neg |
| 7 | USA/NC23734-GA/2024 | Turkey | Trachea | 18.6/B | Pos | Neg |
| 8 | USA/NC38371-GA/2024 | Turkey | Oropharyngeal swab | 19.1/B | Neg | Neg |
| 9 | USA/NC20487-GA/2024 | Turkey | Oropharyngeal swab | 19.7/B | Pos | Neg |
| 10 | USA/MI32566-GA/2024 | Turkey | Oropharyngeal swab | 19.9/B | Neg | Neg |
| 11 | USA/NC38172-GA/2024 | Turkey | Oropharyngeal swab | 19.9/B | Neg | ND |
| 12 | USA/OH42503-1/2024 | Turkey | Tracheal swab | 20.1/B | Neg | Neg |
| 13 | USA/MI31490-GA/2024 | Chicken | Oropharyngeal swab | 20.2/B | Neg | ND |
| 14 | USA/NC15504-GA/2024 | Turkey | Oropharyngeal swab | 20.3/B | Neg | Neg |
| 15 | USA/NC39727-GB/2024 | Turkey | Oropharyngeal swab | 21.1/B | Pos | Pos |
| 16 | USA/NC23734-GA/2024 | Turkey | Nasal turbinates | 21.1/B | Neg | Neg |
| 17 | USA/NC23018-GA/2024 | Turkey | Turbinates | 21.5/B | Neg | Neg |
| 18 | USA/OH35792-1/2024 | Turkey | Oropharyngeal swab | 21.7/B | Neg | Neg |
| 19 | USA/OH35792-2/2024 | Turkey | Oropharyngeal swab | 21.7/B | Neg | Neg |
| 20 | USA/NC18440-GA/2024 | Turkey | Trachea | 22.2/B | Neg | Neg |
| 21 | USA/MI30774-GA/2024 | Turkey | Oropharyngeal swab | 22.2/B | Neg | Neg |
| 22 | USA/NC23018/2024 | Turkey | Trachea | 22.3/B | Neg | Neg |
| 23 | USA/OH44164-1/2024 | Chicken | Tracheal swab | 22.5/A; | Pos | Neg |
| | | | | 22.3/B | (aMPV-A) | |
| 24 | USA/OH38585/2024 | Turkey | Oropharyngeal swab | 22.7/B | Neg | Neg |
| 25 | USA/OH44441-1/2024 | Turkey | Tracheal swab | 23.0/B | Neg | Neg |
| 26 | USA/OH37369-1/2024 | Turkey | Oropharyngeal swab | 23.1/B | Neg | Neg |
| 27 | USA/IA21041-GB/2024 | Turkey | Oropharyngeal swab | 23.2/B | Neg | Neg |
| 28 | USA/OH37370-1/2024 | Turkey | Oropharyngeal swab | 23.5/B | Neg | Neg |
| 29 | USA/OH40821-1/2024 | Turkey | Oropharyngeal swab | 23.8/B | Neg | Neg |
| 30 | USA/OH38587-1/2024 | Turkey | Oropharyngeal swab | 24.4/B | Neg | Neg |
| 31 | USA/IA38478-GA/2024 | Turkey | Oropharyngeal swab | 24.5/B | Neg | Neg |
| 32 | USA/OH44162-1/2024 | Chicken | Tracheal swab | 24.7/B | Neg | Neg |
| 33 | USA/MI31490-GB/2024 | Chicken | Oropharyngeal swab | 24.8/B | Neg | ND |
| 34 | USA/OH37372-1/2024 | Turkey | Oropharyngeal swab | 25.3/B | Neg | Neg |
| 35 | USA/OH46642-1/2024 | Turkey | Tracheal swab | 23.9/A; | Neg | Neg |
| | | | | 25.4/B | | |
| 36 | USA/NC39727-GA/2024 | Turkey | Oropharyngeal swab | 25.9/B | Neg | Neg |
| 37 | USA/IN36406/2024 | Turkey | Oropharyngeal swab | 27.9/B | Neg | Neg |
| 38 | USA/IN36405/2024 | Turkey | Oropharyngeal swab | 30.6/B | Neg | Neg |
| 39 | USA/OH37368/2024 | Turkey | Oropharyngeal swab | 30.7/B | Neg | Neg |
| 40 | USA/IN39902-2/2024 | Chicken | Oropharyngeal swab | 32.4/B | Neg | Neg |
| 41 | USA/OH36407/2024 | Turkey | Oropharyngeal swab | 33.0/B | Neg | Neg |
| 42 | USA/OH35686-1/2024 | Turkey | Oropharyngeal swab | 33.8/B | Neg | Neg |

*ND: Not Done.

TABLE 6
PCR testing on serial passages of selected aMPV-A and aMPV-B isolates in primary
chicken embryo cells, with aMPV-A isolates in primary chicken embryo lung
cells and aMPV-B isolates in primary chicken embryo fibroblast cells.

| | | | aMPV-A | aMPV-B | aMPV-C |
|---|---|---|---|---|---|
| Virus isolate | Subtype | Passage | PCR Ct | PCR Ct | PCR Ct |
| USA/IA55601-6/2024 | A | P0 | 20.76 | ≥40 | ≥40 |
| | | P1 | 17.87 | ≥40 | ≥40 |
| | | P2 | 16.83 | ≥40 | ≥40 |
| | | P3 | 16.75 | ≥40 | ≥40 |
| | | P4 | 16.86 | ≥40 | ≥40 |
| | | P5 | 15.79 | ≥40 | ≥40 |
| | | P6 | 15.64 | ≥40 | ≥40 |
| | | P7 | 14.87 | ≥40 | ≥40 |
| | | P8 | 15.93 | ≥40 | ≥40 |
| | | P9 | 15.77 | ≥40 | ≥40 |
| USA/IA56509-5/2024 | A | P0 | 25.53 | ≥40 | ≥40 |
| | | P1 | 22.59 | ≥40 | ≥40 |
| | | P2 | 24.41 | ≥40 | ≥40 |
| | | P3 | 21.68 | ≥40 | ≥40 |
| | | P4 | 18.46 | ≥40 | ≥40 |
| | | P5 | 17.63 | ≥40 | ≥40 |
| | | P6 | 15.98 | ≥40 | ≥40 |
| | | P7 | 15.40 | ≥40 | ≥40 |
| | | P8 | 17.00 | ≥40 | ≥40 |
| | | P9 | 16.31 | ≥40 | ≥40 |
| USA/NC20487-GA/2024 | B | P0 | ≥40 | ≥40 | ≥40 |
| | | P1 | ≥40 | 36.18 | ≥40 |
| | | P2 | ≥40 | 20.66 | ≥40 |
| | | P3 | ≥40 | 16.89 | ≥40 |
| | | P4 | ≥40 | 15.24 | ≥40 |
| | | P5 | ≥40 | 13.14 | ≥40 |
| | | P6 | ≥40 | 12.65 | ≥40 |
| | | P7 | ≥40 | 11.00 | ≥40 |
| | | P8 | ≥40 | 11.55 | ≥40 |
| | | P9 | ≥40 | 11.71 | ≥40 |
| USA/NC23734-GA/2024 | B | P0 | ≥40 | 24.54 | ≥40 |
| | | P1 | ≥40 | 17.67 | ≥40 |
| | | P2 | ≥40 | 15.46 | ≥40 |
| | | P3 | ≥40 | 14.13 | ≥40 |
| | | P4 | ≥40 | 13.11 | ≥40 |
| | | P5 | ≥40 | 15.45 | ≥40 |
| | | P6 | ≥40 | 15.97 | ≥40 |
| | | P7 | ≥40 | 15.03 | ≥40 |
| | | P8 | ≥40 | 11.93 | ≥40 |
| | | P9 | ≥40 | 12.26 | ≥40 |

Adaptation of aMPV-A and aMPV-B Isolates Obtained in Primary Cells to Grow in Continuous Vero Cell Line

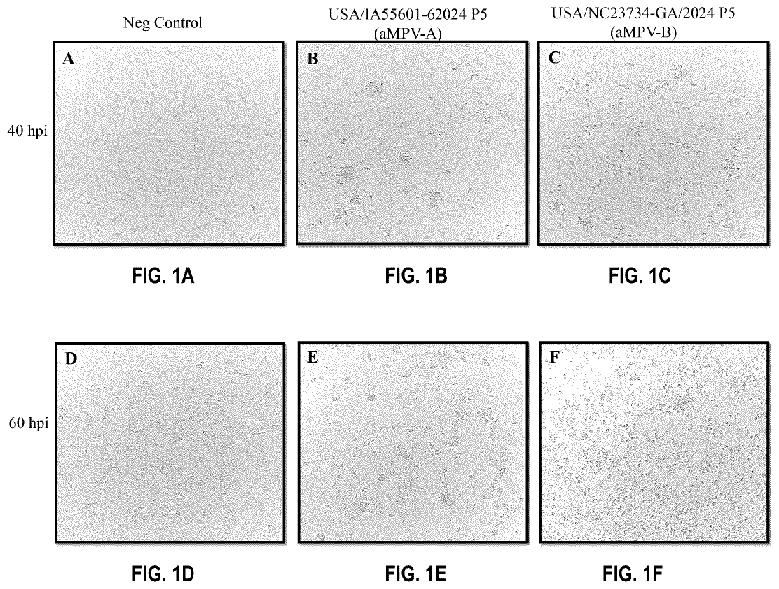

Two aMPV-A isolates (USA/IA55601-6/2024 and USA/IA56509-5/2024) at P4 obtained from primary CEL cells were serially propagated in Vero cells for 10 passages (P1-P10). The aMPV-B isolates USA/NC20487-GA/2024 at P5 and USA/NC23734-GA/2024 isolate at P6 obtained from primary CEF cells were serially propagated in Vero cells for 10 passages (P1-P10). For the aMPV-A isolate USA/IA55601-6/2024, no CPE was observed at P1, mild CPE appeared at P2, and extensive CPE developed from P3 to P10 in Vero cells. Similarly, for the aMPV-A isolate USA/IA56509-5/2024, no CPE was observed at P1 and P2, mild CPE appeared at P3, and extensive CPE was observed during P5-P10 in Vero cells. In contrast, the two aMPV-B isolates (USA/NC20487-GA/2024 and USA/NC23734-GA/2024) exhibited extensive CPE throughout P1-P10 in Vero cells. As exemplified in FIG. 1B, FIG. 1C, FIG. 1E, and FIG. 1F, the characteristic CPE observed in Vero cells inoculated with aMPV-A or aMPV-B included cell rounding and enlargement, with prominent syncytial formation in cells infected with aMPV-A isolate (FIG. 1B and FIG. 1E). In contrast, no CPE was observed in mock-inoculated cells (FIG. 1A, FIG. 1D). Based on the CPE development, it appeared that the aMPV-A isolate USA/IA55601-6/2024 propagated faster than the aMPV-A isolate USA/IA56509-5/2024 while both aMPV-B isolates USA/NC20487-GA/2024 and USA/NC23734-GA/2024 grew efficiently in Vero cells.

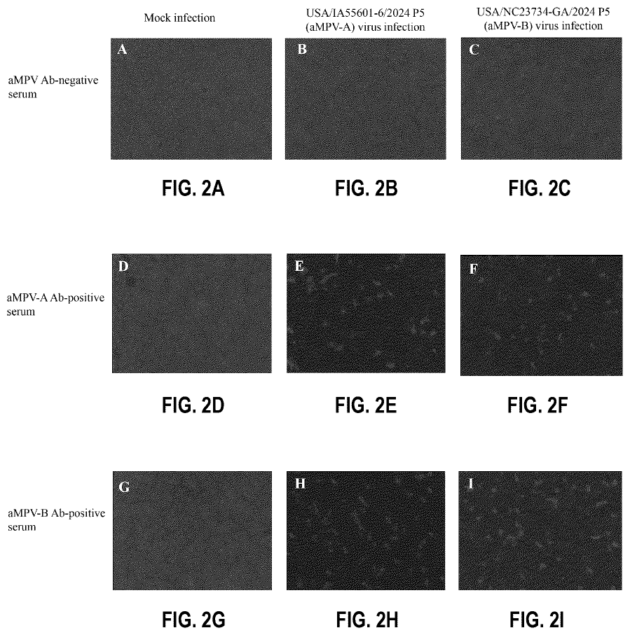

Replication of Vero cell-adapted aMPV-A and aMPV-B isolates was confirmed by immunofluorescence staining, as shown in FIG. 2. aMPV antibody-negative turkey serum showed no specific staining in mock-infected, aMPV-A virus-infected, or aMPV-B virus-infected Vero cells (FIG. 2A-C). aMPV-A antibody-positive turkey serum did not stain mock-infected cells (FIG. 2D) but stained both aMPV-A virus-infected (FIG. 2E) and aMPV-B virus-infected (FIG. 2F) cells. Similarly, aMPV-B antibody-positive turkey serum did not stain mock-infected cells (FIG. 2G) but stained both aMPV-A virus-infected (FIG. 2H) and aMPV-B virus-infected (FIG. 2I) cells.

The Vero cell-adapted aMPV-A and aMPV-B isolates at P1-P10 were also tested by aMPV real-time RT-PCR. As shown in TABLE 7, P1-P10 of the two aMPV-A isolates and two aMPV-B isolates all had low PCR Ct values, indicating that these virus isolates grew efficiently in Vero cells after adaptation. The two aMPV-B isolates overall had relatively lower Ct values compared to the two aMPV-A isolates during P1-P10 in Vero cells. The aMPV-A isolate USA/IA55601-6/2024 had infectious titers of ~10¹-10³ TCID₅₀/ml at P1 and P2, the aMPV-A isolate USA/IA56509-5/2024 had infectious titers of ~10⁰-10¹ TCID₅₀/ml at P1-P3, but both isolates had titers of ~10⁴-10⁶ TCID₅₀/ml at P4-P10 (TABLE 7), demonstrating that the aMPV-A isolates could be adapted to grow efficiently in Vero cells after a few passages. The two aMPV-B isolates (USA/NC20487-GA/2024 and USA/NC23734-GA/2024) had decent titers from P1 and maintained the titers of ~10⁴-10⁶ TCID₅₀/ml during P1-P10 (TABLE 7), suggesting that these two aMPV-B isolates were readily adapted to grow efficiently in Vero cells.

TABLE 7
PCR testing and TCID₅₀ titers of aMPV-A and aMPV-
B isolates serially passaged in Vero cells.

| | | | aMPV-A | aMPV-B | aMPV-C | TCID₅₀/ml in |
|---|---|---|---|---|---|---|
| Virus isolate | Subtype | Passage | PCR Ct | PCR Ct | PCR Ct | Vero cells |
| USA/IA55601-6/2024 | A | P1 | 16.92 | ≥40 | ≥40 | 1.47 × 10¹ |
| | | P2 | 13.87 | ≥40 | ≥40 | 1.47 × 10³ |
| | | P3 | 15.43 | ≥40 | ≥40 | 1.47 × 10⁴ |
| | | P4 | 13.59 | ≥40 | ≥40 | 3.16 × 10⁴ |
| | | P5 | 14.52 | ≥40 | ≥40 | 6.81 × 10⁵ |
| | | P6 | 15.65 | ≥40 | ≥40 | 6.81 × 10⁵ |
| | | P7 | 14.84 | ≥40 | ≥40 | 3.16 × 10⁵ |
| | | P8 | 15.11 | ≥40 | ≥40 | 3.16 × 10⁶ |
| | | P9 | 15.80 | ≥40 | ≥40 | 3.16 × 10⁶ |
| | | P10 | 15.60 | ≥40 | ≥40 | 6.81 × 10⁵ |
| USA/IA56509-5/2024 | A | P1 | 18.42 | ≥40 | ≥40 | 6.81 × 10⁰ |
| | | P2 | 16.72 | ≥40 | ≥40 | 1.47 × 10¹ |
| | | P3 | 14.87 | ≥40 | ≥40 | 3.16 × 10¹ |
| | | P4 | 12.66 | ≥40 | ≥40 | 1.47 × 10⁴ |
| | | P5 | 14.10 | ≥40 | ≥40 | 6.81 × 10⁴ |
| | | P6 | 15.53 | ≥40 | ≥40 | 3.16 × 10⁵ |
| | | P7 | 14.32 | ≥40 | ≥40 | 1.47 × 10⁵ |
| | | P8 | 15.69 | ≥40 | ≥40 | 3.16 × 10⁶ |
| | | P9 | 15.50 | ≥40 | ≥40 | 1.47 × 10⁶ |
| | | P10 | 15.07 | ≥40 | ≥40 | 6.81 × 10⁵ |
| USA/NC20487-GA/2024 | B | P1 | ≥40 | 11.82 | ≥40 | 3.16 × 10⁵ |
| | | P2 | ≥40 | 12.77 | ≥40 | 6.81 × 10⁴ |
| | | P3 | ≥40 | 13.44 | ≥40 | 1.47 × 10⁵ |
| | | P4 | ≥40 | 12.92 | ≥40 | 1.47 × 10⁶ |
| | | P5 | ≥40 | 13.84 | ≥40 | 1.47 × 10⁵ |
| | | P6 | ≥40 | 14.37 | ≥40 | 1.47 × 10⁶ |
| | | P7 | ≥40 | 13.30 | ≥40 | 3.16 × 10⁵ |
| | | P8 | ≥40 | 12.25 | ≥40 | 3.16 × 10⁶ |
| | | P9 | ≥40 | 12.80 | ≥40 | 1.47 × 10⁶ |
| | | P10 | ≥40 | 12.80 | ≥40 | 1.47 × 10⁶ |
| USA/NC23734-GA/2024 | B | P1 | ≥40 | 9.30 | ≥40 | 3.16 × 10⁵ |
| | | P2 | ≥40 | 11.40 | ≥40 | 6.81 × 10⁴ |
| | | P3 | ≥40 | 12.19 | ≥40 | 1.47 × 10⁴ |
| | | P4 | ≥40 | 10.33 | ≥40 | 1.47 × 10⁶ |
| | | P5 | ≥40 | 12.06 | ≥40 | 3.16 × 10⁵ |
| | | P6 | ≥40 | 12.96 | ≥40 | 3.16 × 10⁵ |
| | | P7 | ≥40 | 11.17 | ≥40 | 6.81 × 10⁵ |
| | | P8 | ≥40 | 10.91 | ≥40 | 1.47 × 10⁶ |
| | | P9 | ≥40 | 13.00 | ≥40 | 3.16 × 10⁶ |
| | | P10 | ≥40 | 12.90 | ≥40 | 1.47 × 10⁶ |

Genetic Stability of aMPV-A and aMPV-B Isolates During Serial Passages in Cell Culture

In order to determine if the aMPV-A and aMPV-B isolates were genetically stable during serial passages in cell culture, the whole genome sequences of two aMPV-A isolates and two aMPV-B isolates at different passages were determined via NGS and the results are summarized in TABLE 8 and TABLE 9. The genomic organization of these viruses was similar to other aMPVs and included the 3′ UTR-N-P-M-F-M2.1-M2.2-SH-G-L-5′ UTR.

The aMPV-A isolate USA/IA55601-6/2024 had a genome length of 13,302 nucleotides across all passages tested (primary CEL cells P1 and P9, Vero cells P4 and P10). Their sequences showed 99.97-99.99% nucleotide identity to each other at the whole genome level, with only five nucleotide substitutions at the positions 959, 1,398, 3,903, 5,964, and 11,118. Of these, the nucleotide change at the position 959 was synonymous, the nucleotide change at the position 5,964 was located in the intergenic region between the SH and G genes, while the other three nucleotide substitutions (positions 1,398, 3,903 and 11,118) resulted in amino acid changes in the P, F, and L proteins (TABLE 8). Similarly, the aMPV-A isolate USA/IA56509-5/2024 also maintained a 13,302-nucleotide genome across all evaluated passages (primary CEL cells P3 and P9, Vero cells P4 and P10), with 99.96-100% nucleotide identity to each other. Five nucleotide substitutions were identified at the positions 2,830, 3,902, 5,939, 8,579, and 10,958. The position 5,939 was located in the intergenic region between the SH and G genes, the nucleotide change at the position 10,958 was synonymous, while the other three nucleotide substitutions (positions 2,830, 3,902 and 8,579) caused amino acid changes in the M, F, and L proteins (TABLE 8).

TABLE 8
Nucleotide and amino acid changes of aMPV-A isolates during serial passages in cell culture.

| | | Nucleotide in^{a} | | | | | Amino acid in^{b} | | | | |
|---|---|---|---|---|---|---|---|---|---|---|---|
| Genome region | | | Primary | | Vero | Vero | | Primary | | Vero | Vero |
| (nucleotide | Encoded | | cells P1 | Primary | cells | cells | | cells P1 | Primary | cells | cells |
| position)^{a} | protein | Position | or P3^{c} | cells P9 | P4 | P10 | Position | or P3^{c} | cells P9 | P4 | P10 |
| aMPV-A USA/IA55601-6/2024 | | | | | | | | | | | |
| 3′ UTR (1-41) | Not applicable | | | | | | | | | | |
| N (42-1217) | Nucleoprotein | 959 | A | G | G | A | | | | | |
| P (1242-2078) | Phosphoprotein | 1398 | C | C | T | C | 53 | P | P | S | P |
| M (2104-2868) | Matrix protein | | | | | | | | | | |
| F (2936-4552) | Fusion protein | 3902- | GAA | GGA | GGA | GGA | 323 | E | G | G | G |
| | | 3904 | | | | | | | | | |
| M2.1 (4579-5139) | M2.1 protein | | | | | | | | | | |
| M2.2 (5096-5317) | M2.2 protein | | | | | | | | | | |
| SH (5370-5894) | Small | | | | | | | | | | |
| | hydrophobic | | | | | | | | | | |
| | protein | | | | | | | | | | |
| Intergenic region | Not applicable | 5964 | G | G | G | A | | | | | |
| G (5977-7152) | Surface | | | | | | | | | | |
| | glycoprotein | | | | | | | | | | |
| L (7238-13252) | Large | 11118 | A | G | G | G | 1294 | K | R | R | R |
| | polymerase | | | | | | | | | | |
| 5′ UTR (13253-13302) | Not applicable | | | | | | | | | | |
| aMPV-A USA/IA56509-5/2024 | | | | | | | | | | | |
| 3′ UTR (1-41) | Not applicable | | | | | | | | | | |
| N (42-1217) | Nucleoprotein | | | | | | | | | | |
| P (1242-2078) | Phosphoprotein | | | | | | | | | | |
| M (2104-2868) | Matrix protein | 2830 | A | A | A | T | 243 | N | N | N | Y |
| F (2936-4552) | Fusion protein | 3902- | GAA | GAA | GAA | AAA | 323 | E | E | E | K |
| | | 3904 | | | | | | | | | |
| M2.1 (4579-5139) | M2.1 protein | | | | | | | | | | |
| M2.2 (5096-5317) | M2.2 protein | | | | | | | | | | |
| SH (5370-5894) | Small | | | | | | | | | | |
| | hydrophobic | | | | | | | | | | |
| | protein | | | | | | | | | | |
| Intergenic region | Not applicable | 5939 | T | T | A | T | | | | | |
| G (5977-7152) | Surface | | | | | | | | | | |
| | glycoprotein | | | | | | | | | | |
| L (7238-13252) | Large | 8579 | T | T | T | C | 448 | F | F | F | L |
| | polymerase | | | | | | | | | | |
| | | 10958 | C | C | C | T | | | | | |
| 5′ UTR (13253-13302) | Not applicable | | | | | | | | | | |

^{a}Nucleotides are numbered according to the respective sequences of aMPV-A USA/IA55601-6/2024 (GenBank PV067037) and aMPV-A USA/IA56509-5/2024 (GenBank PV067041).
^{b}Only nonsynonymous mutations are shown. Amino acids of proteins are numbered according to their locations in the respective proteins.
^{c}Primary chicken embryo lung (CEL) cells P1 for aMPV-A USA/IA55601-6/2024 and primary CEL cells P3 for aMPV-A USA/IA56509-5/2024.

The aMPV-B isolate USA/NC20487-GA/2024 had a genome length of 13,472 nucleotides across all evaluated passages (primary CEF cells P3 and P9, Vero cells P4 and P10), with 99.98-100% nucleotide identity to each other. Among different passages of this virus isolate, only two nucleotide substitutions were identified (positions 3,262 and 4,110), both of them resulting in amino acid changes in the F protein (TABLE 9). The aMPV-B isolate USA/NC23734-GA/2024 had a genome length of 13,474 nucleotides across all passages tested (primary CEF cells P1 and P9, Vero cells P4 and P10), with 99.98-99.99% nucleotide identity to each other at the whole genome level. Three nucleotide substitutions were identified at positions 5,212, 5,295, and 7,041, with the resultant three amino acid changes in the M2.2 and G proteins (TABLE 9). The aMPV-B isolate USA/NC23734-GA/2024 at all of the evaluated passages had an insertion of two nucleotides “AA” in the intergenic region between N and P genes when compared to the aMPV-B isolate USA/NC20487-GA/2024.

TABLE 9
Nucleotide and amino acid changes of aMPV-B isolates during serial passages in cell culture.

| | | Nucleotide in^{a} | | | | | Amino acid in^{b} | | | | |
|---|---|---|---|---|---|---|---|---|---|---|---|
| Genome region | | | Primary | | Vero | Vero | | Primary | | Vero | Vero |
| (nucleotide | | | cells P1 | Primary | cells | cells | | cells P1 | Primary | cells | cells |
| position)^{a} | Encoded protein | Position | or P3^{c} | cells P9 | P4 | P10 | Position | or P3^{c} | cells P9 | P4 | P10 |
| aMPV-B USA/NC20487-GA/2024 | | | | | | | | | | | |
| 3′ UTR (1-38) | Not applicable | | | | | | | | | | |
| N (39-1214) | Nucleoprotein | | | | | | | | | | |
| P (1238-2077) | Phosphoprotein | | | | | | | | | | |
| M (2103-2867) | Matrix protein | | | | | | | | | | |
| F (2928-4544) | Fusion protein | 3262 | T | C | C | C | 112 | V | A | A | A |
| | | 4110 | A | T | T | T | 395 | N | Y | Y | Y |
| M2.1 (4557-5132) | M2.1 protein | | | | | | | | | | |
| M2.2 (5089-5310) | M2.2 protein | | | | | | | | | | |
| SH (5349-5891) | Small hydrophobic | | | | | | | | | | |
| | protein | | | | | | | | | | |
| G (5997-7241) | Surface | | | | | | | | | | |
| | glycoprotein | | | | | | | | | | |
| L (7342-13356) | Large polymerase | | | | | | | | | | |
| 5′ UTR (13357-13472) | Not applicable | | | | | | | | | | |
| aMPV-B USA/NC23734-GA/2024 | | | | | | | | | | | |
| 3′ UTR (1-38) | Not applicable | | | | | | | | | | |
| N (39-1214) | Nucleoprotein | | | | | | | | | | |
| P (1240-2079) | Phosphoprotein | | | | | | | | | | |
| M (2105-2869) | Matrix protein | | | | | | | | | | |
| F (2930-4546) | Fusion protein | | | | | | | | | | |
| M2.1 (4559-5134) | M2.1 protein | | | | | | | | | | |
| M2.2 (5091-5312) | M2.2 protein | 5211- | GAA | GAA | GAA | GGA | 41 | E | E | E | G |
| | | 5213 | | | | | | | | | |
| | | 5295 | T | T | A | T | 69 | Y | Y | N | Y |
| SH (5351-5893) | Small hydrophobic | | | | | | | | | | |
| | protein | | | | | | | | | | |
| G (5999-7243) | Surface | 7041 | C | C | T | C | 348 | P | P | L | P |
| | glycoprotein | | | | | | | | | | |
| L (7344-13358) | Large polymerase | | | | | | | | | | |
| 5′ UTR (13359-13474) | Not applicable | | | | | | | | | | |

^{a}Nucleotides are numbered according to the respective sequences of aMPV-B USA/NC20487-GA/2024 (GenBank PV067045) and aMPV-B USA/NC23734-GA/2024 (GenBank PV067049).
^{b}Amino acids of proteins are numbered according to their locations in the respective proteins.
^{c}Primary chicken embryo fibroblast (CEF) cells P3 for aMPV-B USA/NC20487-GA/2024 and primary CEF cells P1 for aMPV-B USA/NC23734-GA/2024.

Sequence Comparisons with Other aMPV Strains

The aMPV-A and aMPV-B sequences determined in this study were compared to 46 other aMPV-A, -B, -C and -D sequences retrieved from GenBank. At the whole genome level, the two aMPV-A isolates (USA/IA55601-6/2024 and USA/IA56509-5/2024) had 99.93-99.99% nucleotide identity to each other, 99.72-99.92% identity to other U.S. aMPV-A sequences, 97.04-99.61% identity to aMPV-A sequences reported from other countries, 72.46-73.02% identity to aMPV-B sequences, 63.65-63.68% identity to aMPV-C sequences, and 72.95-72.99% identity to the aMPV-D sequence. Similarly, the two aMPV-B isolates (USA/NC20487-GA/2024 and USA/NC23734-GA/2024) had 99.86-99.89% nucleotide identity to each other, 99.81-99.94% identity to other U.S. aMPV-B sequences, 97.29-98.62% identity to aMPV-B sequences reported from other countries, 72.27-72.64% identity to aMPV-A sequences, 63.10-63.50% identity to aMPV-C sequences, and 72.39-72.43% identity to the aMPV-D sequence.

Based on the G gene nucleotide sequences, the two aMPV-A isolates (USA/IA55601-6/2024 and USA/IA56509-5/2024) exhibited 100% nucleotide identity to each other, 99.66% identity to other U.S. aMPV-A sequences, and 94.64-98.98% identity to aMPV-A sequences reported from other countries. Similarly, the two aMPV-B isolates (USA/NC20487-GA/2024 and USA/NC23734-GA/2024) had 99.56-99.65% nucleotide identity to each other, 99.31-99.86% identity to other U.S. aMPV-B sequences, and 93.11-95.87% identity to aMPV-B sequences reported from other countries.

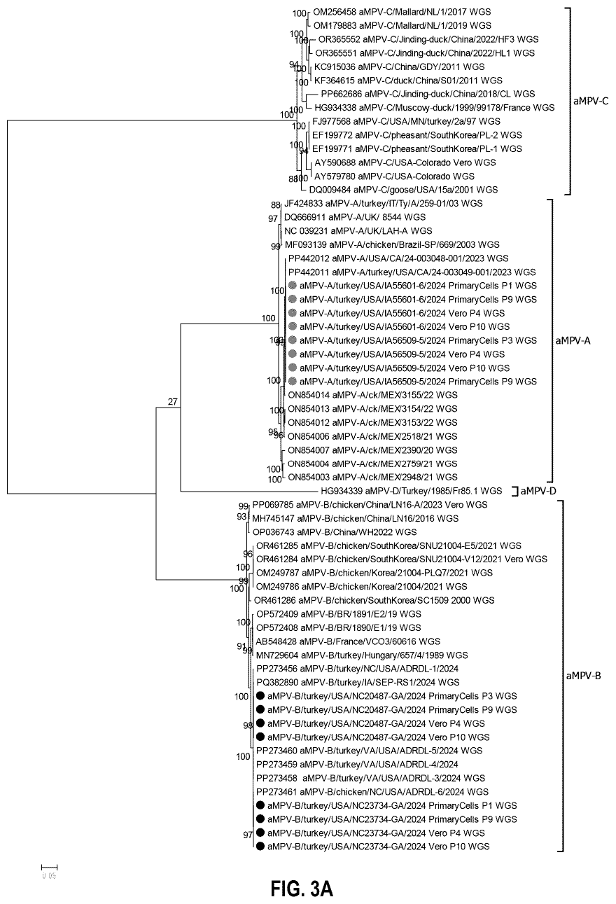

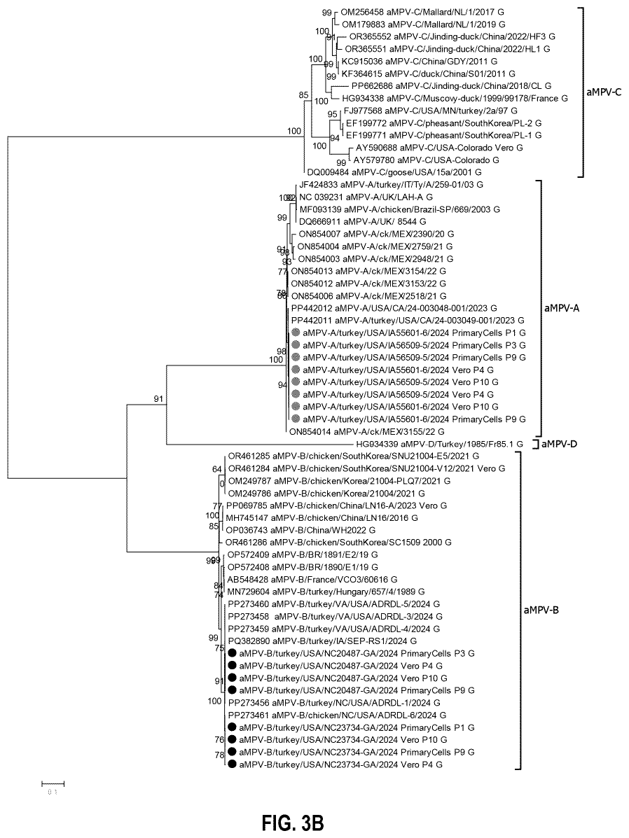

Phylogenetic analysis clearly demonstrated that the aMPV-A and aMPV-B sequences from this study clustered with other aMPV-A and aMPV-B sequences, respectively, regardless of whether the analysis was based on whole genome sequences (FIG. 3A) or G gene nucleotide sequences (FIG. 3B).

### Discussion

Since their emergence in the USA for the first time in late 2023 and early 2024, aMPV-A and aMPV-B have continued to be a growing threat to the U.S. poultry industry. However, the USDA does not officially track aMPV cases and the detailed epidemiology information was lacking. This study analyzed the detection frequency of aMPV-A and aMPV-B in U.S. poultry based on the PCR data on 2,204 clinical samples (1,158 turkey samples, 936 chicken samples, and 110 other breed samples) submitted to the ISU VDL from January to November 2024. Although the data were sourced from a single diagnostic laboratory, the substantial sample size and representation from ≥22 U.S. states ensure that the findings provide valuable insights into the trends of aMPV-A and aMPV-B detection in U.S. poultry. Among the tested samples, a higher percentage of turkey samples (51.04%) than chicken samples (15.6%) were PCR-positive for aMPV-A and/or aMPV-B (TABLE 1). Under the conditions of this study, the overall positive rate for aMPV-A was higher than that for aMPV-B across different age groups of turkeys. A similar trend was observed in chickens, although the difference in detection rates between aMPV-A and aMPV-B was less pronounced than in turkeys (TABLE 1). However, the aMPV-A and aMPV-B positive rates varied by state. For example, in Iowa, the aMPV-A positive rate in turkeys was significantly higher than the aMPV-B positive rate, whereas in North Carolina, the aMPV-B positive rate in turkeys was markedly higher than the aMPV-A positive rate (TABLE 2).

The ongoing spread of aMPV across major poultry-producing regions in the USA emphasizes the urgent need for effective vaccines. To develop inactivated or live attenuated aMPV vaccines through the conventional approaches, it is essential to isolate aMPV-A and aMPV-B that can grow efficiently in cell culture or embryonated chicken/turkey eggs. In previous studies, aMPV VI has been attempted using various approaches. Lu et al. 1994 reported successful isolation of aMPV (subtype unreported) from tracheal samples using tracheal organ culture (TOC) prepared from 19 to 20-day-old chick embryos, and the third TOC harvest was successfully adapted to grow in Vero cells. In another study conducted by Goyal et al. 2000, aMPV-C VI was attempted in chicken embryo fibroblast (CEF) cells, Vero cells, and Quail tumor 35 (QT-35) cells. Four isolates were obtained by inoculation of CEF cells, and one isolate was obtained in QT-35 cells after 3-7 blind passages in cell cultures. Vero cells did not yield any isolate on primary isolation from clinical samples. However, all 5 isolates could be adapted to grow in Vero cells following primary isolation in CEF or QT-35 cells. Kwon et al. 2010 reported that, when 25 clinical samples (12 aMPV-A PCR-positive and 13 aMPV-B PCR-positive) were directly inoculated into Vero cells followed by serial passages, two aMPV-A isolates were obtained. In Mexico, aMPV-A was successfully isolated from clinical samples using primary chicken embryo lung and trachea mixed culture with subsequent adaptation to Vero cells. In China, successful isolation of aMPV-C was achieved by inoculating clinical samples into 11-day-old duck embryos or 8-day-old duck embryos via the yolk sac, followed by further passages in Vero cells.

In this study, aMPV-A and aMPV-B isolation was initially attempted from clinical samples using the Vero cell line, UMNSAH/DF-1 cell line, and embryonated chicken eggs but were unsuccessful. Subsequently, primary CEF and CEL cells were prepared from chicken embryos. Using these primary cells, aMPV-A was successfully isolated from four clinical samples and aMPV-B from three clinical samples. Under the conditions of this study, the aMPV-A VI outcome was slightly better in primary CEL cells compared to primary CEF cells, while the aMPV-B VI outcome was slightly better in primary CEF cells than in primary CEL cells. Notably, it is relatively easier to prepare a large quantity of primary CEF cells compared to primary CEL cells. For instance, a single chicken embryo could yield enough primary CEF cells to populate up to 150 wells of 24-well plates, whereas the primary CEL cells obtained could only populate up to 48 wells. Nonetheless, it is inconvenient to propagate and titrate aMPV or serially passage the virus in primary cells towards virus attenuation for vaccine development. Therefore, two aMPV-A and two aMPV-B isolates obtained in the primary cells were further adapted to grow efficiently in the Vero cell line. The two aMPV-A isolates reached titers of ~10⁴-10⁶ TCID₅₀/ml in Vero cells between P4 and P10, while the two aMPV-B isolates achieved similar titers during P1-P10 in Vero cells. In addition, the two aMPV-A and two aMPV-B isolates were genetically stable in either primary cells or Vero cells up to 10 serial passages.

The availability of the Vero cell-adapted U.S. aMPV-A and aMPV-B isolates provides a valuable tool for studying viral pathogenesis, determining the infectious doses in turkeys and chickens at different ages, evaluating the effectiveness of disinfectants and antivirals, and developing vaccines. Currently, no MLV vaccines based on aMPV-A or aMPV-B strains of the U.S. origin are commercially available. To address this gap, aMPV-A and aMPV-B isolates were successfully serially passaged in Vero cells for over 20 passages. These efforts are ongoing, with the goal of developing live attenuated aMPV-A and aMPV-B vaccines to help mitigate the negative impacts of aMPV infections.

In summary, the detection frequency of aMPV-A and aMPV-B in U.S. poultry was analyzed based on the PCR data. Additionally, multiple aMPV-A and aMPV-B isolates associated with the recent outbreaks in the USA were obtained and characterized. The U.S. aMPV-A strains were found to be genetically closely related, as were the U.S. aMPV-B isolates. These U.S. aMPV-A and aMPV-B cell culture isolates provide valuable tools for further characterization of aMPV properties and for enhancing disease control through development of specific vaccines.

### Example 2: Safety and Efficacy of a Bivalent Inactivated Vaccine Against aMPV Subtypes A and B

This example describes the development of a bivalent inactivated vaccine using the U.S. aMPV-A and aMPV-B isolates and the evaluation of its safety and efficacy against both subtypes in turkeys, compared with monovalent aMPV-A and aMPV-B formulations. Given reports of both aMPV-A and aMPV-B in some states and co-infection of two subtypes on some farms, a bivalent killed vaccine could provide broad protection and reduce the disease impact without requiring two monovalent vaccines.

Preparation of Killed aMPV-A and aMPV-B Vaccine Antigens

Subtypes A and B were successfully isolated in primary cells and adapted to grow efficiently in continuous Vero cell line (Example 1). One U.S. aMPV-A isolate (USA/IA55601-6/2024) and one U.S. aMPV-B isolate (USA/NC20487-GA/2024) have been propagated in Vero cells, yielding 500 ml of each virus at titers of 3.16×10⁶ TCID50/ml for aMPV-A and 7.94×10⁶ TCID50/ml for aMPV-B. Four hundred ml of aMPV-A isolate and 200 ml of aMPV-B isolate will be ultracentrifuged (28,000 rpm for 2 hours) and resuspended into 40 ml, respectively, resulting in 10-fold concentrated aMPV-A and 5-fold concentrated aMPV-B. Theoretically, the concentrated aMPV-A and aMPV-B would have similar titers and this will be confirmed by TCID50 titration in Vero cells.

To inactivate the viruses, the concentrated aMPV-A and aMPV-B isolates will be treated with 0.1 M binary ethyleneimine (BEI) at 3% for a final concentration of 3 mM and incubated at 37° C. for 24 h. Excess BEI will be neutralized with 0.1 mM sodium thiosulfate for 2 h at 37° C. Infectivity of treated virus samples will be tested in Vero cells for three consecutive passages to verify the complete inactivation of the virus.

### Formulating Monovalent and Bivalent Killed Vaccine Antigens

Briefly, a dose of 0.5 ml monovalent antigen will include 0.188 ml virus antigen (killed aMPV-A or aMPV-B), 0.188 ml phosphate buffered saline (PBS), and 0.125 ml specific adjuvant. A dose of 0.5 ml bivalent antigen will include 0.188 ml killed aMPV-A virus antigen, 0.188 ml killed aMPV-B virus antigen, and 0.125 ml specific adjuvant. The negative control inoculum will include 0.376 ml PBS and 0.125 ml specific adjuvant.

### Animal Experimental Design

The safety and efficacy of the inactivated vaccine antigens will be evaluated (TABLE 10). Ninety 2-week-old aMPV naïve turkeys (PCR negative on oropharyngeal swabs and antibody negative on sera) will be transported to the ISU animal facility, and randomly divided into 9 groups with 10 turkeys/group. After 3-day acclimation, turkeys will receive priming vaccination (DO), followed by boosting at D21, with the respective formulated killed virus antigens or negative control inoculum. At D35, turkeys will be challenged (TABLE 10). Clinical signs will be monitored and scored during the study period on a 0-3 scale. Oropharyngeal swabs will be collected at DO, D21, D35, and daily during D36-D42 and tested by quantitative real-time RT-PCR. Serum samples will be collected at DO, D7, D14, D21, D28, D35 and D42 to measure aMPV ELISA antibody and aMPV neutralizing antibody. Fresh and formalin-fixed lung, trachea, and nasal turbinates will be collected for PCR testing and histopathological examinations, respectively. Statistical analyses will be performed.

TABLE 10
Experimental design (90 turkeys)

| | Priming vacc* | Boosting vacc* | Challenge** | Necropsy |
|---|---|---|---|---|
| Group | (D 0) | (D 21) | (D 35) | D 42 |
| G1 AB/A (N = 10) | aMPV-A&B | aMPV-A&B | aMPV-A | All birds |
| G2 AB/B (N = 10) | aMPV-A&B | aMPV-A&B | aMPV-B | All birds |
| G3 A/A (N = 10) | aMPV-A | aMPV-A | aMPV-A | All birds |
| G4 A/B (N = 10) | aMPV-A | aMPV-A | aMPV-B | All birds |
| G5 B/B (N = 10) | aMPV-B | aMPV-B | aMPV-B | All birds |
| G6 B/A (N = 10) | aMPV-B | aMPV-B | aMPV-A | All birds |
| G7 N/A (N = 10) | Neg | Neg | aMPV-A | All birds |
| G8 N/B (N = 10) | Neg | Neg | aMPV-B | All birds |
| G9 N/N (N = 10) | Neg | Neg | Neg | All birds |

*Priming and boosting vaccinations with formulated killed vaccine antigens or formulated negative control inoculum: intramuscular (IM) route, 0.5 ml dose/turkey.
**Challenge: live virus via eye drop and intranasal routes, ~10\hat{ }6 TCID50/turkey.

### REFERENCES

Bayon-Auboyer M H, Arnauld C, Toquin D, Eterradossi N. 2000. Nucleotide sequences of the F, L and G protein genes of two non-A/non-B avian pneumoviruses (APV) reveal a novel APV subgroup. J Gen Virol 81:2723-2733.
Canuti M, Kroyer ANK, Ojkic D, Whitney H G, Robertson G J, Lang A S. 2019. Discovery and Characterization of Novel RNA Viruses in Aquatic North American Wild Birds. Viruses 11.
Chen Q, Wang L, Zheng Y, Zhang J, Guo B, Yoon K J, Gauger P C, Harmon K M, Main R G, Li G. 2018. Metagenomic analysis of the RNA fraction of the fecal virome indicates high diversity in pigs infected by porcine endemic diarrhea virus in the United States. Virol J 15:95.
Cook J K, Jones B V, Ellis M M, Jing L, Cavanagh D. 1993. Antigenic differentiation of strains of turkey rhinotracheitis virus using monoclonal antibodies. Avian Pathol 22:257-73.
Cook J K. 2000. Avian pneumovirus infections of turkeys and chickens. Vet J 160:118-25.
Goraichuk I V, Torchetti M K, Killian M L, Kapczynski D R, Sary K, Kulkarni A, Suarez D L. 2024. Introduction of Avian metapneumovirus subtype A to the United States: molecular insights and implications. Front Microbiol 15:1428248.
Goyal S M, Chiang S J, Dar A M, Nagaraja K V, Shaw D P, Halvorson D A, Kapur V. 2000. Isolation of avian pneumovirus from an outbreak of respiratory illness in Minnesota turkeys. J Vet Diagn Invest 12:166-8.
Guionie O, Toquin D, Sellal E, Bouley S, Zwingelstein F, Allee C, Bougeard S, Lemiere S, Eterradossi N. 2007. Laboratory evaluation of a quantitative real-time reverse transcription PCR assay for the detection and identification of the four subgroups of avian metapneumovirus. J Virol Methods 139:150-8.
Juhasz K, Easton A J. 1994. Extensive sequence variation in the attachment (G) protein gene of avian pneumovirus: evidence for two distinct subgroups. J Gen Virol 75 (Pt 11): 2873-80.
Katoh K, Standley D M. 2013. MAFFT Multiple Sequence Alignment Software Version 7: Improvements in Performance and Usability. Mol Biol Evol 30:772-780.
Kwon J S, Lee H J, Jeong S H, Park J Y, Hong Y H, Lee Y J, Youn H S, Lee D W, Do S H, Park S Y, Choi I S, Lee J B, Song C S. 2010. Isolation and characterization of avian metapneumovirus from chickens in Korea. J Vet Sci 11:59-66.
Lu Y S, Shien Y S, Tsai H J, Tseng C S, Lee S H, Lin D F. 1994. Swollen head syndrome in Taiwan-isolation of an avian pneumovirus and serological survey. Avian Pathol 23:169-74.
Luqman M, Duhan N, Temeeyasen G, Selim M, Jangra S, Mor S K. 2024. Geographical Expansion of Avian Metapneumovirus Subtype B: First Detection and Molecular Characterization of Avian Metapneumovirus Subtype B in US Poultry. Viruses 16.
Nguyen L T, Schmidt H A, von Haeseler A, Minh B Q. 2015. I Q-TREE: a fast and effective stochastic algorithm for estimating maximum-likelihood phylogenies. Mol Biol Evol 32:268-74.
Reed L J, Muench H. 1938. A simple method of estimating fifty percent endpoints. Am J Hyg 27:493-497.
Retallack H, Clubb S, DeRisi J L. 2019. Genome Sequence of a Divergent Avian Metapneumovirus from a Monk Parakeet (Myiopsitta monachus). Microbiol Resour Announc 8.
Rivera-Benitez J F, Martinez-Bautista R, Rios-Cambre F, Ramirez-Mendoza H. 2014. Molecular detection and isolation of avian metapneumovirus in Mexico. Avian Pathol 43:217-23.
Salles GBC, Pilati GVT, Muniz E C, de Lima Neto A J, Vogt J R, Dahmer M, Savi B P, Padilha D A, Fongaro G. 2023. Trends and Challenges in the Surveillance and Control of Avian Metapneumovirus. Viruses 15.
Seal B S. 1998. Matrix protein gene nucleotide and predicted amino acid sequence demonstrate that the first US avian pneumovirus isolate is distinct from European strains. Virus Res 58:45-52.
Sun S, Chen F, Cao S, Liu J, Lei W, Li G, Song Y, Lu J, Liu C, Qin J, Li H. 2014. Isolation and characterization of a subtype C avian metapneumovirus circulating in Muscovy ducks in China. Vet Res 45:74.
Van de Zande S, Nauwynck H, Cavanagh D, Pensaert M. 1998. Infections and reinfections with avian pneumovirus subtype A and B on Belgian turkey farms and relation to respiratory problems. Zentralbl Veterinarmed B 45:621-6.
Velayudhan B T, McComb B, Bennett R S, Lopes V C, Shaw D, Halvorson D A, Nagaraja K V. 2005. Emergence of a virulent type C avian metapneumovirus in turkeys in Minnesota. Avian Dis 49:520-6.
Xu G, Wang B, Qiao Z, Fan L, Jiang S, Zhang R. 2024. Isolation and characterization of an avian metapneumovirus subtype C circulating in Cherry Valley ducks. Poult Sci 104:104650.
Zhang J, Zheng Y, Xia X Q, Chen Q, Bade S A, Yoon K J, Harmon K M, Gauger P C, Main R G, Li G. 2017. High-throughput whole genome sequencing of Porcine reproductive and respiratory syndrome virus from cell culture materials and clinical specimens using next-generation sequencing technology. J Vet Diagn Invest 29:41-50.
Zhu J H, Rawal G, Aljets E, Yim-Im W, Yang Y L, Huang Y W, Krueger K, Gauger P, Main R, Zhang J. 2022. Development and clinical applications of a 5-plex real-time RT-PCR for swine enteric coronaviruses. Viruses 14:1536.

### DEPOSITS

A deposit of the cultures of USA/IA55601-6/2024, USA/IA56509-5/2024, USA/NC20487-GA/2024, and USA/NC23734-GA/2024 is maintained by Iowa State University, having an address at 1800 Christensen Dr, Ames, IA 50011. Access to this deposit will be available during the pendency of the application to the Commissioner of Patents and Trademarks and persons determined by the Commissioner to be entitled thereto upon request. Upon allowance of any claims in the application, the Applicant(s) will make available to the public without restriction a deposit of the cultures with the American Type Culture Collection (ATCC), 10801 University Blvd, Manassas, Virginia, 20110. The cultures deposited with the ATCC will be taken from the same deposit maintained at Iowa State University as described above. Additionally, Applicant(s) will meet all the requirements of 37 C.F.R. § 1.801-1.809, including providing an indication of the viability of the cultures when the deposit is made. This deposit of the aforementioned cultures will be maintained in the ATCC Depository, which is a public depository, for a period of 30 years, or 5 years after the most recent request, or for the enforceable life of the patent, whichever is longer, and will be replaced if it ever becomes nonviable during that period.

What is claimed is:

1. An immunogenic composition comprising an inactivated or live attenuated avian metapneumovirus (aMPV), wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16; and
a pharmaceutically acceptable carrier.

2. The immunogenic composition of claim 1, wherein the aMPV is inactivated, and wherein the composition comprises at least one aMPV subtype A isolate and at least one aMPV subtype B isolate.

3. The immunogenic composition of claim 1, wherein the aMPV is a live attenuated virus.

4. The immunogenic composition of claim 1, wherein the aMPV is attenuated by passaging in cell culture such that when the attenuated virus is administered to an avian subject it fails to cause clinical signs of aMPV but is capable of inducing an immune response that immunizes the avian subject against pathogenic forms of aMPV.

5. The immunogenic composition of claim 1, wherein the pharmaceutically acceptable carrier is a diluent, adjuvant, antimicrobial agent, preservative, inactivating agent, or a combination thereof.

6. A method for inducing an immune response against avian metapneumovirus (aMPV) in an avian subject comprising:
administering to the avian subject an immunogenic composition comprising an inactivated or live attenuated aMPV, wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

7. The method of claim 6, wherein the aMPV is inactivated, and wherein the composition comprises at least one aMPV subtype A isolate and at least one aMPV subtype B isolate.

8. The method of claim 6, wherein the aMPV is a live attenuated virus.

9. The method of claim 6, wherein the aMPV is attenuated by passaging in cell culture such that when the attenuated aMPV is administered to an avian subject it fails to cause clinical signs of aMPV but is capable of inducing an immune response that immunizes the avian subject against pathogenic forms of aMPV.

10. The method of claim 6, wherein the immunogenic composition further comprises a pharmaceutically acceptable carrier, wherein the pharmaceutically acceptable carrier comprises a diluent, adjuvant, antimicrobial agent, preservative, inactivating agent, or a combination thereof.

11. The method of claim 6, wherein the avian subject is a chicken or a turkey.

12. A DNA polynucleotide encoding an avian metapneumovirus (aMPV), or an immunogenic fragment thereof, wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

13. A vector comprising the DNA polynucleotide of claim 12.

14. A host cell comprising the DNA polynucleotide of claim 12.

15. A culture of cells comprising an avian metapneumovirus (aMPV), wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

16. The culture of cells of claim 15, wherein the cells are Vero cells, chicken embryo fibroblast cells, or chicken embryo lung cells.

17. A method of preparing a live attenuated avian metapneumovirus (aMPV) comprising:
passaging an aMPV in cell culture, wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

18. The method of claim 17, wherein the virus is passaged in Vero cells, chicken embryo fibroblast cells, or chicken embryo lung cells.

19. A vaccine composition comprising an inactivated or live attenuated avian metapneumovirus (aMPV), wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16;
an adjuvant; and
a pharmaceutically acceptable carrier.

20. The vaccine composition of claim 19, wherein the aMPV is inactivated, and wherein the composition comprises at least one aMPV subtype A isolate and at least one aMPV subtype B isolate.

21. The vaccine composition of claim 19, wherein the aMPV is a live attenuated virus.

22. The vaccine composition of claim 19, wherein the pharmaceutically acceptable carrier comprises a diluent, antimicrobial agent, preservative, inactivating agent, or a combination thereof.

23. A method of treating or preventing disease caused by avian metapneumovirus (aMPV) comprising:
administering to an avian subject in need thereof a vaccine composition of claim 19.

24. The method of claim 23, wherein the avian subject is a chicken or a turkey.

25. A method for determining if an avian population is in need of vaccination against avian metapneumovirus (aMPV) infection:
collecting a biological sample from one or more subjects of the avian population; and
detecting the presence of the aMPV in the biological sample, wherein the aMPV comprises a polynucleotide having at least 90%, 95%, 96%, 97%, 98%, 99%, 99.5%, or 100% sequence identity to SEQ ID NO: 1, 2, 3, 4, 5, 6, 7, 8, 9, 10, 11, 12, 13, 14, 15, or 16.

26. The method of claim 25, wherein the detecting is by polymerase chain reaction, immunohistochemistry, immunofluorescence, or an indirect fluorescent antibody assay.


SEQUENCE LISTING (22 sequences)

SEQ ID NO: 1 - RNA, 13302 nt, Avian metapneumovirus
  source 1..13302: mol_type = genomic RNA
catttaagcaggttttagaaaaacaatgggacaagtcaaaaatgtctcttgaaagcattagactcagtgacttggagtacaaacatgcaatacttgaagactctcagtatacaattagaagggatgttggtgctaccactgcaattacaccttctgaactgcagccgcaagtatccatattatgtggtatggtgttgtttgcaaaacacaccgactatgagcctgcagcagaggtaggcatgcagtacattagtactgctctaggagctgatagaactcaacaaatactgaaaaattccggtagtgaagtacagggtgttatgaccaagattgtaacacttccagcagagggtcctgtcagaaagcgagaagtgctaaacattcatgatgtaggtgttgggtgggctgatgatgttgaaaggactgcaagagaagcaatgggagcgatggttagagaaaaagtgcaactcacaaagaatcaaaagccgtctgccttggatgctcccgttattctattatgcattggtgccctcattttcaccaagttggcctcaaccgttgaagtaggtcttgaaactgctatccgacgtgcctcaagggtattaagcgatgccatatcacggtaccccaggatggatataccaaggattgccaaatcattctttgaattgtttgagaagaaggtgtattacaggaatttatttattgaatacggtaaggcactcggaggtacatccaccggaagcaggatggagagcctgtttgtgaatatttttatgcaagcttatggggcagggcaaacaatgctgcgctggggtgtcattgcacgatcttccaacaatataatgttaggccatgtatctgtacaggctgagttaaggcaagtatctgaggtctatgacctggtgaggaaaatgggacctgagtcaggattactacacttacgccagagtcccaaagcgggtcttttgtcgttgaccaactgtcccaattttgccagtgttgtcctcgggaacgccgccgggcttggtattataggcatgtacaaaggtcgagcccccaaccttgagctgtttgctgctgctgaaagttatgcacgaacattgaaagagaacaacaagatcaacctagcagccttagggctcactgatgatgagagggaagctgcaacatcctacctagggggagatgatgagagatcatctaagtttgagtaattaaaaaatatgggacaagtaacaatgtctttccctgaaggcaaggatattctaatgatgggcagcgaggctgcaaaaatggccgatgcctatcaaagatcactaaggaacacctctgctggaggccgctctattagtggagaacctattaacacaattgctgaaaaggtgccattaccccctttgtgcaaccccaccacaccaaggggatcatgtataaaacccaacaaggctcctgtctccaaagtcaaagaaatagaatctatatatccaaaactcccaactgctccagttgccactgatactgagacttctaccagtactgagtctgcaaagaagtctaaaaaggtcaagtttgacaaccctaaggtaggaaaatatactaaactggaagaagaggcactagagcttttgtcagatcctgaagaggataatgatgaaaagtcatcaatattgacatttgaggaaaaagatacagcatctacctctattgaagcaagattagaagcaatagaagaaaagttgagcatgatattgggcatgctcaaaacattgaacattgcaactgctggcccaaccgccgccagagatggaatacgtgatgcaatgatagggatgagagaagagctgataaacagcattatgacagaggctaaagacaaaattgcagaaatgatgaaagaagaggatacccagagggcgaaaataggagacggcagcgtgaagcttaccgaaaaagctaaagagttgaacaaagtcctagaagaccaaagctcaagtggagagtcagagagtgaagaagagagtggagagtctgactttaatgaggaagagagtgacatatacaatcttgatctgtagttatgaaaaaatgggacaagtcaaaatggagtcctatataattgacacttatcagggggttccctacactgcagccgttcaggttgacctcattgaaaaagactctaaccctgcaacattgactgtatggtttcccctattccagtcaagtacgcctgccccggtgttattagatcaactaaaaactttatcaatcaccacccagtacacagcatctccggaaggccctgtgctccaagtcaatgcagcagcccaaggtgccgccatgtctgcactacctaagaaatttgccgttagtgctgcagtagcacttgatgagtacagcagactggaattcgggacattgactgtgtgtgatgtgagatccatttatctgactaccttgaagccatatggcatggtctccaaaataatgacagatgtgaggtctgttgggaggaagacccatgacttgatcgcattgtgtgattttatagacatagagaaaggggtgcccataaccatacctgcatatattaaagctgtctctatcaaagactcagaatcagccactgtggaagctgcaataagtggagaagcagaccaggctataactcaagcaaggatagctccatatgcagggctgatcttgattatgactatgaacaaccctaaagggatctttaagaaattgggtgcaggtatgcaagtgatagtagaactggggccgtatgtacaagcagagagtctgggaaaaatctgcaagacatggaatcatcaacgaaccaggtatgtactgaggtcacgttgagggtaccctaaagcataaatctgttaatgtcatagtcaataaaaaataaaaaattgggacaagtaggatggatgtaagaatctgcctcctattgttccttatatccaatccgggtagctgcatacaagaaacatataatgaagaatcttgcagtactgtaactagaggctataagagtgtgttaagaacagggtggtatacgaatgtatttaaccttgaaatagggagtgttgagaacatcacttgcaatgatggacccagcctaattgacactgagttggtactcacaaagaatgctttgagggaactcaaaacagtgtcagctgatcaagtggccaaggaaagcagactatcctcacccaggagacgtagatttgtactgggtgcaattgcacttggcgttgcaacggctgctgccgtaacagctggtgtagcacttgcaaagacaattagattagagggagaggtgaaggcaatcaagaatgccctccggaacacaaatgaggcagtatccacattagggaatggtgtgagggtactagcaactgcagtcaatgacctcaaggaatttataagtaaaaaattgactcctgctattaaccggaacaaatgcaatatagcagatataaagatggcaattagttttggccaaaataacagaaggttcttgaatgtggtgaggcaattctctgatagtgcaggtatcacatcagctgtgtctcttgatttaatgacagatgatgaacttgttagagcaattaacaaaatgccaacttcatcaggacagattagtttgatgttgaacaatcgttcaatggttagaaggaaggggttcggtatattgattggtgtttatgatggaacggtcgtttatatggtacaactgcccatattcggcgtgattgagacaccttgttggagggtggtggcagcaccactctgtagggaagagaaaggcaattatgcttgtatactaagagaagatcaagggtggtactgtacaaatgctggctctacagcttattaccctaatgaagatgattgtgaagtaagggatgactatgtgttttgtgacacagcagctggcattaatgtagcctcagaagttgagcagtgcaactataacatatcgacttctaaatacccatgcaaagtcagcacaggtaggcaccccgtcagtatggtagctttaacccccctagggggtctagtgtcttgttatgagggtgtaagttgctccataggcagcaataaagtagggataataaaacagctaggcaaagggtgcacccacattcccaacaacgaagctgacacaataaccattgataacactgtgtaccaattgagcaaggttgtaggtgaacagaggaccataaaaggagccccagttgtgaacaattttaatccaatattattccctgaggatcagttcaatgttgcacttgaccaagtatttgagagtatagatagatctcaagacttaatagataagtctaacgacttgctaggtgcagatgccaagagcaaggctggaattgctatagcaatagtagtgctagtcattctaggaatcttctttgtgcttgcagtgatatattactgtttcagagtccagaagaccaaaccaaagcatgattatccggccacgacaggtcatagcagcatggcttatgtcagttaagttatttaaaattgggacaagtgaggatgtctaggcgaaatccctgcagatatgaaatacggggcaaatgcaacaggggatcatcctgcacgtttaaccataattattggtcctggcctgatcatgtgctcttagtcagagcaaactacatgcttaaccaattagtgaggaacacagacaggacagatggcctatccttgatatcaggggcaggacgagaagatagaacccaggattttgttctaggatctgccaatgttgtacagaattacatagaagggaacacaacaatcaccaaatcatcagcctgttacagcctgtacaacattataaaacagctccaggaaaatgatgtgaagacatccagagattcaatgcttgaagaccctaaacatgtagcgctacacaacctgatactatcttatgttgatatgagtaaaaatccggcaaatttaatcaacagcctcaagcggcttcctagagaaaaactcaaaaagcttgccaagattatattacagctatctgcagggtcagagagtgacaatgccagtggtaataccctgcaaaagggtgacagcaataattaagtgcaatgcacttggattgtgtatggttcgtaagatatatgattatagtattgcaagctggagtgatttaatagaggaagtagccaatatgatcctaatagatcacattaataggaaacaatgtgtggagtgtcgaaaagattttgagtttatagctatatatacctcatataattagttaattaaaaccaattaagctataagtccaaagaagggtgggacaagtcataatgacttctaccgtcaatcttgggtcagacactgcttcaaaatggaaagtgatcaagtcaaggtgcaattcctgctgcaggatcttggtgagttgtgctgctgtgatttgtgcaatcttagctttgatcttccttgttgctacgatcggattgtctgtgaaactggcattcacagtgcaggagatccacaactgcaagcagaagctatcaggggcgagcacaactacagctgcaatctatactactccaagcacaatgatagaagcactacagacaaatcaacttaaactgacaaccaatgaaggacgtagtactctccctgattgcctggtggagaaaaaattgtgcgaaggtgaggtcaggtatctcaaaactaaaggctgcttgggggcacgagagggtgaagatttgaattgtatagatttagtagtggagtgtgttgggaaaccctgtggtcacaacgaggattacaaagaatgcatatgcaccaacaatgggacagcaactaagtgctgctacatctaatgaatattgacaacactagtgccaaataataggcaacagcattatttaattaaaaagaagaaagatcgggacaagtatctcaatggggtccaaactttatatggttcagggcaccagtgcatatcaaactgcagtggggttctggctaaatattgggaggaggtacatattggctatagtcctatcagctttcgggctgacctgcacagtcactattgcactcactgttagtgtcatagttgaacagtcagtgctagaagagtgcagaaactacaatggaggagatagagattggtggtcaaccacccagaagcagtcaactactgcaccaagtgcgactccaacaggaaattatggaggattacaaacggctcgaacaagaaagtctggaagctgtttgcatgtgcaaatttcttatggtgatatgtataaccgcagtaatactgtactgggtggttttgattgtatgggcttattggctctttgcaaatcaggaccgatttgtcagcgagataatcaagttgacccaacagtcctctgccactgcagggtaggtctttcaagtgtggactgctgcaaggtgaacaagatcagcaccaacagcaacaccacctctgagctccagaagaccaacccgacatggcatagccaagacaacacagactccaatccaaatccccaaagcataactaccagcacagccactctgctctcaacaggtctgggcctcatgctcacatcgaagaccgggacacacaaatcagggccctcccaagccctgccggggagcaacaccaacagaaaaacaaccacggaccgagaactagggtccacaaatcaaccaaattcaaccaataatgtgcaacatgataagcacacccaacgaacgacacccctgccaggctacaacaacacaagaactatcctccagcacacaacactctgggagaagacactcagcacatacaagcccacacactctttaaccaacgaatcagatcaacccctctccacaactcaaaacagcatcaactgtgaacattttaacccccaaggcagagaaaaaatctgctacagagtaggttcttacaactccaatattacaaagcaatgcagaattgatgtgcctttgtgttccacttataacacagtgtgcatgaaaacatactataccgaaccattcaactgttggaggcgtatctggcgctgcttgtgtgatgacggagttggactggttgagtggtgttgcactagttaactaaaaatgaaaacacatacatccgataatagaaaaatacatcagatagtctaaaacaattaaaagaatgaaaacaaggaccaatatggaaatatccgatgagtccgtcgtaaatgtctatctgcctgactcttacctaaagggggtcatttcattcagtgagaccaatgcaattggatcatgtgtactcaaccggccatacattaaagatgaccatacagcacatgttgcaatgactaacccagtgattgaacaccagaggttgagggccctcttcaagagtcttactattagcagagaatatcgagtggttgaacctcttatgatccagaaggagctgctaaaagtcgcagcaggtgcaagactaaagaaactcaagaaatggttgggtagaagcaaggatataagtgaagtcaaattgaaaatggtgactgattggcttaaactgtcacagacaccaggaaggggaaaaataatagattgtattcaagtggagaacttacctgactggctggagcactggtttgattcttggttgatattgaatgacgtcatccaatcttataggtgtttggaagtatcccaaaccagtgctatattacggaaaagtagcctgaacttcttttttgctgtttcttcttttgggtgtataatcataagtcgcaagagtagaagaatatgtttttgcacctacaatcaactcctgacttggaaggaccttgctttaagcagattcaatgcaaatttatgtgtgtgggtgagcaattgcttaaatagcgcacaagacggcctcggactaaggagcaaattagttggtgagttactcaaccgattgtactgtgagactgatgagttattgagcatcactggcaatgaggggtatgggattgtaaaggaatttgaaggattcattatgagtgaaatattgcggatgactgaacatgcacaattcagtgtaaggttccggaatacgctattgaatggcatagctgataaaatagggaagatgagagccgcataccaaagacggacacagaacacatctgttgagcatcatagatacccaagtgagcatgaaatgctggaagcaacagggagagtactgagaataattaagcttctggtgaataatgagattgctaatgctgcagagatgtattttatattccgcatattcggacatcctatggtagaagaaagggaggctatggatgcagtgagggataacagtgaaactgcaaaaatcttaagtctggtggcccttacggagatgaggggggccttcattctaagagtgatcaaaggtttcgtagggaactacaaaagatggccaaagataaagaatccatcaacattgagtcgaaggtggaagatgtacatgaatgcaaaaacatacccaagcaatcttgaattgtgtgtggaagacttcttggagcttgctgggatcagtttttgccaagaattctatgtacctagccagaccagtttggagatggtactgaatgataaagcaatatctccccccaaatctctcatatggtctgtgtatccacgtaactaccttccgcaaaacatacaagacagattcaggttggagtctcttgacagactagagcatgagcgcaccaggagagtgctagagttctacttaaaagatgctaattttaagcaaagtgatctcaaaaaatatgttgtaggtcaagagtacttgaatgacaaagagcatgtggtctccttaacagggaaagaaagggaactaagtgtgggtcgtatgtttgcgatgcaacctggaaagcaaagacaggtgcagatccttgctgagaaattgttatcagataacatagtgccatttttcccagagacattgactaggtatggagatctagaattacaaagaatcatggaactcaagtccgaactatcgtctacaaaaaaacggaaaagtgatagctacaataattatatagcaagagcttcaatagtgactgatttgagtaagtttaaccaagcttttagatatgagactacttcagtgtgtgctgatgttgcagatgaactgcatgggactcaaagccttttctgttggttacacctgactgtatcctccacaaccatgatctgcacttatagacatgctccccctgatacaggtggaatctacgacatagaccagataccggagcaaagcggactgtataggttccacatgggtggcatagaaggctggtgccaaaagatgtggaccatggaagcaatatcacttcttgatgtggtgtctgtgaggaatcgggtgcagctcacatcactgctgaatggggacaaccagtctatagatgtgagtaaacctgtgcgactcacaggtgctcaaactgaaattcaggctgattatagccttgccattaagatgttaactgctgtaagggatgcctactataacataggacacaagttgaaggagggagagacttatgtttcaagagacctccagtttatgagcaagaccatccaatcagaaggcgtaatgtatcctgcttcaattaagaaggttctgcgagtgggtccctggataaacacaatactggatgacatcaaaactagtatggaagctattgggagtctctgtcaagagctagaattccgaggggagtcatttgtgactagtcttataattcggaatttctggttgtacatccagtgctttgtcgaggcaaaaaatcatagcctagcaggagcacaaatatccaatgagcttaatagaactttaaccaaagttaagagattcttcaggttagcagatgagagcagcactgttaacttgttcatgaacataccaatgcagttagggggaggggaccctgttgttgtttataggagtttttatagacggacaccagattttttgacagaggtgttaacacacatggaactactactaacagcaatagagatggataggggagtttatatgaacttcttcttaacactgttgaacacatgcaaaaatgatagagccactcttaccacactgatgagagacccccaagctattgggtcagaaaggcaggctaaggtaacaagcgaaattaatagaacagcagtaactagtgtgctcagccttgcaccaaaccccttgtttagtgatagtgctgtgcatttcagtcagaatgaggaagaaataggcacagttatggaggatatatcacctgtctacccacatggactgagagtggtgtatgaagcattcccattccacaaagctgaaaaagttgtaaacatgattgctggaaccaagtccataacaaatatcctccaaagaacttctgctataagtggtgtagatattgacagagcagtccacatgatgttgctcaacttgggcttgttgggtagaatgcttgagtctggacccatcactgacacaatagatctgaggagcaacaatcggattctttgttgtcagctttccaaaagaataagagaaatgtcatgggatgggttggagattgtcggtgtatcatctccaagcatgttgtctagcttagatattaattatgttacaatagcacagaagccaggtatcattgtagaaaaattctcagctgaaaaaacaacaagagggaaaagaggtccgaaagcaccatgggtaggctcgagcacacaagagaaaaaattggtagctgtttacaacagacaggttttatcgaaagaacaaaaagaccaattagaaaccataggcaaaatcaggtgggtctacaaaggcgttactggcctccgacgtcttctaaacttaatatgcttaggaacactgggtcttccatataagctgttaaaaccacttctacctcggtttatgagcgtcaatttcctacacaggcttgcagtgagcagcaggcctatggagttcccatcatcagtaccggcatataggactacaaactttcattttgatactagtcctataaacaagcggttgagtgagaggtttggcaatgaggacatcaaccttgtgttccaaaatgctataagttgtggaattagtgtcatgtgtgtggttgaacaattgaccgggcgtagccctaaacaagtgatactagaacctattgtagaagatattgacataatgtccgcccctaatttccaaggccgcctgaatttcaagagtgtaagaaagattgtgactgatcaacatatctttaatccggaccacattgacttggttatgcttgggaagttgttattaccaactgtccgtagcaatataaataacaacaaactagcaactgaaaatttctttaatggtaacaacatagtagaggcactaacaagctgtcttgcttgccactggtgcactgttttgattctactcactactgagaatagtattttccagaaagagtggggagatggattcataacagaccatgccttcataaacttcacatggttccttatgagtttcaaaacttatctcctatgccattggcaaactgaaaagaacaagacattggggattgtagaggatcccatagacaggctcatcagagtagacaatagtttttggcggatgatgagcaaggtgttcctggaacccaaagtgaaaagaagacttatgctttacgatacgtctgtattgaatgttctgggtagtatcagtttcaaaaactggtttgttagccggttaagaacagtagactacatggagatcccatggattgtcaatgctgagggtgacatagttgagcaaaaacctgtcaatgaatatctgaaaataatggctgcaggtgtccatgtaaaagtcataatgcttagttactccgatatggcacatgctatgacacgcttgctaaggtgcaagaacatgcaagacaatgtgcccaccataaagaaaacagtatctccaactgatgtcacaccctctgtagaccctactcgggccctactgctgttcccgaaagtcatctttagtaagttgaccacttttaatgcaacatgcagtacagctaagcaatctggcagcaatttgacaaaaaactacatgactttaatgccttggcaccacattaatcgttataactttgtccatagctccacagggtgtaaggtaagtgtaaagggatgtataggcaagttgataacaaaacttgaattaagaattatctacttcatcggtgagggagctgggaatctcatgtccagaactgcatgtgaataccctaatctaaagtttgtatacaggagtctgaaagatgccaatgatcatcatcatcccacagagtatgtgcgtgtaatggggagcatcagcagaataattgacctcggtgaagggttggcaatggaagcaactgatgcaactaagagagaacattgggattttatccacagagtgtgcaaagatcctcttctgctaactatctgtgatgctgaactaaaaaacatggaggatgtcctgcatttagtgatacagtggaggaaacatgtactatcttgtaacatatgcacatcatatgggactaatgtgtacatgtttgttaagtatcatgcgcaatctgaggcaaagaagctaccacattttgtccgagtagtttcaatatatatcatgcaagggagtaaactatcaggatcagaatgttacatgttgttaacattaggacaccagaacatggcaccttgctatggagaggttaggtctgcgaaatcaatcatggctttgacgagcacttttaaacacccctgtagactagacaaatctgccattgaggccaacctgaaatcactagcacccgggttgacactgccacttagtgagcctgctatgcaaaagtacttgttggatctttcaagcttaagtggaattaagaacacgggggcaaccattgggggaagcaaagtagtagagaagaagtgggctttatgcaaagtgtcaaatatagtgaggtggttaagtcacgttctgaaaatgcccaaaggtgatttaaattacgattttttcgaggtgatagaaaacacatacccagacatggtgaagctattggacaacttaaccccgtcagagttgaaaaagctcgtcaaaatcaccgggtacatcctgggcacaaagtaataaggcaatatctactcaacacataattatcacctgaaacacaactgacc

SEQ ID NO: 2 - RNA, 13302 nt, Avian metapneumovirus
  source 1..13302: mol_type = genomic RNA
catttaagcaggttttagaaaaacaatgggacaagtcaaaaatgtctcttgaaagcattagactcagtgacttggagtacaaacatgcaatacttgaagactctcagtatacaattagaagggatgttggtgctaccactgcaattacaccttctgaactgcagccgcaagtatccatattatgtggtatggtgttgtttgcaaaacacaccgactatgagcctgcagcagaggtaggcatgcagtacattagtactgctctaggagctgatagaactcaacaaatactgaaaaattccggtagtgaagtacagggtgttatgaccaagattgtaacacttccagcagagggtcctgtcagaaagcgagaagtgctaaacattcatgatgtaggtgttgggtgggctgatgatgttgaaaggactgcaagagaagcaatgggagcgatggttagagaaaaagtgcaactcacaaagaatcaaaagccgtctgccttggatgctcccgttattctattatgcattggtgccctcattttcaccaagttggcctcaaccgttgaagtaggtcttgaaactgctatccgacgtgcctcaagggtattaagcgatgccatatcacggtaccccaggatggatataccaaggattgccaaatcattctttgaattgtttgagaagaaggtgtattacaggaatttatttattgaatacggtaaggcactcggaggtacatccaccggaagcaggatggagagcctgtttgtgaatatttttatgcaagcttatggggcagggcaaacaatgctgcgctggggtgtcattgcacgatcttccaacaatataatgttaggccatgtatctgtacaggctgagttaaggcaagtatctgaggtctatgacctggtgaggaaaatgggacctgagtcaggattactacacttacgccagagtcccaaggcgggtcttttgtcgttgaccaactgtcccaattttgccagtgttgtcctcgggaacgccgccgggcttggtattataggcatgtacaaaggtcgagcccccaaccttgagctgtttgctgctgctgaaagttatgcacgaacattgaaagagaacaacaagatcaacctagcagccttagggctcactgatgatgagagggaagctgcaacatcctacctagggggagatgatgagagatcatctaagtttgagtaattaaaaaatatgggacaagtaacaatgtctttccctgaaggcaaggatattctaatgatgggcagcgaggctgcaaaaatggccgatgcctatcaaagatcactaaggaacacctctgctggaggccgctctattagtggagaacctattaacacaattgctgaaaaggtgccattaccccctttgtgcaaccccaccacaccaaggggatcatgtataaaacccaacaaggctcctgtctccaaagtcaaagaaatagaatctatatatccaaaactcccaactgctccagttgccactgatactgagacttctaccagtactgagtctgcaaagaagtctaaaaaggtcaagtttgacaaccctaaggtaggaaaatatactaaactggaagaagaggcactagagcttttgtcagatcctgaagaggataatgatgaaaagtcatcaatattgacatttgaggaaaaagatacagcatctacctctattgaagcaagattagaagcaatagaagaaaagttgagcatgatattgggcatgctcaaaacattgaacattgcaactgctggcccaaccgccgccagagatggaatacgtgatgcaatgatagggatgagagaagagctgataaacagcattatgacagaggctaaagacaaaattgcagaaatgatgaaagaagaggatacccagagggcgaaaataggagacggcagcgtgaagcttaccgaaaaagctaaagagttgaacaaagtcctagaagaccaaagctcaagtggagagtcagagagtgaagaagagagtggagagtctgactttaatgaggaagagagtgacatatacaatcttgatctgtagttatgaaaaaatgggacaagtcaaaatggagtcctatataattgacacttatcagggggttccctacactgcagccgttcaggttgacctcattgaaaaagactctaaccctgcaacattgactgtatggtttcccctattccagtcaagtacgcctgccccggtgttattagatcaactaaaaactttatcaatcaccacccagtacacagcatctccggaaggccctgtgctccaagtcaatgcagcagcccaaggtgccgccatgtctgcactacctaagaaatttgccgttagtgctgcagtagcacttgatgagtacagcagactggaattcgggacattgactgtgtgtgatgtgagatccatttatctgactaccttgaagccatatggcatggtctccaaaataatgacagatgtgaggtctgttgggaggaagacccatgacttgatcgcattgtgtgattttatagacatagagaaaggggtgcccataaccatacctgcatatattaaagctgtctctatcaaagactcagaatcagccactgtggaagctgcaataagtggagaagcagaccaggctataactcaagcaaggatagctccatatgcagggctgatcttgattatgactatgaacaaccctaaagggatctttaagaaattgggtgcaggtatgcaagtgatagtagaactggggccgtatgtacaagcagagagtctgggaaaaatctgcaagacatggaatcatcaacgaaccaggtatgtactgaggtcacgttgagggtaccctaaagcataaatctgttaatgtcatagtcaataaaaaataaaaaattgggacaagtaggatggatgtaagaatctgcctcctattgttccttatatccaatccgggtagctgcatacaagaaacatataatgaagaatcttgcagtactgtaactagaggctataagagtgtgttaagaacagggtggtatacgaatgtatttaaccttgaaatagggagtgttgagaacatcacttgcaatgatggacccagcctaattgacactgagttggtactcacaaagaatgctttgagggaactcaaaacagtgtcagctgatcaagtggccaaggaaagcagactatcctcacccaggagacgtagatttgtactgggtgcaattgcacttggcgttgcaacggctgctgccgtaacagctggtgtagcacttgcaaagacaattagattagagggagaggtgaaggcaatcaagaatgccctccggaacacaaatgaggcagtatccacattagggaatggtgtgagggtactagcaactgcagtcaatgacctcaaggaatttataagtaaaaaattgactcctgctattaaccggaacaaatgcaatatagcagatataaagatggcaattagttttggccaaaataacagaaggttcttgaatgtggtgaggcaattctctgatagtgcaggtatcacatcagctgtgtctcttgatttaatgacagatgatgaacttgttagagcaattaacaaaatgccaacttcatcaggacagattagtttgatgttgaacaatcgttcaatggttagaaggaaggggttcggtatattgattggtgtttatgatggaacggtcgtttatatggtacaactgcccatattcggcgtgattgagacaccttgttggagggtggtggcagcaccactctgtagggaagagaaaggcaattatgcttgtatactaagagaagatcaagggtggtactgtacaaatgctggctctacagcttattaccctaatggagatgattgtgaagtaagggatgactatgtgttttgtgacacagcagctggcattaatgtagcctcagaagttgagcagtgcaactataacatatcgacttctaaatacccatgcaaagtcagcacaggtaggcaccccgtcagtatggtagctttaacccccctagggggtctagtgtcttgttatgagggtgtaagttgctccataggcagcaataaagtagggataataaaacagctaggcaaagggtgcacccacattcccaacaacgaagctgacacaataaccattgataacactgtgtaccaattgagcaaggttgtaggtgaacagaggaccataaaaggagccccagttgtgaacaattttaatccaatattattccctgaggatcagttcaatgttgcacttgaccaagtatttgagagtatagatagatctcaagacttaatagataagtctaacgacttgctaggtgcagatgccaagagcaaggctggaattgctatagcaatagtagtgctagtcattctaggaatcttctttgtgcttgcagtgatatattactgtttcagagtccagaagaccaaaccaaagcatgattatccggccacgacaggtcatagcagcatggcttatgtcagttaagttatttaaaattgggacaagtgaggatgtctaggcgaaatccctgcagatatgaaatacggggcaaatgcaacaggggatcatcctgcacgtttaaccataattattggtcctggcctgatcatgtgctcttagtcagagcaaactacatgcttaaccaattagtgaggaacacagacaggacagatggcctatccttgatatcaggggcaggacgagaagatagaacccaggattttgttctaggatctgccaatgttgtacagaattacatagaagggaacacaacaatcaccaaatcatcagcctgttacagcctgtacaacattataaaacagctccaggaaaatgatgtgaagacatccagagattcaatgcttgaagaccctaaacatgtagcgctacacaacctgatactatcttatgttgatatgagtaaaaatccggcaaatttaatcaacagcctcaagcggcttcctagagaaaaactcaaaaagcttgccaagattatattacagctatctgcagggtcagagagtgacaatgccagtggtaataccctgcaaaagggtgacagcaataattaagtgcaatgcacttggattgtgtatggttcgtaagatatatgattatagtattgcaagctggagtgatttaatagaggaagtagccaatatgatcctaatagatcacattaataggaaacaatgtgtggagtgtcgaaaagattttgagtttatagctatatatacctcatataattagttaattaaaaccaattaagctataagtccaaagaagggtgggacaagtcataatgacttctaccgtcaatcttgggtcagacactgcttcaaaatggaaagtgatcaagtcaaggtgcaattcctgctgcaggatcttggtgagttgtgctgctgtgatttgtgcaatcttagctttgatcttccttgttgctacgatcggattgtctgtgaaactggcattcacagtgcaggagatccacaactgcaagcagaagctatcaggggcgagcacaactacagctgcaatctatactactccaagcacaatgatagaagcactacagacaaatcaacttaaactgacaaccaatgaaggacgtagtactctccctgattgcctggtggagaaaaaattgtgcgaaggtgaggtcaggtatctcaaaactaaaggctgcttgggggcacgagagggtgaagatttgaattgtatagatttagtagtggagtgtgttgggaaaccctgtggtcacaacgaggattacaaagaatgcatatgcaccaacaatgggacagcaactaagtgctgctacatctaatgaatattgacaacactagtgccaaataataggcaacagcattatttaattaaaaagaagaaagatcgggacaagtatctcaatggggtccaaactttatatggttcagggcaccagtgcatatcaaactgcagtggggttctggctaaatattgggaggaggtacatattggctatagtcctatcagctttcgggctgacctgcacagtcactattgcactcactgttagtgtcatagttgaacagtcagtgctagaagagtgcagaaactacaatggaggagatagagattggtggtcaaccacccagaagcagtcaactactgcaccaagtgcgactccaacaggaaattatggaggattacaaacggctcgaacaagaaagtctggaagctgtttgcatgtgcaaatttcttatggtgatatgtataaccgcagtaatactgtactgggtggttttgattgtatgggcttattggctctttgcaaatcaggaccgatttgtcagcgagataatcaagttgacccaacagtcctctgccactgcagggtaggtctttcaagtgtggactgctgcaaggtgaacaagatcagcaccaacagcaacaccacctctgagctccagaagaccaacccgacatggcatagccaagacaacacagactccaatccaaatccccaaagcataactaccagcacagccactctgctctcaacaggtctgggcctcatgctcacatcgaagaccgggacacacaaatcagggccctcccaagccctgccggggagcaacaccaacagaaaaacaaccacggaccgagaactagggtccacaaatcaaccaaattcaaccaataatgtgcaacatgataagcacacccaacgaacgacacccctgccaggctacaacaacacaagaactatcctccagcacacaacactctgggagaagacactcagcacatacaagcccacacactctttaaccaacgaatcagatcaacccctctccacaactcaaaacagcatcaactgtgaacattttaacccccaaggcagagaaaaaatctgctacagagtaggttcttacaactccaatattacaaagcaatgcagaattgatgtgcctttgtgttccacttataacacagtgtgcatgaaaacatactataccgaaccattcaactgttggaggcgtatctggcgctgcttgtgtgatgacggagttggactggttgagtggtgttgcactagttaactaaaaatgaaaacacatacatccgataatagaaaaatacatcagatagtctaaaacaattaaaagaatgaaaacaaggaccaatatggaaatatccgatgagtccgtcgtaaatgtctatctgcctgactcttacctaaagggggtcatttcattcagtgagaccaatgcaattggatcatgtgtactcaaccggccatacattaaagatgaccatacagcacatgttgcaatgactaacccagtgattgaacaccagaggttgagggccctcttcaagagtcttactattagcagagaatatcgagtggttgaacctcttatgatccagaaggagctgctaaaagtcgcagcaggtgcaagactaaagaaactcaagaaatggttgggtagaagcaaggatataagtgaagtcaaattgaaaatggtgactgattggcttaaactgtcacagacaccaggaaggggaaaaataatagattgtattcaagtggagaacttacctgactggctggagcactggtttgattcttggttgatattgaatgacgtcatccaatcttataggtgtttggaagtatcccaaaccagtgctatattacggaaaagtagcctgaacttcttttttgctgtttcttcttttgggtgtataatcataagtcgcaagagtagaagaatatgtttttgcacctacaatcaactcctgacttggaaggaccttgctttaagcagattcaatgcaaatttatgtgtgtgggtgagcaattgcttaaatagcgcacaagacggcctcggactaaggagcaaattagttggtgagttactcaaccgattgtactgtgagactgatgagttattgagcatcactggcaatgaggggtatgggattgtaaaggaatttgaaggattcattatgagtgaaatattgcggatgactgaacatgcacaattcagtgtaaggttccggaatacgctattgaatggcatagctgataaaatagggaagatgagagccgcataccaaagacggacacagaacacatctgttgagcatcatagatacccaagtgagcatgaaatgctggaagcaacagggagagtactgagaataattaagcttctggtgaataatgagattgctaatgctgcagagatgtattttatattccgcatattcggacatcctatggtagaagaaagggaggctatggatgcagtgagggataacagtgaaactgcaaaaatcttaagtctggtggcccttacggagatgaggggggccttcattctaagagtgatcaaaggtttcgtagggaactacaaaagatggccaaagataaagaatccatcaacattgagtcgaaggtggaagatgtacatgaatgcaaaaacatacccaagcaatcttgaattgtgtgtggaagacttcttggagcttgctgggatcagtttttgccaagaattctatgtacctagccagaccagtttggagatggtactgaatgataaagcaatatctccccccaaatctctcatatggtctgtgtatccacgtaactaccttccgcaaaacatacaagacagattcaggttggagtctcttgacagactagagcatgagcgcaccaggagagtgctagagttctacttaaaagatgctaattttaagcaaagtgatctcaaaaaatatgttgtaggtcaagagtacttgaatgacaaagagcatgtggtctccttaacagggaaagaaagggaactaagtgtgggtcgtatgtttgcgatgcaacctggaaagcaaagacaggtgcagatccttgctgagaaattgttatcagataacatagtgccatttttcccagagacattgactaggtatggagatctagaattacaaagaatcatggaactcaagtccgaactatcgtctacaaaaaaacggaaaagtgatagctacaataattatatagcaagagcttcaatagtgactgatttgagtaagtttaaccaagcttttagatatgagactacttcagtgtgtgctgatgttgcagatgaactgcatgggactcaaagccttttctgttggttacacctgactgtatcctccacaaccatgatctgcacttatagacatgctccccctgatacaggtggaatctacgacatagaccagataccggagcaaagcggactgtataggttccacatgggtggcatagaaggctggtgccaaaagatgtggaccatggaagcaatatcacttcttgatgtggtgtctgtgaggaatcgggtgcagctcacatcactgctgaatggggacaaccagtctatagatgtgagtaaacctgtgcgactcacaggtgctcaaactgaaattcaggctgattatagccttgccattaagatgttaactgctgtaagggatgcctactataacataggacacaagttgaaggagggagagacttatgtttcaagagacctccagtttatgagcaagaccatccaatcagaaggcgtaatgtatcctgcttcaattaagaaggttctgcgagtgggtccctggataaacacaatactggatgacatcaaaactagtatggaagctattgggagtctctgtcaagagctagaattccgaggggagtcatttgtgactagtcttataattcggaatttctggttgtacatccagtgctttgtcgaggcaaaaaatcatagcctagcaggagcacaaatatccaatgagcttaatagaactttaaccaaagttaagagattcttcaggttagcagatgagagcagcactgttaacttgttcatgaacataccaatgcagttagggggaggggaccctgttgttgtttataggagtttttatagacggacaccagattttttgacagaggtgttaacacacatggaactactactaacagcaatagagatggataggggagtttatatgaacttcttcttaacactgttgaacacatgcaaaaatgatagagccactcttaccacactgatgagagacccccaagctattgggtcagaaaggcaggctaaggtaacaagcgaaattaatagaacagcagtaactagtgtgctcagccttgcaccaaaccccttgtttagtgatagtgctgtgcatttcagtcagaatgaggaagaaataggcacagttatggaggatatatcacctgtctacccacatggactgagagtggtgtatgaagcattcccattccacaaagctgaaaaagttgtaaacatgattgctggaaccaagtccataacaaatatcctccaaagaacttctgctataagtggtgtagatattgacagagcagtccacatgatgttgctcaacttgggcttgttgggtagaatgcttgagtctggacccatcactgacacaatagatctgaggagcaacaatcggattctttgttgtcagctttccaaaagaataagagaaatgtcatgggatgggttggagattgtcggtgtatcatctccaagcatgttgtctagcttagatattaattatgttacaatagcacagaagccaggtatcattgtagaaaaattctcagctgaaaaaacaacaagagggaaaagaggtccgaaagcaccatgggtaggctcgagcacacaagagaaaaaattggtagctgtttacaacagacaggttttatcgaaagaacaaaaagaccaattagaaaccataggcaaaatcaggtgggtctacaaaggcgttactggcctccgacgtcttctaaacttaatatgcttaggaacactgggtcttccatataagctgttaaaaccacttctacctcggtttatgagcgtcaatttcctacacaggcttgcagtgagcagcaggcctatggagttcccatcatcagtaccggcatataggactacaaactttcattttgatactagtcctataaacaggcggttgagtgagaggtttggcaatgaggacatcaaccttgtgttccaaaatgctataagttgtggaattagtgtcatgtgtgtggttgaacaattgaccgggcgtagccctaaacaagtgatactagaacctattgtagaagatattgacataatgtccgcccctaatttccaaggccgcctgaatttcaagagtgtaagaaagattgtgactgatcaacatatctttaatccggaccacattgacttggttatgcttgggaagttgttattaccaactgtccgtagcaatataaataacaacaaactagcaactgaaaatttctttaatggtaacaacatagtagaggcactaacaagctgtcttgcttgccactggtgcactgttttgattctactcactactgagaatagtattttccagaaagagtggggagatggattcataacagaccatgccttcataaacttcacatggttccttatgagtttcaaaacttatctcctatgccattggcaaactgaaaagaacaagacattggggattgtagaggatcccatagacaggctcatcagagtagacaatagtttttggcggatgatgagcaaggtgttcctggaacccaaagtgaaaagaagacttatgctttacgatacgtctgtattgaatgttctgggtagtatcagtttcaaaaactggtttgttagccggttaagaacagtagactacatggagatcccatggattgtcaatgctgagggtgacatagttgagcaaaaacctgtcaatgaatatctgaaaataatggctgcaggtgtccatgtaaaagtcataatgcttagttactccgatatggcacatgctatgacacgcttgctaaggtgcaagaacatgcaagacaatgtgcccaccataaagaaaacagtatctccaactgatgtcacaccctctgtagaccctactcgggccctactgctgttcccgaaagtcatctttagtaagttgaccacttttaatgcaacatgcagtacagctaagcaatctggcagcaatttgacaaaaaactacatgactttaatgccttggcaccacattaatcgttataactttgtccatagctccacagggtgtaaggtaagtgtaaagggatgtataggcaagttgataacaaaacttgaattaagaattatctacttcatcggtgagggagctgggaatctcatgtccagaactgcatgtgaataccctaatctaaagtttgtatacaggagtctgaaagatgccaatgatcatcatcatcccacagagtatgtgcgtgtaatggggagcatcagcagaataattgacctcggtgaagggttggcaatggaagcaactgatgcaactaagagagaacattgggattttatccacagagtgtgcaaagatcctcttctgctaactatctgtgatgctgaactaaaaaacatggaggatgtcctgcatttagtgatacagtggaggaaacatgtactatcttgtaacatatgcacatcatatgggactaatgtgtacatgtttgttaagtatcatgcgcaatctgaggcaaagaagctaccacattttgtccgagtagtttcaatatatatcatgcaagggagtaaactatcaggatcagaatgttacatgttgttaacattaggacaccagaacatggcaccttgctatggagaggttaggtctgcgaaatcaatcatggctttgacgagcacttttaaacacccctgtagactagacaaatctgccattgaggccaacctgaaatcactagcacccgggttgacactgccacttagtgagcctgctatgcaaaagtacttgttggatctttcaagcttaagtggaattaagaacacgggggcaaccattgggggaagcaaagtagtagagaagaagtgggctttatgcaaagtgtcaaatatagtgaggtggttaagtcacgttctgaaaatgcccaaaggtgatttaaattacgattttttcgaggtgatagaaaacacatacccagacatggtgaagctattggacaacttaaccccgtcagagttgaaaaagctcgtcaaaatcaccgggtacatcctgggcacaaagtaataaggcaatatctactcaacacataattatcacctgaaacacaactgacc

SEQ ID NO: 3 - RNA, 13302 nt, Avian metapneumovirus
  source 1..13302: mol_type = genomic RNA
catttaagcaggttttagaaaaacaatgggacaagtcaaaaatgtctcttgaaagcattagactcagtgacttggagtacaaacatgcaatacttgaagactctcagtatacaattagaagggatgttggtgctaccactgcaattacaccttctgaactgcagccgcaagtatccatattatgtggtatggtgttgtttgcaaaacacaccgactatgagcctgcagcagaggtaggcatgcagtacattagtactgctctaggagctgatagaactcaacaaatactgaaaaattccggtagtgaagtacagggtgttatgaccaagattgtaacacttccagcagagggtcctgtcagaaagcgagaagtgctaaacattcatgatgtaggtgttgggtgggctgatgatgttgaaaggactgcaagagaagcaatgggagcgatggttagagaaaaagtgcaactcacaaagaatcaaaagccgtctgccttggatgctcccgttattctattatgcattggtgccctcattttcaccaagttggcctcaaccgttgaagtaggtcttgaaactgctatccgacgtgcctcaagggtattaagcgatgccatatcacggtaccccaggatggatataccaaggattgccaaatcattctttgaattgtttgagaagaaggtgtattacaggaatttatttattgaatacggtaaggcactcggaggtacatccaccggaagcaggatggagagcctgtttgtgaatatttttatgcaagcttatggggcagggcaaacaatgctgcgctggggtgtcattgcacgatcttccaacaatataatgttaggccatgtatctgtacaggctgagttaaggcaagtatctgaggtctatgacctggtgaggaaaatgggacctgagtcaggattactacacttacgccagagtcccaaggcgggtcttttgtcgttgaccaactgtcccaattttgccagtgttgtcctcgggaacgccgccgggcttggtattataggcatgtacaaaggtcgagcccccaaccttgagctgtttgctgctgctgaaagttatgcacgaacattgaaagagaacaacaagatcaacctagcagccttagggctcactgatgatgagagggaagctgcaacatcctacctagggggagatgatgagagatcatctaagtttgagtaattaaaaaatatgggacaagtaacaatgtctttccctgaaggcaaggatattctaatgatgggcagcgaggctgcaaaaatggccgatgcctatcaaagatcactaaggaacacctctgctggaggccgctctattagtggagaacctattaacacaattgctgaaaaggtgccattaccctctttgtgcaaccccaccacaccaaggggatcatgtataaaacccaacaaggctcctgtctccaaagtcaaagaaatagaatctatatatccaaaactcccaactgctccagttgccactgatactgagacttctaccagtactgagtctgcaaagaagtctaaaaaggtcaagtttgacaaccctaaggtaggaaaatatactaaactggaagaagaggcactagagcttttgtcagatcctgaagaggataatgatgaaaagtcatcaatattgacatttgaggaaaaagatacagcatctacctctattgaagcaagattagaagcaatagaagaaaagttgagcatgatattgggcatgctcaaaacattgaacattgcaactgctggcccaaccgccgccagagatggaatacgtgatgcaatgatagggatgagagaagagctgataaacagcattatgacagaggctaaagacaaaattgcagaaatgatgaaagaagaggatacccagagggcgaaaataggagacggcagcgtgaagcttaccgaaaaagctaaagagttgaacaaagtcctagaagaccaaagctcaagtggagagtcagagagtgaagaagagagtggagagtctgactttaatgaggaagagagtgacatatacaatcttgatctgtagttatgaaaaaatgggacaagtcaaaatggagtcctatataattgacacttatcagggggttccctacactgcagccgttcaggttgacctcattgaaaaagactctaaccctgcaacattgactgtatggtttcccctattccagtcaagtacgcctgccccggtgttattagatcaactaaaaactttatcaatcaccacccagtacacagcatctccggaaggccctgtgctccaagtcaatgcagcagcccaaggtgccgccatgtctgcactacctaagaaatttgccgttagtgctgcagtagcacttgatgagtacagcagactggaattcgggacattgactgtgtgtgatgtgagatccatttatctgactaccttgaagccatatggcatggtctccaaaataatgacagatgtgaggtctgttgggaggaagacccatgacttgatcgcattgtgtgattttatagacatagagaaaggggtgcccataaccatacctgcatatattaaagctgtctctatcaaagactcagaatcagccactgtggaagctgcaataagtggagaagcagaccaggctataactcaagcaaggatagctccatatgcagggctgatcttgattatgactatgaacaaccctaaagggatctttaagaaattgggtgcaggtatgcaagtgatagtagaactggggccgtatgtacaagcagagagtctgggaaaaatctgcaagacatggaatcatcaacgaaccaggtatgtactgaggtcacgttgagggtaccctaaagcataaatctgttaatgtcatagtcaataaaaaataaaaaattgggacaagtaggatggatgtaagaatctgcctcctattgttccttatatccaatccgggtagctgcatacaagaaacatataatgaagaatcttgcagtactgtaactagaggctataagagtgtgttaagaacagggtggtatacgaatgtatttaaccttgaaatagggagtgttgagaacatcacttgcaatgatggacccagcctaattgacactgagttggtactcacaaagaatgctttgagggaactcaaaacagtgtcagctgatcaagtggccaaggaaagcagactatcctcacccaggagacgtagatttgtactgggtgcaattgcacttggcgttgcaacggctgctgccgtaacagctggtgtagcacttgcaaagacaattagattagagggagaggtgaaggcaatcaagaatgccctccggaacacaaatgaggcagtatccacattagggaatggtgtgagggtactagcaactgcagtcaatgacctcaaggaatttataagtaaaaaattgactcctgctattaaccggaacaaatgcaatatagcagatataaagatggcaattagttttggccaaaataacagaaggttcttgaatgtggtgaggcaattctctgatagtgcaggtatcacatcagctgtgtctcttgatttaatgacagatgatgaacttgttagagcaattaacaaaatgccaacttcatcaggacagattagtttgatgttgaacaatcgttcaatggttagaaggaaggggttcggtatattgattggtgtttatgatggaacggtcgtttatatggtacaactgcccatattcggcgtgattgagacaccttgttggagggtggtggcagcaccactctgtagggaagagaaaggcaattatgcttgtatactaagagaagatcaagggtggtactgtacaaatgctggctctacagcttattaccctaatggagatgattgtgaagtaagggatgactatgtgttttgtgacacagcagctggcattaatgtagcctcagaagttgagcagtgcaactataacatatcgacttctaaatacccatgcaaagtcagcacaggtaggcaccccgtcagtatggtagctttaacccccctagggggtctagtgtcttgttatgagggtgtaagttgctccataggcagcaataaagtagggataataaaacagctaggcaaagggtgcacccacattcccaacaacgaagctgacacaataaccattgataacactgtgtaccaattgagcaaggttgtaggtgaacagaggaccataaaaggagccccagttgtgaacaattttaatccaatattattccctgaggatcagttcaatgttgcacttgaccaagtatttgagagtatagatagatctcaagacttaatagataagtctaacgacttgctaggtgcagatgccaagagcaaggctggaattgctatagcaatagtagtgctagtcattctaggaatcttctttgtgcttgcagtgatatattactgtttcagagtccagaagaccaaaccaaagcatgattatccggccacgacaggtcatagcagcatggcttatgtcagttaagttatttaaaattgggacaagtgaggatgtctaggcgaaatccctgcagatatgaaatacggggcaaatgcaacaggggatcatcctgcacgtttaaccataattattggtcctggcctgatcatgtgctcttagtcagagcaaactacatgcttaaccaattagtgaggaacacagacaggacagatggcctatccttgatatcaggggcaggacgagaagatagaacccaggattttgttctaggatctgccaatgttgtacagaattacatagaagggaacacaacaatcaccaaatcatcagcctgttacagcctgtacaacattataaaacagctccaggaaaatgatgtgaagacatccagagattcaatgcttgaagaccctaaacatgtagcgctacacaacctgatactatcttatgttgatatgagtaaaaatccggcaaatttaatcaacagcctcaagcggcttcctagagaaaaactcaaaaagcttgccaagattatattacagctatctgcagggtcagagagtgacaatgccagtggtaataccctgcaaaagggtgacagcaataattaagtgcaatgcacttggattgtgtatggttcgtaagatatatgattatagtattgcaagctggagtgatttaatagaggaagtagccaatatgatcctaatagatcacattaataggaaacaatgtgtggagtgtcgaaaagattttgagtttatagctatatatacctcatataattagttaattaaaaccaattaagctataagtccaaagaagggtgggacaagtcataatgacttctaccgtcaatcttgggtcagacactgcttcaaaatggaaagtgatcaagtcaaggtgcaattcctgctgcaggatcttggtgagttgtgctgctgtgatttgtgcaatcttagctttgatcttccttgttgctacgatcggattgtctgtgaaactggcattcacagtgcaggagatccacaactgcaagcagaagctatcaggggcgagcacaactacagctgcaatctatactactccaagcacaatgatagaagcactacagacaaatcaacttaaactgacaaccaatgaaggacgtagtactctccctgattgcctggtggagaaaaaattgtgcgaaggtgaggtcaggtatctcaaaactaaaggctgcttgggggcacgagagggtgaagatttgaattgtatagatttagtagtggagtgtgttgggaaaccctgtggtcacaacgaggattacaaagaatgcatatgcaccaacaatgggacagcaactaagtgctgctacatctaatgaatattgacaacactagtgccaaataataggcaacagcattatttaattaaaaagaagaaagatcgggacaagtatctcaatggggtccaaactttatatggttcagggcaccagtgcatatcaaactgcagtggggttctggctaaatattgggaggaggtacatattggctatagtcctatcagctttcgggctgacctgcacagtcactattgcactcactgttagtgtcatagttgaacagtcagtgctagaagagtgcagaaactacaatggaggagatagagattggtggtcaaccacccagaagcagtcaactactgcaccaagtgcgactccaacaggaaattatggaggattacaaacggctcgaacaagaaagtctggaagctgtttgcatgtgcaaatttcttatggtgatatgtataaccgcagtaatactgtactgggtggttttgattgtatgggcttattggctctttgcaaatcaggaccgatttgtcagcgagataatcaagttgacccaacagtcctctgccactgcagggtaggtctttcaagtgtggactgctgcaaggtgaacaagatcagcaccaacagcaacaccacctctgagctccagaagaccaacccgacatggcatagccaagacaacacagactccaatccaaatccccaaagcataactaccagcacagccactctgctctcaacaggtctgggcctcatgctcacatcgaagaccgggacacacaaatcagggccctcccaagccctgccggggagcaacaccaacagaaaaacaaccacggaccgagaactagggtccacaaatcaaccaaattcaaccaataatgtgcaacatgataagcacacccaacgaacgacacccctgccaggctacaacaacacaagaactatcctccagcacacaacactctgggagaagacactcagcacatacaagcccacacactctttaaccaacgaatcagatcaacccctctccacaactcaaaacagcatcaactgtgaacattttaacccccaaggcagagaaaaaatctgctacagagtaggttcttacaactccaatattacaaagcaatgcagaattgatgtgcctttgtgttccacttataacacagtgtgcatgaaaacatactataccgaaccattcaactgttggaggcgtatctggcgctgcttgtgtgatgacggagttggactggttgagtggtgttgcactagttaactaaaaatgaaaacacatacatccgataatagaaaaatacatcagatagtctaaaacaattaaaagaatgaaaacaaggaccaatatggaaatatccgatgagtccgtcgtaaatgtctatctgcctgactcttacctaaagggggtcatttcattcagtgagaccaatgcaattggatcatgtgtactcaaccggccatacattaaagatgaccatacagcacatgttgcaatgactaacccagtgattgaacaccagaggttgagggccctcttcaagagtcttactattagcagagaatatcgagtggttgaacctcttatgatccagaaggagctgctaaaagtcgcagcaggtgcaagactaaagaaactcaagaaatggttgggtagaagcaaggatataagtgaagtcaaattgaaaatggtgactgattggcttaaactgtcacagacaccaggaaggggaaaaataatagattgtattcaagtggagaacttacctgactggctggagcactggtttgattcttggttgatattgaatgacgtcatccaatcttataggtgtttggaagtatcccaaaccagtgctatattacggaaaagtagcctgaacttcttttttgctgtttcttcttttgggtgtataatcataagtcgcaagagtagaagaatatgtttttgcacctacaatcaactcctgacttggaaggaccttgctttaagcagattcaatgcaaatttatgtgtgtgggtgagcaattgcttaaatagcgcacaagacggcctcggactaaggagcaaattagttggtgagttactcaaccgattgtactgtgagactgatgagttattgagcatcactggcaatgaggggtatgggattgtaaaggaatttgaaggattcattatgagtgaaatattgcggatgactgaacatgcacaattcagtgtaaggttccggaatacgctattgaatggcatagctgataaaatagggaagatgagagccgcataccaaagacggacacagaacacatctgttgagcatcatagatacccaagtgagcatgaaatgctggaagcaacagggagagtactgagaataattaagcttctggtgaataatgagattgctaatgctgcagagatgtattttatattccgcatattcggacatcctatggtagaagaaagggaggctatggatgcagtgagggataacagtgaaactgcaaaaatcttaagtctggtggcccttacggagatgaggggggccttcattctaagagtgatcaaaggtttcgtagggaactacaaaagatggccaaagataaagaatccatcaacattgagtcgaaggtggaagatgtacatgaatgcaaaaacatacccaagcaatcttgaattgtgtgtggaagacttcttggagcttgctgggatcagtttttgccaagaattctatgtacctagccagaccagtttggagatggtactgaatgataaagcaatatctccccccaaatctctcatatggtctgtgtatccacgtaactaccttccgcaaaacatacaagacagattcaggttggagtctcttgacagactagagcatgagcgcaccaggagagtgctagagttctacttaaaagatgctaattttaagcaaagtgatctcaaaaaatatgttgtaggtcaagagtacttgaatgacaaagagcatgtggtctccttaacagggaaagaaagggaactaagtgtgggtcgtatgtttgcgatgcaacctggaaagcaaagacaggtgcagatccttgctgagaaattgttatcagataacatagtgccatttttcccagagacattgactaggtatggagatctagaattacaaagaatcatggaactcaagtccgaactatcgtctacaaaaaaacggaaaagtgatagctacaataattatatagcaagagcttcaatagtgactgatttgagtaagtttaaccaagcttttagatatgagactacttcagtgtgtgctgatgttgcagatgaactgcatgggactcaaagccttttctgttggttacacctgactgtatcctccacaaccatgatctgcacttatagacatgctccccctgatacaggtggaatctacgacatagaccagataccggagcaaagcggactgtataggttccacatgggtggcatagaaggctggtgccaaaagatgtggaccatggaagcaatatcacttcttgatgtggtgtctgtgaggaatcgggtgcagctcacatcactgctgaatggggacaaccagtctatagatgtgagtaaacctgtgcgactcacaggtgctcaaactgaaattcaggctgattatagccttgccattaagatgttaactgctgtaagggatgcctactataacataggacacaagttgaaggagggagagacttatgtttcaagagacctccagtttatgagcaagaccatccaatcagaaggcgtaatgtatcctgcttcaattaagaaggttctgcgagtgggtccctggataaacacaatactggatgacatcaaaactagtatggaagctattgggagtctctgtcaagagctagaattccgaggggagtcatttgtgactagtcttataattcggaatttctggttgtacatccagtgctttgtcgaggcaaaaaatcatagcctagcaggagcacaaatatccaatgagcttaatagaactttaaccaaagttaagagattcttcaggttagcagatgagagcagcactgttaacttgttcatgaacataccaatgcagttagggggaggggaccctgttgttgtttataggagtttttatagacggacaccagattttttgacagaggtgttaacacacatggaactactactaacagcaatagagatggataggggagtttatatgaacttcttcttaacactgttgaacacatgcaaaaatgatagagccactcttaccacactgatgagagacccccaagctattgggtcagaaaggcaggctaaggtaacaagcgaaattaatagaacagcagtaactagtgtgctcagccttgcaccaaaccccttgtttagtgatagtgctgtgcatttcagtcagaatgaggaagaaataggcacagttatggaggatatatcacctgtctacccacatggactgagagtggtgtatgaagcattcccattccacaaagctgaaaaagttgtaaacatgattgctggaaccaagtccataacaaatatcctccaaagaacttctgctataagtggtgtagatattgacagagcagtccacatgatgttgctcaacttgggcttgttgggtagaatgcttgagtctggacccatcactgacacaatagatctgaggagcaacaatcggattctttgttgtcagctttccaaaagaataagagaaatgtcatgggatgggttggagattgtcggtgtatcatctccaagcatgttgtctagcttagatattaattatgttacaatagcacagaagccaggtatcattgtagaaaaattctcagctgaaaaaacaacaagagggaaaagaggtccgaaagcaccatgggtaggctcgagcacacaagagaaaaaattggtagctgtttacaacagacaggttttatcgaaagaacaaaaagaccaattagaaaccataggcaaaatcaggtgggtctacaaaggcgttactggcctccgacgtcttctaaacttaatatgcttaggaacactgggtcttccatataagctgttaaaaccacttctacctcggtttatgagcgtcaatttcctacacaggcttgcagtgagcagcaggcctatggagttcccatcatcagtaccggcatataggactacaaactttcattttgatactagtcctataaacaggcggttgagtgagaggtttggcaatgaggacatcaaccttgtgttccaaaatgctataagttgtggaattagtgtcatgtgtgtggttgaacaattgaccgggcgtagccctaaacaagtgatactagaacctattgtagaagatattgacataatgtccgcccctaatttccaaggccgcctgaatttcaagagtgtaagaaagattgtgactgatcaacatatctttaatccggaccacattgacttggttatgcttgggaagttgttattaccaactgtccgtagcaatataaataacaacaaactagcaactgaaaatttctttaatggtaacaacatagtagaggcactaacaagctgtcttgcttgccactggtgcactgttttgattctactcactactgagaatagtattttccagaaagagtggggagatggattcataacagaccatgccttcataaacttcacatggttccttatgagtttcaaaacttatctcctatgccattggcaaactgaaaagaacaagacattggggattgtagaggatcccatagacaggctcatcagagtagacaatagtttttggcggatgatgagcaaggtgttcctggaacccaaagtgaaaagaagacttatgctttacgatacgtctgtattgaatgttctgggtagtatcagtttcaaaaactggtttgttagccggttaagaacagtagactacatggagatcccatggattgtcaatgctgagggtgacatagttgagcaaaaacctgtcaatgaatatctgaaaataatggctgcaggtgtccatgtaaaagtcataatgcttagttactccgatatggcacatgctatgacacgcttgctaaggtgcaagaacatgcaagacaatgtgcccaccataaagaaaacagtatctccaactgatgtcacaccctctgtagaccctactcgggccctactgctgttcccgaaagtcatctttagtaagttgaccacttttaatgcaacatgcagtacagctaagcaatctggcagcaatttgacaaaaaactacatgactttaatgccttggcaccacattaatcgttataactttgtccatagctccacagggtgtaaggtaagtgtaaagggatgtataggcaagttgataacaaaacttgaattaagaattatctacttcatcggtgagggagctgggaatctcatgtccagaactgcatgtgaataccctaatctaaagtttgtatacaggagtctgaaagatgccaatgatcatcatcatcccacagagtatgtgcgtgtaatggggagcatcagcagaataattgacctcggtgaagggttggcaatggaagcaactgatgcaactaagagagaacattgggattttatccacagagtgtgcaaagatcctcttctgctaactatctgtgatgctgaactaaaaaacatggaggatgtcctgcatttagtgatacagtggaggaaacatgtactatcttgtaacatatgcacatcatatgggactaatgtgtacatgtttgttaagtatcatgcgcaatctgaggcaaagaagctaccacattttgtccgagtagtttcaatatatatcatgcaagggagtaaactatcaggatcagaatgttacatgttgttaacattaggacaccagaacatggcaccttgctatggagaggttaggtctgcgaaatcaatcatggctttgacgagcacttttaaacacccctgtagactagacaaatctgccattgaggccaacctgaaatcactagcacccgggttgacactgccacttagtgagcctgctatgcaaaagtacttgttggatctttcaagcttaagtggaattaagaacacgggggcaaccattgggggaagcaaagtagtagagaagaagtgggctttatgcaaagtgtcaaatatagtgaggtggttaagtcacgttctgaaaatgcccaaaggtgatttaaattacgattttttcgaggtgatagaaaacacatacccagacatggtgaagctattggacaacttaaccccgtcagagttgaaaaagctcgtcaaaatcaccgggtacatcctgggcacaaagtaataaggcaatatctactcaacacataattatcacctgaaacacaactgacc

SEQ ID NO: 4 - RNA, 13302 nt, Avian metapneumovirus
  source 1..13302: mol_type = genomic RNA
catttaagcaggttttagaaaaacaatgggacaagtcaaaaatgtctcttgaaagcattagactcagtgacttggagtacaaacatgcaatacttgaagactctcagtatacaattagaagggatgttggtgctaccactgcaattacaccttctgaactgcagccgcaagtatccatattatgtggtatggtgttgtttgcaaaacacaccgactatgagcctgcagcagaggtaggcatgcagtacattagtactgctctaggagctgatagaactcaacaaatactgaaaaattccggtagtgaagtacagggtgttatgaccaagattgtaacacttccagcagagggtcctgtcagaaagcgagaagtgctaaacattcatgatgtaggtgttgggtgggctgatgatgttgaaaggactgcaagagaagcaatgggagcgatggttagagaaaaagtgcaactcacaaagaatcaaaagccgtctgccttggatgctcccgttattctattatgcattggtgccctcattttcaccaagttggcctcaaccgttgaagtaggtcttgaaactgctatccgacgtgcctcaagggtattaagcgatgccatatcacggtaccccaggatggatataccaaggattgccaaatcattctttgaattgtttgagaagaaggtgtattacaggaatttatttattgaatacggtaaggcactcggaggtacatccaccggaagcaggatggagagcctgtttgtgaatatttttatgcaagcttatggggcagggcaaacaatgctgcgctggggtgtcattgcacgatcttccaacaatataatgttaggccatgtatctgtacaggctgagttaaggcaagtatctgaggtctatgacctggtgaggaaaatgggacctgagtcaggattactacacttacgccagagtcccaaagcgggtcttttgtcgttgaccaactgtcccaattttgccagtgttgtcctcgggaacgccgccgggcttggtattataggcatgtacaaaggtcgagcccccaaccttgagctgtttgctgctgctgaaagttatgcacgaacattgaaagagaacaacaagatcaacctagcagccttagggctcactgatgatgagagggaagctgcaacatcctacctagggggagatgatgagagatcatctaagtttgagtaattaaaaaatatgggacaagtaacaatgtctttccctgaaggcaaggatattctaatgatgggcagcgaggctgcaaaaatggccgatgcctatcaaagatcactaaggaacacctctgctggaggccgctctattagtggagaacctattaacacaattgctgaaaaggtgccattaccccctttgtgcaaccccaccacaccaaggggatcatgtataaaacccaacaaggctcctgtctccaaagtcaaagaaatagaatctatatatccaaaactcccaactgctccagttgccactgatactgagacttctaccagtactgagtctgcaaagaagtctaaaaaggtcaagtttgacaaccctaaggtaggaaaatatactaaactggaagaagaggcactagagcttttgtcagatcctgaagaggataatgatgaaaagtcatcaatattgacatttgaggaaaaagatacagcatctacctctattgaagcaagattagaagcaatagaagaaaagttgagcatgatattgggcatgctcaaaacattgaacattgcaactgctggcccaaccgccgccagagatggaatacgtgatgcaatgatagggatgagagaagagctgataaacagcattatgacagaggctaaagacaaaattgcagaaatgatgaaagaagaggatacccagagggcgaaaataggagacggcagcgtgaagcttaccgaaaaagctaaagagttgaacaaagtcctagaagaccaaagctcaagtggagagtcagagagtgaagaagagagtggagagtctgactttaatgaggaagagagtgacatatacaatcttgatctgtagttatgaaaaaatgggacaagtcaaaatggagtcctatataattgacacttatcagggggttccctacactgcagccgttcaggttgacctcattgaaaaagactctaaccctgcaacattgactgtatggtttcccctattccagtcaagtacgcctgccccggtgttattagatcaactaaaaactttatcaatcaccacccagtacacagcatctccggaaggccctgtgctccaagtcaatgcagcagcccaaggtgccgccatgtctgcactacctaagaaatttgccgttagtgctgcagtagcacttgatgagtacagcagactggaattcgggacattgactgtgtgtgatgtgagatccatttatctgactaccttgaagccatatggcatggtctccaaaataatgacagatgtgaggtctgttgggaggaagacccatgacttgatcgcattgtgtgattttatagacatagagaaaggggtgcccataaccatacctgcatatattaaagctgtctctatcaaagactcagaatcagccactgtggaagctgcaataagtggagaagcagaccaggctataactcaagcaaggatagctccatatgcagggctgatcttgattatgactatgaacaaccctaaagggatctttaagaaattgggtgcaggtatgcaagtgatagtagaactggggccgtatgtacaagcagagagtctgggaaaaatctgcaagacatggaatcatcaacgaaccaggtatgtactgaggtcacgttgagggtaccctaaagcataaatctgttaatgtcatagtcaataaaaaataaaaaattgggacaagtaggatggatgtaagaatctgcctcctattgttccttatatccaatccgggtagctgcatacaagaaacatataatgaagaatcttgcagtactgtaactagaggctataagagtgtgttaagaacagggtggtatacgaatgtatttaaccttgaaatagggagtgttgagaacatcacttgcaatgatggacccagcctaattgacactgagttggtactcacaaagaatgctttgagggaactcaaaacagtgtcagctgatcaagtggccaaggaaagcagactatcctcacccaggagacgtagatttgtactgggtgcaattgcacttggcgttgcaacggctgctgccgtaacagctggtgtagcacttgcaaagacaattagattagagggagaggtgaaggcaatcaagaatgccctccggaacacaaatgaggcagtatccacattagggaatggtgtgagggtactagcaactgcagtcaatgacctcaaggaatttataagtaaaaaattgactcctgctattaaccggaacaaatgcaatatagcagatataaagatggcaattagttttggccaaaataacagaaggttcttgaatgtggtgaggcaattctctgatagtgcaggtatcacatcagctgtgtctcttgatttaatgacagatgatgaacttgttagagcaattaacaaaatgccaacttcatcaggacagattagtttgatgttgaacaatcgttcaatggttagaaggaaggggttcggtatattgattggtgtttatgatggaacggtcgtttatatggtacaactgcccatattcggcgtgattgagacaccttgttggagggtggtggcagcaccactctgtagggaagagaaaggcaattatgcttgtatactaagagaagatcaagggtggtactgtacaaatgctggctctacagcttattaccctaatggagatgattgtgaagtaagggatgactatgtgttttgtgacacagcagctggcattaatgtagcctcagaagttgagcagtgcaactataacatatcgacttctaaatacccatgcaaagtcagcacaggtaggcaccccgtcagtatggtagctttaacccccctagggggtctagtgtcttgttatgagggtgtaagttgctccataggcagcaataaagtagggataataaaacagctaggcaaagggtgcacccacattcccaacaacgaagctgacacaataaccattgataacactgtgtaccaattgagcaaggttgtaggtgaacagaggaccataaaaggagccccagttgtgaacaattttaatccaatattattccctgaggatcagttcaatgttgcacttgaccaagtatttgagagtatagatagatctcaagacttaatagataagtctaacgacttgctaggtgcagatgccaagagcaaggctggaattgctatagcaatagtagtgctagtcattctaggaatcttctttgtgcttgcagtgatatattactgtttcagagtccagaagaccaaaccaaagcatgattatccggccacgacaggtcatagcagcatggcttatgtcagttaagttatttaaaattgggacaagtgaggatgtctaggcgaaatccctgcagatatgaaatacggggcaaatgcaacaggggatcatcctgcacgtttaaccataattattggtcctggcctgatcatgtgctcttagtcagagcaaactacatgcttaaccaattagtgaggaacacagacaggacagatggcctatccttgatatcaggggcaggacgagaagatagaacccaggattttgttctaggatctgccaatgttgtacagaattacatagaagggaacacaacaatcaccaaatcatcagcctgttacagcctgtacaacattataaaacagctccaggaaaatgatgtgaagacatccagagattcaatgcttgaagaccctaaacatgtagcgctacacaacctgatactatcttatgttgatatgagtaaaaatccggcaaatttaatcaacagcctcaagcggcttcctagagaaaaactcaaaaagcttgccaagattatattacagctatctgcagggtcagagagtgacaatgccagtggtaataccctgcaaaagggtgacagcaataattaagtgcaatgcacttggattgtgtatggttcgtaagatatatgattatagtattgcaagctggagtgatttaatagaggaagtagccaatatgatcctaatagatcacattaataggaaacaatgtgtggagtgtcgaaaagattttgagtttatagctatatatacctcatataattagttaattaaaaccaattaagctataagtccaaagaagggtgggacaagtcataatgacttctaccgtcaatcttgggtcagacactgcttcaaaatggaaagtgatcaagtcaaggtgcaattcctgctgcaggatcttggtgagttgtgctgctgtgatttgtgcaatcttagctttgatcttccttgttgctacgatcggattgtctgtgaaactggcattcacagtgcaggagatccacaactgcaagcagaagctatcaggggcgagcacaactacagctgcaatctatactactccaagcacaatgatagaagcactacagacaaatcaacttaaactgacaaccaatgaaggacgtagtactctccctgattgcctggtggagaaaaaattgtgcgaaggtgaggtcaggtatctcaaaactaaaggctgcttgggggcacgagagggtgaagatttgaattgtatagatttagtagtggagtgtgttgggaaaccctgtggtcacaacgaggattacaaagaatgcatatgcaccaacaatgggacagcaactaagtgctgctacatctaatgaatattgacaacactagtgccaaataataggcaacagcattatttaattaaaaagaagaaagatcggaacaagtatctcaatggggtccaaactttatatggttcagggcaccagtgcatatcaaactgcagtggggttctggctaaatattgggaggaggtacatattggctatagtcctatcagctttcgggctgacctgcacagtcactattgcactcactgttagtgtcatagttgaacagtcagtgctagaagagtgcagaaactacaatggaggagatagagattggtggtcaaccacccagaagcagtcaactactgcaccaagtgcgactccaacaggaaattatggaggattacaaacggctcgaacaagaaagtctggaagctgtttgcatgtgcaaatttcttatggtgatatgtataaccgcagtaatactgtactgggtggttttgattgtatgggcttattggctctttgcaaatcaggaccgatttgtcagcgagataatcaagttgacccaacagtcctctgccactgcagggtaggtctttcaagtgtggactgctgcaaggtgaacaagatcagcaccaacagcaacaccacctctgagctccagaagaccaacccgacatggcatagccaagacaacacagactccaatccaaatccccaaagcataactaccagcacagccactctgctctcaacaggtctgggcctcatgctcacatcgaagaccgggacacacaaatcagggccctcccaagccctgccggggagcaacaccaacagaaaaacaaccacggaccgagaactagggtccacaaatcaaccaaattcaaccaataatgtgcaacatgataagcacacccaacgaacgacacccctgccaggctacaacaacacaagaactatcctccagcacacaacactctgggagaagacactcagcacatacaagcccacacactctttaaccaacgaatcagatcaacccctctccacaactcaaaacagcatcaactgtgaacattttaacccccaaggcagagaaaaaatctgctacagagtaggttcttacaactccaatattacaaagcaatgcagaattgatgtgcctttgtgttccacttataacacagtgtgcatgaaaacatactataccgaaccattcaactgttggaggcgtatctggcgctgcttgtgtgatgacggagttggactggttgagtggtgttgcactagttaactaaaaatgaaaacacatacatccgataatagaaaaatacatcagatagtctaaaacaattaaaagaatgaaaacaaggaccaatatggaaatatccgatgagtccgtcgtaaatgtctatctgcctgactcttacctaaagggggtcatttcattcagtgagaccaatgcaattggatcatgtgtactcaaccggccatacattaaagatgaccatacagcacatgttgcaatgactaacccagtgattgaacaccagaggttgagggccctcttcaagagtcttactattagcagagaatatcgagtggttgaacctcttatgatccagaaggagctgctaaaagtcgcagcaggtgcaagactaaagaaactcaagaaatggttgggtagaagcaaggatataagtgaagtcaaattgaaaatggtgactgattggcttaaactgtcacagacaccaggaaggggaaaaataatagattgtattcaagtggagaacttacctgactggctggagcactggtttgattcttggttgatattgaatgacgtcatccaatcttataggtgtttggaagtatcccaaaccagtgctatattacggaaaagtagcctgaacttcttttttgctgtttcttcttttgggtgtataatcataagtcgcaagagtagaagaatatgtttttgcacctacaatcaactcctgacttggaaggaccttgctttaagcagattcaatgcaaatttatgtgtgtgggtgagcaattgcttaaatagcgcacaagacggcctcggactaaggagcaaattagttggtgagttactcaaccgattgtactgtgagactgatgagttattgagcatcactggcaatgaggggtatgggattgtaaaggaatttgaaggattcattatgagtgaaatattgcggatgactgaacatgcacaattcagtgtaaggttccggaatacgctattgaatggcatagctgataaaatagggaagatgagagccgcataccaaagacggacacagaacacatctgttgagcatcatagatacccaagtgagcatgaaatgctggaagcaacagggagagtactgagaataattaagcttctggtgaataatgagattgctaatgctgcagagatgtattttatattccgcatattcggacatcctatggtagaagaaagggaggctatggatgcagtgagggataacagtgaaactgcaaaaatcttaagtctggtggcccttacggagatgaggggggccttcattctaagagtgatcaaaggtttcgtagggaactacaaaagatggccaaagataaagaatccatcaacattgagtcgaaggtggaagatgtacatgaatgcaaaaacatacccaagcaatcttgaattgtgtgtggaagacttcttggagcttgctgggatcagtttttgccaagaattctatgtacctagccagaccagtttggagatggtactgaatgataaagcaatatctccccccaaatctctcatatggtctgtgtatccacgtaactaccttccgcaaaacatacaagacagattcaggttggagtctcttgacagactagagcatgagcgcaccaggagagtgctagagttctacttaaaagatgctaattttaagcaaagtgatctcaaaaaatatgttgtaggtcaagagtacttgaatgacaaagagcatgtggtctccttaacagggaaagaaagggaactaagtgtgggtcgtatgtttgcgatgcaacctggaaagcaaagacaggtgcagatccttgctgagaaattgttatcagataacatagtgccatttttcccagagacattgactaggtatggagatctagaattacaaagaatcatggaactcaagtccgaactatcgtctacaaaaaaacggaaaagtgatagctacaataattatatagcaagagcttcaatagtgactgatttgagtaagtttaaccaagcttttagatatgagactacttcagtgtgtgctgatgttgcagatgaactgcatgggactcaaagccttttctgttggttacacctgactgtatcctccacaaccatgatctgcacttatagacatgctccccctgatacaggtggaatctacgacatagaccagataccggagcaaagcggactgtataggttccacatgggtggcatagaaggctggtgccaaaagatgtggaccatggaagcaatatcacttcttgatgtggtgtctgtgaggaatcgggtgcagctcacatcactgctgaatggggacaaccagtctatagatgtgagtaaacctgtgcgactcacaggtgctcaaactgaaattcaggctgattatagccttgccattaagatgttaactgctgtaagggatgcctactataacataggacacaagttgaaggagggagagacttatgtttcaagagacctccagtttatgagcaagaccatccaatcagaaggcgtaatgtatcctgcttcaattaagaaggttctgcgagtgggtccctggataaacacaatactggatgacatcaaaactagtatggaagctattgggagtctctgtcaagagctagaattccgaggggagtcatttgtgactagtcttataattcggaatttctggttgtacatccagtgctttgtcgaggcaaaaaatcatagcctagcaggagcacaaatatccaatgagcttaatagaactttaaccaaagttaagagattcttcaggttagcagatgagagcagcactgttaacttgttcatgaacataccaatgcagttagggggaggggaccctgttgttgtttataggagtttttatagacggacaccagattttttgacagaggtgttaacacacatggaactactactaacagcaatagagatggataggggagtttatatgaacttcttcttaacactgttgaacacatgcaaaaatgatagagccactcttaccacactgatgagagacccccaagctattgggtcagaaaggcaggctaaggtaacaagcgaaattaatagaacagcagtaactagtgtgctcagccttgcaccaaaccccttgtttagtgatagtgctgtgcatttcagtcagaatgaggaagaaataggcacagttatggaggatatatcacctgtctacccacatggactgagagtggtgtatgaagcattcccattccacaaagctgaaaaagttgtaaacatgattgctggaaccaagtccataacaaatatcctccaaagaacttctgctataagtggtgtagatattgacagagcagtccacatgatgttgctcaacttgggcttgttgggtagaatgcttgagtctggacccatcactgacacaatagatctgaggagcaacaatcggattctttgttgtcagctttccaaaagaataagagaaatgtcatgggatgggttggagattgtcggtgtatcatctccaagcatgttgtctagcttagatattaattatgttacaatagcacagaagccaggtatcattgtagaaaaattctcagctgaaaaaacaacaagagggaaaagaggtccgaaagcaccatgggtaggctcgagcacacaagagaaaaaattggtagctgtttacaacagacaggttttatcgaaagaacaaaaagaccaattagaaaccataggcaaaatcaggtgggtctacaaaggcgttactggcctccgacgtcttctaaacttaatatgcttaggaacactgggtcttccatataagctgttaaaaccacttctacctcggtttatgagcgtcaatttcctacacaggcttgcagtgagcagcaggcctatggagttcccatcatcagtaccggcatataggactacaaactttcattttgatactagtcctataaacaggcggttgagtgagaggtttggcaatgaggacatcaaccttgtgttccaaaatgctataagttgtggaattagtgtcatgtgtgtggttgaacaattgaccgggcgtagccctaaacaagtgatactagaacctattgtagaagatattgacataatgtccgcccctaatttccaaggccgcctgaatttcaagagtgtaagaaagattgtgactgatcaacatatctttaatccggaccacattgacttggttatgcttgggaagttgttattaccaactgtccgtagcaatataaataacaacaaactagcaactgaaaatttctttaatggtaacaacatagtagaggcactaacaagctgtcttgcttgccactggtgcactgttttgattctactcactactgagaatagtattttccagaaagagtggggagatggattcataacagaccatgccttcataaacttcacatggttccttatgagtttcaaaacttatctcctatgccattggcaaactgaaaagaacaagacattggggattgtagaggatcccatagacaggctcatcagagtagacaatagtttttggcggatgatgagcaaggtgttcctggaacccaaagtgaaaagaagacttatgctttacgatacgtctgtattgaatgttctgggtagtatcagtttcaaaaactggtttgttagccggttaagaacagtagactacatggagatcccatggattgtcaatgctgagggtgacatagttgagcaaaaacctgtcaatgaatatctgaaaataatggctgcaggtgtccatgtaaaagtcataatgcttagttactccgatatggcacatgctatgacacgcttgctaaggtgcaagaacatgcaagacaatgtgcccaccataaagaaaacagtatctccaactgatgtcacaccctctgtagaccctactcgggccctactgctgttcccgaaagtcatctttagtaagttgaccacttttaatgcaacatgcagtacagctaagcaatctggcagcaatttgacaaaaaactacatgactttaatgccttggcaccacattaatcgttataactttgtccatagctccacagggtgtaaggtaagtgtaaagggatgtataggcaagttgataacaaaacttgaattaagaattatctacttcatcggtgagggagctgggaatctcatgtccagaactgcatgtgaataccctaatctaaagtttgtatacaggagtctgaaagatgccaatgatcatcatcatcccacagagtatgtgcgtgtaatggggagcatcagcagaataattgacctcggtgaagggttggcaatggaagcaactgatgcaactaagagagaacattgggattttatccacagagtgtgcaaagatcctcttctgctaactatctgtgatgctgaactaaaaaacatggaggatgtcctgcatttagtgatacagtggaggaaacatgtactatcttgtaacatatgcacatcatatgggactaatgtgtacatgtttgttaagtatcatgcgcaatctgaggcaaagaagctaccacattttgtccgagtagtttcaatatatatcatgcaagggagtaaactatcaggatcagaatgttacatgttgttaacattaggacaccagaacatggcaccttgctatggagaggttaggtctgcgaaatcaatcatggctttgacgagcacttttaaacacccctgtagactagacaaatctgccattgaggccaacctgaaatcactagcacccgggttgacactgccacttagtgagcctgctatgcaaaagtacttgttggatctttcaagcttaagtggaattaagaacacgggggcaaccattgggggaagcaaagtagtagagaagaagtgggctttatgcaaagtgtcaaatatagtgaggtggttaagtcacgttctgaaaatgcccaaaggtgatttaaattacgattttttcgaggtgatagaaaacacatacccagacatggtgaagctattggacaacttaaccccgtcagagttgaaaaagctcgtcaaaatcaccgggtacatcctgggcacaaagtaataaggcaatatctactcaacacataattatcacctgaaacacaactgacc

SEQ ID NO: 5 - RNA, 13302 nt, Avian metapneumovirus
  source 1..13302: mol_type = genomic RNA
catttaagcaggttttagaaaaacaatgggacaagtcaaaaatgtctcttgaaagcattagactcagtgacttggagtacaaacatgcaatacttgaagactctcagtatacaattagaagggatgttggtgctaccactgcaattacaccttctgaactgcagccgcaagtatccatattatgtggtatggtgttgtttgcaaaacacaccgactatgagcctgcagcagaggtaggcatgcagtacattagtactgctctaggagctgatagaactcaacaaatactgaaaaattccggtagtgaagtacagggtgttatgaccaagattgtaacacttccagcagagggtcctgtcagaaagcgagaagtgctaaacattcatgatgtaggtgttgggtgggctgatgatgttgaaaggactgcaagagaagcaatgggagcgatggttagagaaaaagtgcaactcacaaagaatcaaaagccgtctgccttggatgctcccgttattctattatgcattggtgccctcattttcaccaagttggcctcaaccgttgaagtaggtcttgaaactgctatccgacgtgcctcaagggtattaagcgatgccatatcacggtaccccaggatggatataccaaggattgccaaatcattctttgaattgtttgagaagaaggtgtattacaggaatttatttattgaatacggtaaggcactcggaggtacatccaccggaagcaggatggagagcctgtttgtgaatatttttatgcaagcttatggggcagggcaaacaatgctgcgctggggtgtcattgcacgatcttccaacaatataatgttaggccatgtatctgtacaggctgagttaaggcaagtatctgaggtctatgacctggtgaggaaaatgggacctgagtcaggattactacacttacgccagagtcccaaagcgggtcttttgtcgttgaccaactgtcccaattttgccagtgttgtcctcgggaacgccgccgggcttggtattataggcatgtacaaaggtcgagcccccaaccttgagctgtttgctgctgctgaaagttatgcacgaacattgaaagagaacaacaagatcaacctagcagccttagggctcactgatgatgagagggaagctgcaacatcctacctagggggagatgatgagagatcatctaagtttgagtaattaaaaaatatgggacaagtaacaatgtctttccctgaaggcaaggatattctaatgatgggcagcgaggctgcaaaaatggccgatgcctatcaaagatcactaaggaacacctctgctggaggccgctctattagtggagaacctattaacacaattgctgaaaaggtgccattaccccctttgtgcaaccccaccacaccaaggggatcatgtataaaacccaacaaggctcctgtctccaaagtcaaagaaatagaatctatatatccaaaactcccaactgctccagttgccactgatactgagacttctaccagtactgagtctgcaaagaagtctaaaaaggtcaagtttgacaaccctaaggtaggaaaatatactaaactggaagaagaggcactagagcttttgtcagatcctgaagaggataatgatgaaaagtcatcaatattgacatttgaggaaaaagatacagcatctacctctattgaagcaagattagaagcaatagaagaaaagttgagcatgatattgggcatgctcaaaacattgaacattgcaactgctggcccaaccgccgccagagatggaatacgtgatgcaatgatagggatgagagaagagctgataaacagcattatgacagaggctaaagacaaaattgcagaaatgatgaaagaagaggatacccagagggcgaaaataggagacggcagcgtgaagcttaccgaaaaagctaaagagttgaacaaagtcctagaagaccaaagctcaagtggagagtcagagagtgaagaagagagtggagagtctgactttaatgaggaagagagtgacatatacaatcttgatctgtagttatgaaaaaatgggacaagtcaaaatggagtcctatataattgacacttatcagggggttccctacactgcagccgttcaggttgacctcattgaaaaagactctaaccctgcaacattgactgtatggtttcccctattccagtcaagtacgcctgccccggtgttattagatcaactaaaaactttatcaatcaccacccagtacacagcatctccggaaggccctgtgctccaagtcaatgcagcagcccaaggtgccgccatgtctgcactacctaagaaatttgccgttagtgctgcagtagcacttgatgagtacagcagactggaattcgggacattgactgtgtgtgatgtgagatccatttatctgactaccttgaagccatatggcatggtctccaaaataatgacagatgtgaggtctgttgggaggaagacccatgacttgatcgcattgtgtgattttatagacatagagaaaggggtgcccataaccatacctgcatatattaaagctgtctctatcaaagactcagaatcagccactgtggaagctgcaataagtggagaagcagaccaggctataactcaagcaaggatagctccatatgcagggctgatcttgattatgactatgaacaaccctaaagggatctttaagaaattgggtgcaggtatgcaagtgatagtagaactggggccgtatgtacaagcagagagtctgggaaaaatctgcaagacatggaatcatcaacgaaccaggtatgtactgaggtcacgttgagggtaccctaaagcataaatctgttaatgtcatagtcaataaaaaataaaaaattgggacaagtaggatggatgtaagaatctgcctcctattgttccttatatccaatccgggtagctgcatacaagaaacatataatgaagaatcttgcagtactgtaactagaggctataagagtgtgttaagaacagggtggtatacgaatgtatttaaccttgaaatagggagtgttgagaacatcacttgcaatgatggacccagcctaattgacactgagttggtactcacaaagaatgctttgagggaactcaaaacagtgtcagctgatcaagtggccaaggaaagcagactatcctcacccaggagacgtagatttgtactgggtgcaattgcacttggcgttgcaacggctgctgccgtaacagctggtgtagcacttgcaaagacaattagattagagggagaggtgaaggcaatcaagaatgccctccggaacacaaatgaggcagtatccacattagggaatggtgtgagggtactagcaactgcagtcaatgacctcaaggaatttataagtaaaaaattgactcctgctattaaccggaacaaatgcaatatagcagatataaagatggcaattagttttggccaaaataacagaaggttcttgaatgtggtgaggcaattctctgatagtgcaggtatcacatcagctgtgtctcttgatttaatgacagatgatgaacttgttagagcaattaacaaaatgccaacttcatcaggacagattagtttgatgttgaacaatcgttcaatggttagaaggaaggggttcggtatattgattggtgtttatgatggaacggtcgtttatatggtacaactgcccatattcggcgtgattgagacaccttgttggagggtggtggcagcaccactctgtagggaagagaaaggcaattatgcttgtatactaagagaagatcaagggtggtactgtacaaatgctggctctacagcttattaccctaatgaagatgattgtgaagtaagggatgactatgtgttttgtgacacagcagctggcattaatgtagcctcagaagttgagcagtgcaactataacatatcgacttctaaatacccatgcaaagtcagcacaggtaggcaccccgtcagtatggtagctttaacccccctagggggtctagtgtcttgttatgagggtgtaagttgctccataggcagcaataaagtagggataataaaacagctaggcaaagggtgcacccacattcccaacaacgaagctgacacaataaccattgataacactgtgtaccaattgagcaaggttgtaggtgaacagaggaccataaaaggagccccagttgtgaacaattttaatccaatattattccctgaggatcagttcaatgttgcacttgaccaagtatttgagagtatagatagatctcaagacttaatagataagtctaacgacttgctaggtgcagatgccaagagcaaggctggaattgctatagcaatagtagtgctagtcattctaggaatcttctttgtgcttgcagtgatatattactgtttcagagtccagaagaccaaaccaaagcatgattatccggccacgacaggtcatagcagcatggcttatgtcagttaagttatttaaaattgggacaagtgaggatgtctaggcgaaatccctgcagatatgaaatacggggcaaatgcaacaggggatcatcctgcacgtttaaccataattattggtcctggcctgatcatgtgctcttagtcagagcaaactacatgcttaaccaattagtgaggaacacagacaggacagatggcctatccttgatatcaggggcaggacgagaagatagaacccaggattttgttctaggatctgccaatgttgtacagaattacatagaagggaacacaacaatcaccaaatcatcagcctgttacagcctgtacaacattataaaacagctccaggaaaatgatgtgaagacatccagagattcaatgcttgaagaccctaaacatgtagcgctacacaacctgatactatcttatgttgatatgagtaaaaatccggcaaatttaatcaacagcctcaagcggcttcctagagaaaaactcaaaaagcttgccaagattatattacagctatctgcagggtcagagagtgacaatgccagtggtaataccctgcaaaagggtgacagcaataattaagtgcaatgcacttggattgtgtatggttcgtaagatatatgattatagtattgcaagctggagtgatttaatagaggaagtagccaatatgatcctaatagatcacattaataggaaacaatgtgtggagtgtcgaaaagattttgagtttatagctatatatacctcatataattagttaattaaaaccaattaagctataagtccaaagaagggtgggacaagtcataatgacttctaccgtcaatcttgggtcagacactgcttcaaaatggaaagtgatcaagtcaaggtgcaattcctgctgcaggatcttggtgagttgtgctgctgtgatttgtgcaatcttagctttgatcttccttgttgctacgatcggattgtctgtgaaactggcattcacagtgcaggagatccacaactgcaagcagaagctatcaggggcgagcacaactacagctgcaatctatactactccaagcacaatgatagaagcactacagacaaatcaacttaaactgacaaccaatgaaggacgtagtactctccctgattgcctggtggagaaaaaattgtgcgaaggtgaggtcaggtatctcaaaactaaaggctgcttgggggcacgagagggtgaagatttgaattgtatagatttagtagtggagtgtgttgggaaaccctgtggtcacaacgaggattacaaagaatgcatatgcaccaacaatgggacagcaactaagtgctgctacatctaatgaatattgacaacactagtgccaaataataggcaacagcattatttaattaaaaagaagaaagatcgggacaagtatctcaatggggtccaaactttatatggttcagggcaccagtgcatatcaaactgcagtggggttctggctaaatattgggaggaggtacatattggctatagtcctatcagctttcgggctgacctgcacagtcactattgcactcactgttagtgtcatagttgaacagtcagtgctagaagagtgcagaaactacaatggaggagatagagattggtggtcaaccacccagaagcagtcaactactgcaccaagtgcgactccaacaggaaattatggaggattacaaacggctcgaacaagaaagtctggaagctgtttgcatgtgcaaatttcttatggtgatatgtataaccgcagtaatactgtactgggtggttttgattgtatgggcttattggctctttgcaaatcaggaccgatttgtcagcgagataatcaagttgacccaacagtcctctgccactgcagggtaggtctttcaagtgtggactgctgcaaggtgaacaagatcagcaccaacagcaacaccacctctgagctccagaagaccaacccgacatggcatagccaagacaacacagactccaatccaaatccccaaagcataactaccagcacagccactctgctctcaacaggtctgggcctcatgctcacatcgaagaccgggacacacaaatcagggccctcccaagccctgccggggagcaacaccaacagaaaaacaaccacggaccgagaactagggtccacaaatcaaccaaattcaaccaataatgtgcaacatgataagcacacccaacgaacgacacccctgccaggctacaacaacacaagaactatcctccagcacacaacactctgggagaagacactcagcacatacaagcccacacactctttaaccaacgaatcagatcaacccctctccacaactcaaaacagcatcaactgtgaacattttaacccccaaggcagagaaaaaatctgctacagagtaggttcttacaactccaatattacaaagcaatgcagaattgatgtgcctttgtgttccacttataacacagtgtgcatgaaaacatactataccgaaccattcaactgttggaggcgtatctggcgctgcttgtgtgatgacggagttggactggttgagtggtgttgcactagttaactaaaaatgaaaacacatacatccgataatagaaaaatacatcagatagtctaaaacaattaaaagaatgaaaacaaggaccaatatggaaatatccgatgagtccgtcgtaaatgtctatctgcctgactcttacctaaagggggtcatttcattcagtgagaccaatgcaattggatcatgtgtactcaaccggccatacattaaagatgaccatacagcacatgttgcaatgactaacccagtgattgaacaccagaggttgagggccctcttcaagagtcttactattagcagagaatatcgagtggttgaacctcttatgatccagaaggagctgctaaaagtcgcagcaggtgcaagactaaagaaactcaagaaatggttgggtagaagcaaggatataagtgaagtcaaattgaaaatggtgactgattggcttaaactgtcacagacaccaggaaggggaaaaataatagattgtattcaagtggagaacttacctgactggctggagcactggtttgattcttggttgatattgaatgacgtcatccaatcttataggtgtttggaagtatcccaaaccagtgctatattacggaaaagtagcctgaacttcttttttgctgtttcttcttttgggtgtataatcataagtcgcaagagtagaagaatatgtttttgcacctacaatcaactcctgacctggaaggaccttgctttaagcagattcaatgcaaatttatgtgtgtgggtgagcaattgcttaaatagcgcacaagacggcctcggactaaggagcaaattagttggtgagttactcaaccgattgtactgtgagactgatgagttattgagcatcactggcaatgaggggtatgggattgtaaaggaatttgaaggattcattatgagtgaaatattgcggatgactgaacatgcacaattcagtgtaaggttccggaatacgctattgaatggcatagctgataaaatagggaagatgagagccgcataccaaagacggacacagaacacatctgttgagcatcatagatacccaagtgagcatgaaatgctggaagcaacagggagagtactgagaataattaagcttctggtgaataatgagattgctaatgctgcagagatgtattttatattccgcatattcggacatcctatggtagaagaaagggaggctatggatgcagtgagggataacagtgaaactgcaaaaatcttaagtctggtggcccttacggagatgaggggggccttcattctaagagtgatcaaaggtttcgtagggaactacaaaagatggccaaagataaagaatccatcaacattgagtcgaaggtggaagatgtacatgaatgcaaaaacatacccaagcaatcttgaattgtgtgtggaagacttcttggagcttgctgggatcagtttttgccaagaattctatgtacctagccagaccagtttggagatggtactgaatgataaagcaatatctccccccaaatctctcatatggtctgtgtatccacgtaactaccttccgcaaaacatacaagacagattcaggttggagtctcttgacagactagagcatgagcgcaccaggagagtgctagagttctacttaaaagatgctaattttaagcaaagtgatctcaaaaaatatgttgtaggtcaagagtacttgaatgacaaagagcatgtggtctccttaacagggaaagaaagggaactaagtgtgggtcgtatgtttgcgatgcaacctggaaagcaaagacaggtgcagatccttgctgagaaattgttatcagataacatagtgccatttttcccagagacattgactaggtatggagatctagaattacaaagaatcatggaactcaagtccgaactatcgtctacaaaaaaacggaaaagtgatagctacaataattatatagcaagagcttcaatagtgactgatttgagtaagtttaaccaagcttttagatatgagactacttcagtgtgtgctgatgttgcagatgaactgcatgggactcaaagccttttctgttggttacacctgactgtatcctccacaaccatgatctgcacttatagacatgctccccctgatacaggtggaatctacgacatagaccagataccggagcaaagcggactgtataggttccacatgggtggcatagaaggctggtgccaaaagatgtggaccatggaagcaatatcacttcttgatgtggtgtctgtgaggaatcgggtgcagctcacatcactgctgaatggggacaaccagtctatagatgtgagtaaacctgtgcgactcacaggtgctcaaactgaaattcaggctgattatagccttgccattaagatgttaactgctgtaagggatgcctactataacataggacacaagttgaaggagggagagacttatgtttcaagagacctccagtttatgagcaagaccatccaatcagaaggcgtaatgtatcctgcttcaattaagaaggttctgcgagtgggtccctggataaacacaatactggatgacatcaaaactagtatggaagctattgggagtctctgtcaagagctagaattccgaggggagtcatttgtgactagtcttataattcggaatttctggttgtacatccagtgctttgtcgaggcaaaaaatcatagcctagcaggagcacaaatatccaatgagcttaatagaactttaaccaaagttaagagattcttcaggttagcagatgagagcagcactgttaacttgttcatgaacataccaatgcagttagggggaggggaccctgttgttgtttataggagtttttatagacggacaccagattttttgacagaggtgttaacacacatggaactactactaacagcaatagagatggataggggagtttatatgaacttcttcttaacactgttgaacacatgcaaaaatgatagagccactcttaccacactgatgagagacccccaagctattgggtcagaaaggcaggctaaggtaacaagcgaaattaatagaacagcagtaactagtgtgctcagccttgcaccaaaccccttgtttagtgatagtgctgtgcatttcagtcagaatgaggaagaaataggcacagttatggaggatatatcacctgtctacccacatggactgagagtggtgtatgaagcattcccattccacaaagctgaaaaagttgtaaacatgattgctggaaccaagtccataacaaatatcctccaaagaacttctgctataagtggtgtagatattgacagagcagtccacatgatgttgctcaacttgggcttgttgggtagaatgcttgagtctggacccatcactgacacaatagatctgaggagcaacaatcggattctttgttgtcagctttccaaaagaataagagaaatgtcatgggatgggttggagattgtcggtgtatcatctccaagcatgttgtctagcttagatattaattatgttacaatagcacagaagccaggtatcattgtagaaaaattctcagctgaaaaaacaacaagagggaaaagaggtccgaaagcaccatgggtaggctcgagcacacaagagaaaaaattggtagctgtttacaacagacaggttttatcgaaagaacaaaaagaccaattagaaaccataggcaaaatcaggtgggtctacaaaggcgttactggcctccgacgtcttctaaacttaatatgcttaggaacactgggtcttccatataagctgttaaaaccacttctacctcggtttatgagcgtcaatttcctacacaggcttgcagtgagcagcaggcctatggagttcccatcatcagtaccggcatataggactacaaactttcattttgatactagtcctataaacaagcggttgagtgagaggtttggcaatgaggacatcaaccttgtgttccaaaatgctataagttgtggaattagtgtcatgtgtgtggttgaacaattgaccgggcgtagccctaaacaagtgatactagaacctattgtagaagatattgacataatgtccgcccctaatttccaaggccgcctgaatttcaagagtgtaagaaagattgtgactgatcaacatatctttaatccggaccacattgacttggttatgcttgggaagttgttattaccaactgtccgtagcaatataaataacaacaaactagcaactgaaaatttctttaatggtaacaacatagtagaggcactaacaagctgtcttgcttgccactggtgcactgttttgattctactcactactgagaatagtattttccagaaagagtggggagatggattcataacagaccatgccttcataaacttcacatggttccttatgagtttcaaaacttatctcctatgccattggcaaactgaaaagaacaagacattggggattgtagaggatcccatagacaggctcatcagagtagacaatagtttttggcggatgatgagcaaggtgttcctggaacccaaagtgaaaagaagacttatgctttacgatacgtctgtattgaatgttctgggtagtatcagtttcaaaaactggtttgttagccggttaagaacagtagactacatggagatcccatggattgtcaatgctgagggtgacatagttgagcaaaaacctgtcaatgaatatctgaaaataatggctgcaggtgtccatgtaaaagtcataatgcttagttactccgatatggcacatgctatgacacgcttgctaaggtgcaagaacatgcaagacaatgtgcccaccataaagaaaacagtatctccaactgatgtcacaccctctgtagaccctactcgggccctactgctgttcccgaaagtcatctttagtaagttgaccacttttaatgcaacatgcagtacagctaagcaatctggcagcaatttgacaaaaaactacatgactttaatgccttggcaccacattaatcgttataactttgtccatagctccacagggtgtaaggtaagtgtaaagggatgtataggcaagttgataacaaaacttgaattaagaattatctacttcatcggtgagggagctgggaatctcatgtccagaactgcatgtgaataccctaatctaaagtttgtatacaggagtctgaaagatgccaatgatcatcatcatcccacagagtatgtgcgtgtaatggggagcatcagcagaataattgacctcggtgaagggttggcaatggaagcaactgatgcaactaagagagaacattgggattttatccacagagtgtgcaaagatcctcttctgctaactatctgtgatgctgaactaaaaaacatggaggatgtcctgcatttagtgatacagtggaggaaacatgtactatcttgtaacatatgcacatcatatgggactaatgtgtacatgtttgttaagtatcatgcgcaatctgaggcaaagaagctaccacattttgtccgagtagtttcaatatatatcatgcaagggagtaaactatcaggatcagaatgttacatgttgttaacattaggacaccagaacatggcaccttgctatggagaggttaggtctgcgaaatcaatcatggctttgacgagcacttttaaacacccctgtagactagacaaatctgccattgaggccaacctgaaatcactagcacccgggttgacactgccacttagtgagcctgctatgcaaaagtacttgttggatctttcaagcttaagtggaattaagaacacgggggcaaccattgggggaagcaaagtagtagagaagaagtgggctttatgcaaagtgtcaaatatagtgaggtggttaagtcacgttctgaaaatgcccaaaggtgatttaaattacgattttttcgaggtgatagaaaacacatacccagacatggtgaagctattggacaacttaaccccgtcagagttgaaaaagctcgtcaaaatcaccgggtacatcctgggcacaaagtaataaggcaatatctactcaacacataattatcacctgaaacacaactgacc

SEQ ID NO: 6 - RNA, 13302 nt, Avian metapneumovirus; identical to SEQ ID NO: 5
  source 1..13302: mol_type = genomic RNA

SEQ ID NO: 7 - RNA, 13302 nt, Avian metapneumovirus
  source 1..13302: mol_type = genomic RNA
catttaagcaggttttagaaaaacaatgggacaagtcaaaaatgtctcttgaaagcattagactcagtgacttggagtacaaacatgcaatacttgaagactctcagtatacaattagaagggatgttggtgctaccactgcaattacaccttctgaactgcagccgcaagtatccatattatgtggtatggtgttgtttgcaaaacacaccgactatgagcctgcagcagaggtaggcatgcagtacattagtactgctctaggagctgatagaactcaacaaatactgaaaaattccggtagtgaagtacagggtgttatgaccaagattgtaacacttccagcagagggtcctgtcagaaagcgagaagtgctaaacattcatgatgtaggtgttgggtgggctgatgatgttgaaaggactgcaagagaagcaatgggagcgatggttagagaaaaagtgcaactcacaaagaatcaaaagccgtctgccttggatgctcccgttattctattatgcattggtgccctcattttcaccaagttggcctcaaccgttgaagtaggtcttgaaactgctatccgacgtgcctcaagggtattaagcgatgccatatcacggtaccccaggatggatataccaaggattgccaaatcattctttgaattgtttgagaagaaggtgtattacaggaatttatttattgaatacggtaaggcactcggaggtacatccaccggaagcaggatggagagcctgtttgtgaatatttttatgcaagcttatggggcagggcaaacaatgctgcgctggggtgtcattgcacgatcttccaacaatataatgttaggccatgtatctgtacaggctgagttaaggcaagtatctgaggtctatgacctggtgaggaaaatgggacctgagtcaggattactacacttacgccagagtcccaaagcgggtcttttgtcgttgaccaactgtcccaattttgccagtgttgtcctcgggaacgccgccgggcttggtattataggcatgtacaaaggtcgagcccccaaccttgagctgtttgctgctgctgaaagttatgcacgaacattgaaagagaacaacaagatcaacctagcagccttagggctcactgatgatgagagggaagctgcaacatcctacctagggggagatgatgagagatcatctaagtttgagtaattaaaaaatatgggacaagtaacaatgtctttccctgaaggcaaggatattctaatgatgggcagcgaggctgcaaaaatggccgatgcctatcaaagatcactaaggaacacctctgctggaggccgctctattagtggagaacctattaacacaattgctgaaaaggtgccattaccccctttgtgcaaccccaccacaccaaggggatcatgtataaaacccaacaaggctcctgtctccaaagtcaaagaaatagaatctatatatccaaaactcccaactgctccagttgccactgatactgagacttctaccagtactgagtctgcaaagaagtctaaaaaggtcaagtttgacaaccctaaggtaggaaaatatactaaactggaagaagaggcactagagcttttgtcagatcctgaagaggataatgatgaaaagtcatcaatattgacatttgaggaaaaagatacagcatctacctctattgaagcaagattagaagcaatagaagaaaagttgagcatgatattgggcatgctcaaaacattgaacattgcaactgctggcccaaccgccgccagagatggaatacgtgatgcaatgatagggatgagagaagagctgataaacagcattatgacagaggctaaagacaaaattgcagaaatgatgaaagaagaggatacccagagggcgaaaataggagacggcagcgtgaagcttaccgaaaaagctaaagagttgaacaaagtcctagaagaccaaagctcaagtggagagtcagagagtgaagaagagagtggagagtctgactttaatgaggaagagagtgacatatacaatcttgatctgtagttatgaaaaaatgggacaagtcaaaatggagtcctatataattgacacttatcagggggttccctacactgcagccgttcaggttgacctcattgaaaaagactctaaccctgcaacattgactgtatggtttcccctattccagtcaagtacgcctgccccggtgttattagatcaactaaaaactttatcaatcaccacccagtacacagcatctccggaaggccctgtgctccaagtcaatgcagcagcccaaggtgccgccatgtctgcactacctaagaaatttgccgttagtgctgcagtagcacttgatgagtacagcagactggaattcgggacattgactgtgtgtgatgtgagatccatttatctgactaccttgaagccatatggcatggtctccaaaataatgacagatgtgaggtctgttgggaggaagacccatgacttgatcgcattgtgtgattttatagacatagagaaaggggtgcccataaccatacctgcatatattaaagctgtctctatcaaagactcagaatcagccactgtggaagctgcaataagtggagaagcagaccaggctataactcaagcaaggatagctccatatgcagggctgatcttgattatgactatgaacaaccctaaagggatctttaagaaattgggtgcaggtatgcaagtgatagtagaactggggccgtatgtacaagcagagagtctgggaaaaatctgcaagacatggaatcatcaacgaaccaggtatgtactgaggtcacgttgagggtaccctaaagcataaatctgttaatgtcatagtcaataaaaaataaaaaattgggacaagtaggatggatgtaagaatctgcctcctattgttccttatatccaatccgggtagctgcatacaagaaacatataatgaagaatcttgcagtactgtaactagaggctataagagtgtgttaagaacagggtggtatacgaatgtatttaaccttgaaatagggagtgttgagaacatcacttgcaatgatggacccagcctaattgacactgagttggtactcacaaagaatgctttgagggaactcaaaacagtgtcagctgatcaagtggccaaggaaagcagactatcctcacccaggagacgtagatttgtactgggtgcaattgcacttggcgttgcaacggctgctgccgtaacagctggtgtagcacttgcaaagacaattagattagagggagaggtgaaggcaatcaagaatgccctccggaacacaaatgaggcagtatccacattagggaatggtgtgagggtactagcaactgcagtcaatgacctcaaggaatttataagtaaaaaattgactcctgctattaaccggaacaaatgcaatatagcagatataaagatggcaattagttttggccaaaataacagaaggttcttgaatgtggtgaggcaattctctgatagtgcaggtatcacatcagctgtgtctcttgatttaatgacagatgatgaacttgttagagcaattaacaaaatgccaacttcatcaggacagattagtttgatgttgaacaatcgttcaatggttagaaggaaggggttcggtatattgattggtgtttatgatggaacggtcgtttatatggtacaactgcccatattcggcgtgattgagacaccttgttggagggtggtggcagcaccactctgtagggaagagaaaggcaattatgcttgtatactaagagaagatcaagggtggtactgtacaaatgctggctctacagcttattaccctaatgaagatgattgtgaagtaagggatgactatgtgttttgtgacacagcagctggcattaatgtagcctcagaagttgagcagtgcaactataacatatcgacttctaaatacccatgcaaagtcagcacaggtaggcaccccgtcagtatggtagctttaacccccctagggggtctagtgtcttgttatgagggtgtaagttgctccataggcagcaataaagtagggataataaaacagctaggcaaagggtgcacccacattcccaacaacgaagctgacacaataaccattgataacactgtgtaccaattgagcaaggttgtaggtgaacagaggaccataaaaggagccccagttgtgaacaattttaatccaatattattccctgaggatcagttcaatgttgcacttgaccaagtatttgagagtatagatagatctcaagacttaatagataagtctaacgacttgctaggtgcagatgccaagagcaaggctggaattgctatagcaatagtagtgctagtcattctaggaatcttctttgtgcttgcagtgatatattactgtttcagagtccagaagaccaaaccaaagcatgattatccggccacgacaggtcatagcagcatggcttatgtcagttaagttatttaaaattgggacaagtgaggatgtctaggcgaaatccctgcagatatgaaatacggggcaaatgcaacaggggatcatcctgcacgtttaaccataattattggtcctggcctgatcatgtgctcttagtcagagcaaactacatgcttaaccaattagtgaggaacacagacaggacagatggcctatccttgatatcaggggcaggacgagaagatagaacccaggattttgttctaggatctgccaatgttgtacagaattacatagaagggaacacaacaatcaccaaatcatcagcctgttacagcctgtacaacattataaaacagctccaggaaaatgatgtgaagacatccagagattcaatgcttgaagaccctaaacatgtagcgctacacaacctgatactatcttatgttgatatgagtaaaaatccggcaaatttaatcaacagcctcaagcggcttcctagagaaaaactcaaaaagcttgccaagattatattacagctatctgcagggtcagagagtgacaatgccagtggtaataccctgcaaaagggtgacagcaataattaagtgcaatgcacttggattgtgtatggttcgtaagatatatgattatagtattgcaagctggagtgatttaatagaggaagtagccaatatgatcctaatagatcacattaataggaaacaatgtgtggagtgtcgaaaagattttgagtttatagctatatatacctcatataattagttaattaaaaccaattaagctataagtccaaagaagggtgggacaagtcataatgacttctaccgtcaatcttgggtcagacactgcttcaaaatggaaagtgatcaagtcaaggtgcaattcctgctgcaggatcttggtgagttgtgctgctgtgatttgtgcaatcttagctttgatcttccttgttgctacgatcggattgtctgtgaaactggcattcacagtgcaggagatccacaactgcaagcagaagctatcaggggcgagcacaactacagctgcaatctatactactccaagcacaatgatagaagcactacagacaaatcaacttaaactgacaaccaatgaaggacgtagtactctccctgattgcctggtggagaaaaaattgtgcgaaggtgaggtcaggtatctcaaaactaaaggctgcttgggggcacgagagggtgaagatttgaattgtatagatttagtagtggagtgtgttgggaaaccctgtggtcacaacgaggattacaaagaatgcatatgcaccaacaatgggacagcaactaagtgctgctacatctaatgaatattgacaacactagtgccaaataataggcaacagcattaattaattaaaaagaagaaagatcgggacaagtatctcaatggggtccaaactttatatggttcagggcaccagtgcatatcaaactgcagtggggttctggctaaatattgggaggaggtacatattggctatagtcctatcagctttcgggctgacctgcacagtcactattgcactcactgttagtgtcatagttgaacagtcagtgctagaagagtgcagaaactacaatggaggagatagagattggtggtcaaccacccagaagcagtcaactactgcaccaagtgcgactccaacaggaaattatggaggattacaaacggctcgaacaagaaagtctggaagctgtttgcatgtgcaaatttcttatggtgatatgtataaccgcagtaatactgtactgggtggttttgattgtatgggcttattggctctttgcaaatcaggaccgatttgtcagcgagataatcaagttgacccaacagtcctctgccactgcagggtaggtctttcaagtgtggactgctgcaaggtgaacaagatcagcaccaacagcaacaccacctctgagctccagaagaccaacccgacatggcatagccaagacaacacagactccaatccaaatccccaaagcataactaccagcacagccactctgctctcaacaggtctgggcctcatgctcacatcgaagaccgggacacacaaatcagggccctcccaagccctgccggggagcaacaccaacagaaaaacaaccacggaccgagaactagggtccacaaatcaaccaaattcaaccaataatgtgcaacatgataagcacacccaacgaacgacacccctgccaggctacaacaacacaagaactatcctccagcacacaacactctgggagaagacactcagcacatacaagcccacacactctttaaccaacgaatcagatcaacccctctccacaactcaaaacagcatcaactgtgaacattttaacccccaaggcagagaaaaaatctgctacagagtaggttcttacaactccaatattacaaagcaatgcagaattgatgtgcctttgtgttccacttataacacagtgtgcatgaaaacatactataccgaaccattcaactgttggaggcgtatctggcgctgcttgtgtgatgacggagttggactggttgagtggtgttgcactagttaactaaaaatgaaaacacatacatccgataatagaaaaatacatcagatagtctaaaacaattaaaagaatgaaaacaaggaccaatatggaaatatccgatgagtccgtcgtaaatgtctatctgcctgactcttacctaaagggggtcatttcattcagtgagaccaatgcaattggatcatgtgtactcaaccggccatacattaaagatgaccatacagcacatgttgcaatgactaacccagtgattgaacaccagaggttgagggccctcttcaagagtcttactattagcagagaatatcgagtggttgaacctcttatgatccagaaggagctgctaaaagtcgcagcaggtgcaagactaaagaaactcaagaaatggttgggtagaagcaaggatataagtgaagtcaaattgaaaatggtgactgattggcttaaactgtcacagacaccaggaaggggaaaaataatagattgtattcaagtggagaacttacctgactggctggagcactggtttgattcttggttgatattgaatgacgtcatccaatcttataggtgtttggaagtatcccaaaccagtgctatattacggaaaagtagcctgaacttcttttttgctgtttcttcttttgggtgtataatcataagtcgcaagagtagaagaatatgtttttgcacctacaatcaactcctgacctggaaggaccttgctttaagcagattcaatgcaaatttatgtgtgtgggtgagcaattgcttaaatagcgcacaagacggcctcggactaaggagcaaattagttggtgagttactcaaccgattgtactgtgagactgatgagttattgagcatcactggcaatgaggggtatgggattgtaaaggaatttgaaggattcattatgagtgaaatattgcggatgactgaacatgcacaattcagtgtaaggttccggaatacgctattgaatggcatagctgataaaatagggaagatgagagccgcataccaaagacggacacagaacacatctgttgagcatcatagatacccaagtgagcatgaaatgctggaagcaacagggagagtactgagaataattaagcttctggtgaataatgagattgctaatgctgcagagatgtattttatattccgcatattcggacatcctatggtagaagaaagggaggctatggatgcagtgagggataacagtgaaactgcaaaaatcttaagtctggtggcccttacggagatgaggggggccttcattctaagagtgatcaaaggtttcgtagggaactacaaaagatggccaaagataaagaatccatcaacattgagtcgaaggtggaagatgtacatgaatgcaaaaacatacccaagcaatcttgaattgtgtgtggaagacttcttggagcttgctgggatcagtttttgccaagaattctatgtacctagccagaccagtttggagatggtactgaatgataaagcaatatctccccccaaatctctcatatggtctgtgtatccacgtaactaccttccgcaaaacatacaagacagattcaggttggagtctcttgacagactagagcatgagcgcaccaggagagtgctagagttctacttaaaagatgctaattttaagcaaagtgatctcaaaaaatatgttgtaggtcaagagtacttgaatgacaaagagcatgtggtctccttaacagggaaagaaagggaactaagtgtgggtcgtatgtttgcgatgcaacctggaaagcaaagacaggtgcagatccttgctgagaaattgttatcagataacatagtgccatttttcccagagacattgactaggtatggagatctagaattacaaagaatcatggaactcaagtccgaactatcgtctacaaaaaaacggaaaagtgatagctacaataattatatagcaagagcttcaatagtgactgatttgagtaagtttaaccaagcttttagatatgagactacttcagtgtgtgctgatgttgcagatgaactgcatgggactcaaagccttttctgttggttacacctgactgtatcctccacaaccatgatctgcacttatagacatgctccccctgatacaggtggaatctacgacatagaccagataccggagcaaagcggactgtataggttccacatgggtggcatagaaggctggtgccaaaagatgtggaccatggaagcaatatcacttcttgatgtggtgtctgtgaggaatcgggtgcagctcacatcactgctgaatggggacaaccagtctatagatgtgagtaaacctgtgcgactcacaggtgctcaaactgaaattcaggctgattatagccttgccattaagatgttaactgctgtaagggatgcctactataacataggacacaagttgaaggagggagagacttatgtttcaagagacctccagtttatgagcaagaccatccaatcagaaggcgtaatgtatcctgcttcaattaagaaggttctgcgagtgggtccctggataaacacaatactggatgacatcaaaactagtatggaagctattgggagtctctgtcaagagctagaattccgaggggagtcatttgtgactagtcttataattcggaatttctggttgtacatccagtgctttgtcgaggcaaaaaatcatagcctagcaggagcacaaatatccaatgagcttaatagaactttaaccaaagttaagagattcttcaggttagcagatgagagcagcactgttaacttgttcatgaacataccaatgcagttagggggaggggaccctgttgttgtttataggagtttttatagacggacaccagattttttgacagaggtgttaacacacatggaactactactaacagcaatagagatggataggggagtttatatgaacttcttcttaacactgttgaacacatgcaaaaatgatagagccactcttaccacactgatgagagacccccaagctattgggtcagaaaggcaggctaaggtaacaagcgaaattaatagaacagcagtaactagtgtgctcagccttgcaccaaaccccttgtttagtgatagtgctgtgcatttcagtcagaatgaggaagaaataggcacagttatggaggatatatcacctgtctacccacatggactgagagtggtgtatgaagcattcccattccacaaagctgaaaaagttgtaaacatgattgctggaaccaagtccataacaaatatcctccaaagaacttctgctataagtggtgtagatattgacagagcagtccacatgatgttgctcaacttgggcttgttgggtagaatgcttgagtctggacccatcactgacacaatagatctgaggagcaacaatcggattctttgttgtcagctttccaaaagaataagagaaatgtcatgggatgggttggagattgtcggtgtatcatctccaagcatgttgtctagcttagatattaattatgttacaatagcacagaagccaggtatcattgtagaaaaattctcagctgaaaaaacaacaagagggaaaagaggtccgaaagcaccatgggtaggctcgagcacacaagagaaaaaattggtagctgtttacaacagacaggttttatcgaaagaacaaaaagaccaattagaaaccataggcaaaatcaggtgggtctacaaaggcgttactggcctccgacgtcttctaaacttaatatgcttaggaacactgggtcttccatataagctgttaaaaccacttctacctcggtttatgagcgtcaatttcctacacaggcttgcagtgagcagcaggcctatggagttcccatcatcagtaccggcatataggactacaaactttcattttgatactagtcctataaacaagcggttgagtgagaggtttggcaatgaggacatcaaccttgtgttccaaaatgctataagttgtggaattagtgtcatgtgtgtggttgaacaattgaccgggcgtagccctaaacaagtgatactagaacctattgtagaagatattgacataatgtccgcccctaatttccaaggccgcctgaatttcaagagtgtaagaaagattgtgactgatcaacatatctttaatccggaccacattgacttggttatgcttgggaagttgttattaccaactgtccgtagcaatataaataacaacaaactagcaactgaaaatttctttaatggtaacaacatagtagaggcactaacaagctgtcttgcttgccactggtgcactgttttgattctactcactactgagaatagtattttccagaaagagtggggagatggattcataacagaccatgccttcataaacttcacatggttccttatgagtttcaaaacttatctcctatgccattggcaaactgaaaagaacaagacattggggattgtagaggatcccatagacaggctcatcagagtagacaatagtttttggcggatgatgagcaaggtgttcctggaacccaaagtgaaaagaagacttatgctttacgatacgtctgtattgaatgttctgggtagtatcagtttcaaaaactggtttgttagccggttaagaacagtagactacatggagatcccatggattgtcaatgctgagggtgacatagttgagcaaaaacctgtcaatgaatatctgaaaataatggctgcaggtgtccatgtaaaagtcataatgcttagttactccgatatggcacatgctatgacacgcttgctaaggtgcaagaacatgcaagacaatgtgcccaccataaagaaaacagtatctccaactgatgtcacaccctctgtagaccctactcgggccctactgctgttcccgaaagtcatctttagtaagttgaccacttttaatgcaacatgcagtacagctaagcaatctggcagcaatttgacaaaaaactacatgactttaatgccttggcaccacattaatcgttataactttgtccatagctccacagggtgtaaggtaagtgtaaagggatgtataggcaagttgataacaaaacttgaattaagaattatctacttcatcggtgagggagctgggaatctcatgtccagaactgcatgtgaataccctaatctaaagtttgtatacaggagtctgaaagatgccaatgatcatcatcatcccacagagtatgtgcgtgtaatggggagcatcagcagaataattgacctcggtgaagggttggcaatggaagcaactgatgcaactaagagagaacattgggattttatccacagagtgtgcaaagatcctcttctgctaactatctgtgatgctgaactaaaaaacatggaggatgtcctgcatttagtgatacagtggaggaaacatgtactatcttgtaacatatgcacatcatatgggactaatgtgtacatgtttgttaagtatcatgcgcaatctgaggcaaagaagctaccacattttgtccgagtagtttcaatatatatcatgcaagggagtaaactatcaggatcagaatgttacatgttgttaacattaggacaccagaacatggcaccttgctatggagaggttaggtctgcgaaatcaatcatggctttgacgagcacttttaaacacccctgtagactagacaaatctgccattgaggccaacctgaaatcactagcacccgggttgacactgccacttagtgagcctgctatgcaaaagtacttgttggatctttcaagcttaagtggaattaagaacacgggggcaaccattgggggaagcaaagtagtagagaagaagtgggctttatgcaaagtgtcaaatatagtgaggtggttaagtcacgttctgaaaatgcccaaaggtgatttaaattacgattttttcgaggtgatagaaaacacatacccagacatggtgaagctattggacaacttaaccccgtcagagttgaaaaagctcgtcaaaatcaccgggtacatcctgggcacaaagtaataaggcaatatctactcaacacataattatcacctgaaacacaactgacc

SEQ ID NO: 8 - RNA, 13302 nt, Avian metapneumovirus
  source 1..13302: mol_type = genomic RNA
catttaagcaggttttagaaaaacaatgggacaagtcaaaaatgtctcttgaaagcattagactcagtgacttggagtacaaacatgcaatacttgaagactctcagtatacaattagaagggatgttggtgctaccactgcaattacaccttctgaactgcagccgcaagtatccatattatgtggtatggtgttgtttgcaaaacacaccgactatgagcctgcagcagaggtaggcatgcagtacattagtactgctctaggagctgatagaactcaacaaatactgaaaaattccggtagtgaagtacagggtgttatgaccaagattgtaacacttccagcagagggtcctgtcagaaagcgagaagtgctaaacattcatgatgtaggtgttgggtgggctgatgatgttgaaaggactgcaagagaagcaatgggagcgatggttagagaaaaagtgcaactcacaaagaatcaaaagccgtctgccttggatgctcccgttattctattatgcattggtgccctcattttcaccaagttggcctcaaccgttgaagtaggtcttgaaactgctatccgacgtgcctcaagggtattaagcgatgccatatcacggtaccccaggatggatataccaaggattgccaaatcattctttgaattgtttgagaagaaggtgtattacaggaatttatttattgaatacggtaaggcactcggaggtacatccaccggaagcaggatggagagcctgtttgtgaatatttttatgcaagcttatggggcagggcaaacaatgctgcgctggggtgtcattgcacgatcttccaacaatataatgttaggccatgtatctgtacaggctgagttaaggcaagtatctgaggtctatgacctggtgaggaaaatgggacctgagtcaggattactacacttacgccagagtcccaaagcgggtcttttgtcgttgaccaactgtcccaattttgccagtgttgtcctcgggaacgccgccgggcttggtattataggcatgtacaaaggtcgagcccccaaccttgagctgtttgctgctgctgaaagttatgcacgaacattgaaagagaacaacaagatcaacctagcagccttagggctcactgatgatgagagggaagctgcaacatcctacctagggggagatgatgagagatcatctaagtttgagtaattaaaaaatatgggacaagtaacaatgtctttccctgaaggcaaggatattctaatgatgggcagcgaggctgcaaaaatggccgatgcctatcaaagatcactaaggaacacctctgctggaggccgctctattagtggagaacctattaacacaattgctgaaaaggtgccattaccccctttgtgcaaccccaccacaccaaggggatcatgtataaaacccaacaaggctcctgtctccaaagtcaaagaaatagaatctatatatccaaaactcccaactgctccagttgccactgatactgagacttctaccagtactgagtctgcaaagaagtctaaaaaggtcaagtttgacaaccctaaggtaggaaaatatactaaactggaagaagaggcactagagcttttgtcagatcctgaagaggataatgatgaaaagtcatcaatattgacatttgaggaaaaagatacagcatctacctctattgaagcaagattagaagcaatagaagaaaagttgagcatgatattgggcatgctcaaaacattgaacattgcaactgctggcccaaccgccgccagagatggaatacgtgatgcaatgatagggatgagagaagagctgataaacagcattatgacagaggctaaagacaaaattgcagaaatgatgaaagaagaggatacccagagggcgaaaataggagacggcagcgtgaagcttaccgaaaaagctaaagagttgaacaaagtcctagaagaccaaagctcaagtggagagtcagagagtgaagaagagagtggagagtctgactttaatgaggaagagagtgacatatacaatcttgatctgtagttatgaaaaaatgggacaagtcaaaatggagtcctatataattgacacttatcagggggttccctacactgcagccgttcaggttgacctcattgaaaaagactctaaccctgcaacattgactgtatggtttcccctattccagtcaagtacgcctgccccggtgttattagatcaactaaaaactttatcaatcaccacccagtacacagcatctccggaaggccctgtgctccaagtcaatgcagcagcccaaggtgccgccatgtctgcactacctaagaaatttgccgttagtgctgcagtagcacttgatgagtacagcagactggaattcgggacattgactgtgtgtgatgtgagatccatttatctgactaccttgaagccatatggcatggtctccaaaataatgacagatgtgaggtctgttgggaggaagacccatgacttgatcgcattgtgtgattttatagacatagagaaaggggtgcccataaccatacctgcatatattaaagctgtctctatcaaagactcagaatcagccactgtggaagctgcaataagtggagaagcagaccaggctataactcaagcaaggatagctccatatgcagggctgatcttgattatgactatgaacaaccctaaagggatctttaagaaattgggtgcaggtatgcaagtgatagtagaactggggccgtatgtacaagcagagagtctgggaaaaatctgcaagacatggtatcatcaacgaaccaggtatgtactgaggtcacgttgagggtaccctaaagcataaatctgttaatgtcatagtcaataaaaaataaaaaattgggacaagtaggatggatgtaagaatctgcctcctattgttccttatatccaatccgggtagctgcatacaagaaacatataatgaagaatcttgcagtactgtaactagaggctataagagtgtgttaagaacagggtggtatacgaatgtatttaaccttgaaatagggagtgttgagaacatcacttgcaatgatggacccagcctaattgacactgagttggtactcacaaagaatgctttgagggaactcaaaacagtgtcagctgatcaagtggccaaggaaagcagactatcctcacccaggagacgtagatttgtactgggtgcaattgcacttggcgttgcaacggctgctgccgtaacagctggtgtagcacttgcaaagacaattagattagagggagaggtgaaggcaatcaagaatgccctccggaacacaaatgaggcagtatccacattagggaatggtgtgagggtactagcaactgcagtcaatgacctcaaggaatttataagtaaaaaattgactcctgctattaaccggaacaaatgcaatatagcagatataaagatggcaattagttttggccaaaataacagaaggttcttgaatgtggtgaggcaattctctgatagtgcaggtatcacatcagctgtgtctcttgatttaatgacagatgatgaacttgttagagcaattaacaaaatgccaacttcatcaggacagattagtttgatgttgaacaatcgttcaatggttagaaggaaggggttcggtatattgattggtgtttatgatggaacggtcgtttatatggtacaactgcccatattcggcgtgattgagacaccttgttggagggtggtggcagcaccactctgtagggaagagaaaggcaattatgcttgtatactaagagaagatcaagggtggtactgtacaaatgctggctctacagcttattaccctaataaagatgattgtgaagtaagggatgactatgtgttttgtgacacagcagctggcattaatgtagcctcagaagttgagcagtgcaactataacatatcgacttctaaatacccatgcaaagtcagcacaggtaggcaccccgtcagtatggtagctttaacccccctagggggtctagtgtcttgttatgagggtgtaagttgctccataggcagcaataaagtagggataataaaacagctaggcaaagggtgcacccacattcccaacaacgaagctgacacaataaccattgataacactgtgtaccaattgagcaaggttgtaggtgaacagaggaccataaaaggagccccagttgtgaacaattttaatccaatattattccctgaggatcagttcaatgttgcacttgaccaagtatttgagagtatagatagatctcaagacttaatagataagtctaacgacttgctaggtgcagatgccaagagcaaggctggaattgctatagcaatagtagtgctagtcattctaggaatcttctttgtgcttgcagtgatatattactgtttcagagtccagaagaccaaaccaaagcatgattatccggccacgacaggtcatagcagcatggcttatgtcagttaagttatttaaaattgggacaagtgaggatgtctaggcgaaatccctgcagatatgaaatacggggcaaatgcaacaggggatcatcctgcacgtttaaccataattattggtcctggcctgatcatgtgctcttagtcagagcaaactacatgcttaaccaattagtgaggaacacagacaggacagatggcctatccttgatatcaggggcaggacgagaagatagaacccaggattttgttctaggatctgccaatgttgtacagaattacatagaagggaacacaacaatcaccaaatcatcagcctgttacagcctgtacaacattataaaacagctccaggaaaatgatgtgaagacatccagagattcaatgcttgaagaccctaaacatgtagcgctacacaacctgatactatcttatgttgatatgagtaaaaatccggcaaatttaatcaacagcctcaagcggcttcctagagaaaaactcaaaaagcttgccaagattatattacagctatctgcagggtcagagagtgacaatgccagtggtaataccctgcaaaagggtgacagcaataattaagtgcaatgcacttggattgtgtatggttcgtaagatatatgattatagtattgcaagctggagtgatttaatagaggaagtagccaatatgatcctaatagatcacattaataggaaacaatgtgtggagtgtcgaaaagattttgagtttatagctatatatacctcatataattagttaattaaaaccaattaagctataagtccaaagaagggtgggacaagtcataatgacttctaccgtcaatcttgggtcagacactgcttcaaaatggaaagtgatcaagtcaaggtgcaattcctgctgcaggatcttggtgagttgtgctgctgtgatttgtgcaatcttagctttgatcttccttgttgctacgatcggattgtctgtgaaactggcattcacagtgcaggagatccacaactgcaagcagaagctatcaggggcgagcacaactacagctgcaatctatactactccaagcacaatgatagaagcactacagacaaatcaacttaaactgacaaccaatgaaggacgtagtactctccctgattgcctggtggagaaaaaattgtgcgaaggtgaggtcaggtatctcaaaactaaaggctgcttgggggcacgagagggtgaagatttgaattgtatagatttagtagtggagtgtgttgggaaaccctgtggtcacaacgaggattacaaagaatgcatatgcaccaacaatgggacagcaactaagtgctgctacatctaatgaatattgacaacactagtgccaaataataggcaacagcattatttaattaaaaagaagaaagatcgggacaagtatctcaatggggtccaaactttatatggttcagggcaccagtgcatatcaaactgcagtggggttctggctaaatattgggaggaggtacatattggctatagtcctatcagctttcgggctgacctgcacagtcactattgcactcactgttagtgtcatagttgaacagtcagtgctagaagagtgcagaaactacaatggaggagatagagattggtggtcaaccacccagaagcagtcaactactgcaccaagtgcgactccaacaggaaattatggaggattacaaacggctcgaacaagaaagtctggaagctgtttgcatgtgcaaatttcttatggtgatatgtataaccgcagtaatactgtactgggtggttttgattgtatgggcttattggctctttgcaaatcaggaccgatttgtcagcgagataatcaagttgacccaacagtcctctgccactgcagggtaggtctttcaagtgtggactgctgcaaggtgaacaagatcagcaccaacagcaacaccacctctgagctccagaagaccaacccgacatggcatagccaagacaacacagactccaatccaaatccccaaagcataactaccagcacagccactctgctctcaacaggtctgggcctcatgctcacatcgaagaccgggacacacaaatcagggccctcccaagccctgccggggagcaacaccaacagaaaaacaaccacggaccgagaactagggtccacaaatcaaccaaattcaaccaataatgtgcaacatgataagcacacccaacgaacgacacccctgccaggctacaacaacacaagaactatcctccagcacacaacactctgggagaagacactcagcacatacaagcccacacactctttaaccaacgaatcagatcaacccctctccacaactcaaaacagcatcaactgtgaacattttaacccccaaggcagagaaaaaatctgctacagagtaggttcttacaactccaatattacaaagcaatgcagaattgatgtgcctttgtgttccacttataacacagtgtgcatgaaaacatactataccgaaccattcaactgttggaggcgtatctggcgctgcttgtgtgatgacggagttggactggttgagtggtgttgcactagttaactaaaaatgaaaacacatacatccgataatagaaaaatacatcagatagtctaaaacaattaaaagaatgaaaacaaggaccaatatggaaatatccgatgagtccgtcgtaaatgtctatctgcctgactcttacctaaagggggtcatttcattcagtgagaccaatgcaattggatcatgtgtactcaaccggccatacattaaagatgaccatacagcacatgttgcaatgactaacccagtgattgaacaccagaggttgagggccctcttcaagagtcttactattagcagagaatatcgagtggttgaacctcttatgatccagaaggagctgctaaaagtcgcagcaggtgcaagactaaagaaactcaagaaatggttgggtagaagcaaggatataagtgaagtcaaattgaaaatggtgactgattggcttaaactgtcacagacaccaggaaggggaaaaataatagattgtattcaagtggagaacttacctgactggctggagcactggtttgattcttggttgatattgaatgacgtcatccaatcttataggtgtttggaagtatcccaaaccagtgctatattacggaaaagtagcctgaacttcttttttgctgtttcttcttttgggtgtataatcataagtcgcaagagtagaagaatatgtttttgcacctacaatcaactcctgacctggaaggaccttgctttaagcagattcaatgcaaatttatgtgtgtgggtgagcaattgcttaaatagcgcacaagacggcctcggactaaggagcaaattagttggtgagttactcaaccgattgtactgtgagactgatgagttattgagcatcactggcaatgaggggtatgggattgtaaaggaatttgaaggattcattatgagtgaaatattgcggatgactgaacatgcacaattcagtgtaaggttccggaatacgctattgaatggcatagctgataaaatagggaagatgagagccgcataccaaagacggacacagaacacatctgttgagcatcatagatacccaagtgagcatgaaatgctggaagcaacagggagagtactgagaataattaagcttctggtgaataatgagattgctaatgctgcagagatgtattttatattccgcatattcggacatcctatggtagaagaaagggaggctatggatgcagtgagggataacagtgaaactgcaaaaatcttaagtctggtggcccttacggagatgaggggggccttcattctaagagtgatcaaaggtttcgtagggaactacaaaagatggccaaagataaagaatccatcaacattgagtcgaaggtggaagatgtacatgaatgcaaaaacatacccaagcaatcttgaattgtgtgtggaagacctcttggagcttgctgggatcagtttttgccaagaattctatgtacctagccagaccagtttggagatggtactgaatgataaagcaatatctccccccaaatctctcatatggtctgtgtatccacgtaactaccttccgcaaaacatacaagacagattcaggttggagtctcttgacagactagagcatgagcgcaccaggagagtgctagagttctacttaaaagatgctaattttaagcaaagtgatctcaaaaaatatgttgtaggtcaagagtacttgaatgacaaagagcatgtggtctccttaacagggaaagaaagggaactaagtgtgggtcgtatgtttgcgatgcaacctggaaagcaaagacaggtgcagatccttgctgagaaattgttatcagataacatagtgccatttttcccagagacattgactaggtatggagatctagaattacaaagaatcatggaactcaagtccgaactatcgtctacaaaaaaacggaaaagtgatagctacaataattatatagcaagagcttcaatagtgactgatttgagtaagtttaaccaagcttttagatatgagactacttcagtgtgtgctgatgttgcagatgaactgcatgggactcaaagccttttctgttggttacacctgactgtatcctccacaaccatgatctgcacttatagacatgctccccctgatacaggtggaatctacgacatagaccagataccggagcaaagcggactgtataggttccacatgggtggcatagaaggctggtgccaaaagatgtggaccatggaagcaatatcacttcttgatgtggtgtctgtgaggaatcgggtgcagctcacatcactgctgaatggggacaaccagtctatagatgtgagtaaacctgtgcgactcacaggtgctcaaactgaaattcaggctgattatagccttgccattaagatgttaactgctgtaagggatgcctactataacataggacacaagttgaaggagggagagacttatgtttcaagagacctccagtttatgagcaagaccatccaatcagaaggcgtaatgtatcctgcttcaattaagaaggttctgcgagtgggtccctggataaacacaatactggatgacatcaaaactagtatggaagctattgggagtctctgtcaagagctagaattccgaggggagtcatttgtgactagtcttataattcggaatttctggttgtacatccagtgctttgtcgaggcaaaaaatcatagcctagcaggagcacaaatatccaatgagcttaatagaactttaaccaaagttaagagattcttcaggttagcagatgagagcagcactgttaacttgttcatgaacataccaatgcagttagggggaggggaccctgttgttgtttataggagtttttatagacggacaccagattttttgacagaggtgttaacacacatggaactactactaacagcaatagagatggataggggagtttatatgaacttcttcttaacactgttgaacacatgcaaaaatgatagagccactcttaccacactgatgagagacccccaagctattgggtcagaaaggcaggctaaggtaacaagcgaaattaatagaacagcagtaactagtgtgctcagccttgcaccaaaccccttgtttagtgatagtgctgtgcatttcagtcagaatgaggaagaaataggcacagttatggaggatatatcacctgtctacccacatggactgagagtggtgtatgaagcattcccattccacaaagctgaaaaagttgtaaacatgattgctggaaccaagtccataacaaatatcctccaaagaacttctgctataagtggtgtagatattgacagagcagtccacatgatgttgctcaacttgggcttgttgggtagaatgcttgagtctggacccatcactgacacaatagatctgaggagcaacaatcggattctttgttgtcagctttccaaaagaataagagaaatgtcatgggatgggttggagattgtcggtgtatcatctccaagcatgttgtctagcttagatattaattatgttacaatagcacagaagccaggtatcattgtagaaaaattctcagctgaaaaaacaacaagagggaaaagaggtccgaaagcaccatgggtaggctcgagcacacaagagaaaaaattggtagctgtttacaacagacaggttttatcgaaagaacaaaaagaccaattagaaaccataggcaaaatcaggtgggtctacaaaggcgttactggcctccgacgtcttctaaacttaatatgcttaggaacattgggtcttccatataagctgttaaaaccacttctacctcggtttatgagcgtcaatttcctacacaggcttgcagtgagcagcaggcctatggagttcccatcatcagtaccggcatataggactacaaactttcattttgatactagtcctataaacaagcggttgagtgagaggtttggcaatgaggacatcaaccttgtgttccaaaatgctataagttgtggaattagtgtcatgtgtgtggttgaacaattgaccgggcgtagccctaaacaagtgatactagaacctattgtagaagatattgacataatgtccgcccctaatttccaaggccgcctgaatttcaagagtgtaagaaagattgtgactgatcaacatatctttaatccggaccacattgacttggttatgcttgggaagttgttattaccaactgtccgtagcaatataaataacaacaaactagcaactgaaaatttctttaatggtaacaacatagtagaggcactaacaagctgtcttgcttgccactggtgcactgttttgattctactcactactgagaatagtattttccagaaagagtggggagatggattcataacagaccatgccttcataaacttcacatggttccttatgagtttcaaaacttatctcctatgccattggcaaactgaaaagaacaagacattggggattgtagaggatcccatagacaggctcatcagagtagacaatagtttttggcggatgatgagcaaggtgttcctggaacccaaagtgaaaagaagacttatgctttacgatacgtctgtattgaatgttctgggtagtatcagtttcaaaaactggtttgttagccggttaagaacagtagactacatggagatcccatggattgtcaatgctgagggtgacatagttgagcaaaaacctgtcaatgaatatctgaaaataatggctgcaggtgtccatgtaaaagtcataatgcttagttactccgatatggcacatgctatgacacgcttgctaaggtgcaagaacatgcaagacaatgtgcccaccataaagaaaacagtatctccaactgatgtcacaccctctgtagaccctactcgggccctactgctgttcccgaaagtcatctttagtaagttgaccacttttaatgcaacatgcagtacagctaagcaatctggcagcaatttgacaaaaaactacatgactttaatgccttggcaccacattaatcgttataactttgtccatagctccacagggtgtaaggtaagtgtaaagggatgtataggcaagttgataacaaaacttgaattaagaattatctacttcatcggtgagggagctgggaatctcatgtccagaactgcatgtgaataccctaatctaaagtttgtatacaggagtctgaaagatgccaatgatcatcatcatcccacagagtatgtgcgtgtaatggggagcatcagcagaataattgacctcggtgaagggttggcaatggaagcaactgatgcaactaagagagaacattgggattttatccacagagtgtgcaaagatcctcttctgctaactatctgtgatgctgaactaaaaaacatggaggatgtcctgcatttagtgatacagtggaggaaacatgtactatcttgtaacatatgcacatcatatgggactaatgtgtacatgtttgttaagtatcatgcgcaatctgaggcaaagaagctaccacattttgtccgagtagtttcaatatatatcatgcaagggagtaaactatcaggatcagaatgttacatgttgttaacattaggacaccagaacatggcaccttgctatggagaggttaggtctgcgaaatcaatcatggctttgacgagcacttttaaacacccctgtagactagacaaatctgccattgaggccaacctgaaatcactagcacccgggttgacactgccacttagtgagcctgctatgcaaaagtacttgttggatctttcaagcttaagtggaattaagaacacgggggcaaccattgggggaagcaaagtagtagagaagaagtgggctttatgcaaagtgtcaaatatagtgaggtggttaagtcacgttctgaaaatgcccaaaggtgatttaaattacgattttttcgaggtgatagaaaacacatacccagacatggtgaagctattggacaacttaaccccgtcagagttgaaaaagctcgtcaaaatcaccgggtacatcctgggcacaaagtaataaggcaatatctactcaacacataattatcacctgaaacacaactgacc

SEQ ID NO: 9 - RNA, 13472 nt, Avian metapneumovirus
  source 1..13472: mol_type = genomic RNA
tcaagtcacaatagaaaaggaaaatgggacaagtaaaaatgtctcttgaaagtattagactcagtgatctagagtacaagcatgcaatccttgatgagtcacaatatacaataaggagagatgttggtgctacaactgctatcacaccctctgaactacagccaaaggtatccaccctgtgtggaatgattttatttgctaagcatgcagattatgaaccagcagcagaagttggcatgcagtacatcagcactgcattaggagctgataagacacagcagatactcaaaagctctggcagtgaagtgcaaggggtcatgaccaagatagtgacactccctgcagaaggtcccatacgaaagcgggaagtacttaatatccatgacattgggcccgcatgggcagacaatgtagaacggactgcaagggagaccatgagcctaatggtaaaagagaaagctcaaatacccaaaaaccaaaagccgtcagctcttgatgcacctgtgatcttactatgtattggggcattaatattcactaagcttgcatccacagtagaggttgggctggagactgctattaggagagcctctcgggtgttaagtgatgcaatatcacgttaccctaggatggacataccacggattgccaaatccttctttgagctatttgaaaagaaagtgtactataggaaccttttcatagagtacggtaaggcacttgggagtacgtcttccgggagtaggatggagagcctctttgttaacatcttcatgcaagcttatggagctgggcagactatgctaagatggggtgttgtggcaagatcatccaataacatcatgttgggccatgtgtctgtgcaggcagagttaaggcaggtctcagaagtgtacgatcttgttaggaaaatgggtcctgaatcaggcctcctccacttgaggcaaagtccaaaagcaggcttactatcattaacaagttgccccaactttgcaagtgttgttttggggaatgcagctggcctaggcatcattgggatgtataagggcagagcacccaacttggagttgttttctgcagcagaaagttatgctagatccctcaaagagagcaacaagattaaccttgctgcccttgggctaactgaagatgagagagaggctgccacatcatacctgggcggagatgaagacaagtcacagaagtttgagtaattaaaaattcgggacaagtgaaaatgtctttccccgaaggcaaggatatcttgatgatgggaagtgaggcagctaagttggcagaggcttatcagcaatcaatcaagaattccacttctgtgagaagatccattagtggtgaccctgttagcacagtgtctgaaaaagttccattaccaccactatgtagctcagaaacttctagaggagcatgcataagacccacaaaatcaaccctgcctcccatcaaggaagtggagtccatctacccaaaactacccacagctccccctgatgccatgattgagaccgcccaccccgtaggggctccaaagaaggcacagaaaagggtgaaatttgagagttcaaaagcaggcaaatacaccaagctagaagaagaagccctggagctcctgtccgaccctgatgaagataatgatgagaaatcatctgtacttacatttgaggagaaggacaatgcaccatcatctatagaggcgaggttggaggctatagaggaaaaactcagcatgatattggggatgttaaagaccctgagcatagccaccgcagggccaaccgcagcgagagacggaatacgtgatgcaatggtgggagttagagaagagttgatcaatagcattatggcggaagctaaagggaagattgcagaaatgataaaggaagaagatgcacagagggcaaaaataggagatgggagtgtgaaactaacagagaaagctcgggaattaaacaggatgcttgaggatcaaagctcgagtggtgaatctgagacagaaagtgaagagactgaaccagatactgatggtgaaaatgatgatatatacagctttgatatgtagttatgaaaaaatgggacaagtaaacatggagtcctatattatagatacctatcaaggtgtgccctacacagctgctgttcaagtcgatctagttgagaaggacaacaaccctgcaaaactgacggtttggtttcctttatttcagtccagcactcctgctccagtgctgctggaccagctaaaaactctgtcaatcactacacaatatactgcttcccctgaaggaccagtgctacaagtaaatgctactgcacaaggtgcagccatgtcagctctgcccaagaagttttcagttagtgctgctgtagcacttgatgagtacagcaaacttgactttggggtactcacagtttgcgatgtaagggcagtttacctcaccactctgaagccatacggtatggtgtctaagattgtaacaaacatgaacaccgttgggcgcaaaactcacgacctgatagccctttgtgacttcattgacatggagagaggaataccagtaactatccctgcctacataaaggcggtatccatcaaagattctgagtcagctacagttgaagctgcgatcagtggtgaagctgaccaggctatcacccaggcaagaatagctccgtatgcagggttgattctggtcatgaccatgaacaaccccaaagggatattcaggaaactcggtgcaggaacacaagttattgtggagttagggccttatgtgcaggctgagagcttagggaaaatatgtaaaacatggaaccaccagagaaccaggtatgtcctgaagtcccgctaaaactggcaaacaaaagtctactattcttctgtagtttaataaaaatatggggcaagtaaaatgtacctcaaactgctactaataatttatttggtggttggggccagtgggaagatacaagaaacttacagtgaagaatcatgcagcactgtaaccaggggttacaaaagtgtgctcagaacgggttggtatacaaatgtgttcaacctagaaatagggaatgtggagaacataacatgtaatgatggtcctagccttatcagcactgaattatcactaactcagaatgccttgcaagagcttagaactgtttctgccgatcagattacaaaggagaatcgaatcctttcccataggaagaagaggtttgtgttgggtgcaattgcccttggagtggccaccacagctgctgtaacagccggcgtagctttagctaaaacaataagattagaaggggaggtgaaagccatcaagctagctttgcgcagtacaaatgaggctgtgtccacattaggcaatggcgttcgcatcttagcaacagctgttaatgacctaaaagaatttataagcaagaaattaacccctgcaataaaccaaaacaaatgcaacatagcagacataagaatggcaatcagcttcggacagaacaacagaaggtttctaaatgtggtgagacaattttctgacagtgcaggaataacttccgcagtgtctttagatcttatgacagatgcagaattggttaaagccatcaaccgaatgccaacttcgtctggccagattagcctcatgctgaacaatagagcaatggttaggaggaaagggtttggaatacttataggtgtttacggggggactgtagtgtatatggtgcaactcccaatatttggagttatagaaacaccctgctggagagtggtagctgcacccctctgtagacatgagagggagagttatgcttgtctgctgcgggaagatcaggggtggtactgcactaatgcaggatcaactgcttactacccaaatgaagatgactgtgaggtaagagatgactatgtgttctgtgatactgcagcaggtataaatgttgcatcagaggtggagcagtgcaaccaaaacatatcaacctctacttacccttgcaaagttagcacagggagacaccctgtaagcatggtagccttaacccctttgggaggtttagtatcatgttatgaaggtgtgagttgctccattggcagcaacaaagtcggaatcattaaacaactcaacaaaggatgcacacacatacccaataatgaggcagacacaataactatagataacacaatttatcaactcagcaaggtggtgggtgagcaaaggaccatcaaaggagctccggtggtaaataacttcaaccctctactgttccctgaggatcaatttaacgtagccttagatcaagtatttgagagtgttgataaatcaaaagacctgattgacaagtctaatgacttactggacatagaagttaagagtaatataggtgctgcattagccatcacaattttagtagtgcttagcatgtcaatcatagtgggcatagcttactatgtggttaaaaagaggaaagccaaagcacccaatggatatcctaaaacaacagggcaaagcaacatgggctacatcagttgagttacataaaaaatgggacaagtaaagatgtccagaaggaatccctgcagatatgaaacaagaggcaagtgcaaccgagggtcctcatgtacatttaaccacaattactggtcttggccagaccatgtattgttagtgcgggctaattatatgctgaatcagctagttaggaacacagataggactgacgggttatctctcatatcaggagcagggagagaagataggacacaggattttgtgctcggttctgccaatgttgtccagaattatatagaagggaatgcaaccataacaaaatctgcagcttgttatagcttgtacaacattatcaagcaacttcaagagaatgatgtgaaatctgcacgagacctgatggtagatgaccccaagcatgttgccctgcataaccttgtcttgtcctatatagacatgagtaagaatccagcaaatctcataaacagtctcaagagactacctaaagaaaaacttaaaaaattggccaaaatcataattcagctttctgctggttcagaaggagaaaatgccaatagtaataccctgcaaaagggtgacagcagtaattaagtgcaacacctcaggtgtgtgcctgtttaagaggacatatgagcataacatcattagccttggtgatctgatagaagatgtagcgaggatgattattatagatcacataaatagaaaacaatgcaatgaatgtagaaaagattttgaatttgtagcggtttacacatcttacacttagttatataaaaacaattgagcagctacccccaaagaaagatggggcaagtcaagatgacctccactgtcaaccttggatcaagcacatcctcaagatggactatagcaaagtcgcggtgcatgctgtgtcttcggactatgatgaattgtgctgttgttatatgtgcagtgctagttttgatcttccttgttgctacgataggcttgtctgtgaagttggctgtcacaataaaagaaaggaatacttgccagctgaggctatcagagctgagcacaactacagcatcaatcctgagatcaacaaatcagccatatccaggagggtcaacaagcacacccaaactgacaactgtcacatctattaccgatctcacccaccagtgtcctcaaaggaaagagttatgcaatggaacaataacatacaaaaactctgatggatgcctggatgaaaaagaaggagaatccattgactgtatagagctcatagccagatgtgtggaaactctatgcgaccccaaccctaactacaaccactgtatgtgcaccaagaacagcactgggctctggtgttgttacaattagagaaggggtaaacccatcatgcactcaaccactgcaaccagcatatgaatatatacatacaagtagattagttaattaaaatcagaagacgggacaagtatccagatggggtcagagctctacaccatagagggggtgagctcatctgaaatagtcctcaagcaagtcctcagaaggagcaaaagaatactgttaggactggtgttatcagccttaggcttgacgctcactagcactattgttatatctatttgtattagtgtagaacaggtcaaattacgacagtgtgtggacacttattgggcggaaaatggatccttacatccaggacagtcaacagaaaatactccaacaagagataagactgcaacaaaagaccctagaagattacaggcgactggagcaggaaagtttgagagctgtgggtatgtgcaagttgttgatggtgatatgcatgatcacagttatgctgtactgggtggtgttgattgtttgggcttattggctctttgtgaatcaggaccaatttgtcagggagatacttggtctgaaggcggaaacttctgccgatgcactttttcttcccatggggtgagttgctgcaaaaaacccaaaagcgaggcaaccactgcccagaggaaccccaaaccagccaacagcaaatcaactcctccggtacattcagacagggccagcaaagaacataattcctcccaaggggaacaaccccgaagggagccaaccagcagcaatacaactattgctggcaccccatcaacagaggacactgctaagccaacgatcagcaaacctaaactcaccatcaggccctcgaaaagaggttcatccagcagcacaaaggcagcctccagaacccccaaccacaagaccaacaccagaggcaccagcaagacgaccgaccagagactccgcaccggacccactcctgaaaggcccagacaaacccacagcacagcaaccccaccccccacaaccccaatccacaagggccgggccccaacccccaaaccaacaacagacctcaaggtcaacccaagggaaggcggcataagcccaactgcaatacagaaaaacccaaccacacaaagtaatctcgtcgactgcacactgtctgatccaaatgagccaccaaggatttgttaccaggtaggaacttacaatcctagtcaatcaggaacctgcaacatagaggttccaaaatgttccacttatgggtatgcttgtatggctacattatatgacaccccattcaactgctggcgcagaaccaggagatgcatctgtgattccggaggggagctgattgagtggtgctgtactagtcaataaaaagtaacctttcacatctaaaataaagcaaaaagaagaaaaaagaagaaagaaggaaagaaagaagaagaatagcacacaacagaaaggagggaccaatatggacccatccaatgagcacacggtgaatgtgtatctgcctgactcctatttaaaaggagtcatctctttcagcgaaacaaatgcaattggatcttgcattataggtcgaccatacctaaaggatgactacactgctcatgttgcaaccacaagtcctgtcattgagcaccaaagattaaaaagcttgtttagtggtctaacaatcagcaagaactatcaatttgttgaacccctcatgatgcagaatgagctgctcaagatctctgccggtacaaaactcaagaagctaaagaaatggctaggtcgaagcaaagatattagtgagatcaagctgaagatggttgcagactggctcaaacttgcacatactcctgggaaagggaaagtgattgactgtatccaggtagacaacctacctgaatggctagagcaatggtttgattcatggttgatgcttaatgaactgatacaagcttataggtgcctggaagtgtcacagaccagtgctatcctccgtaaaagctgttggaatttcttctttgctatatcatcatttggctgcatcttgataagccggaaaagtaaaaggatttgtttctgcacctataaccagttcttgacatggaaagatttggcacttagcaggttcaatgctaacctgtgtgtctgggttagcaactgtttaaatagtactcaagaaggactaggactccggagcaaacttgttggagaactcctaaatcgattatatcatgagactgatgagttgttgagtggtactggcaatgagggattctggatagttaaggaattcgagggattcatcatgagtgaaattctgaggataactgaacatgcacagtttagtgtacgctttagaaacacactattaaatggaatcgtagaaaaaattgggaaaatgcgtgttgcccacagtcagcgaacttccaacactgccatagaaaatcatagatatcccggagagcaaagtttgttggagaaaacaggaagagtgctcaagattataaagctactagtccaaaacgatatgtccaatgcagcagagatgtattttatctttagaatattcggtcaccctatggtagaagaaagggaggccatggatgctgttcgggagaacagtgaggccactaaaatattgagcctcagggctctgacagagatgagaggagcttttatcctaagagtgatcaaaggttttgtaacaaattacaagaggtggccaagaataaagaacccttctacattgagtagcaggtggcgaatgtatatgagggctaaaacatatcctagtcaacttgagctatgtgctgaagacttcttagagttagcaggtatcagcttctgccaagaattctatgttcccaacaggaccagcctagaaatggtactcaatgacaaggcaatctcccctccaaagtctttgatctggtctgtatatccaaaaaattatttacctccttctgtacaggagcaatttcggttagaatctctggggaaagcagaacatgaaaaaactagacgtgtactagagttttacttgaaggatgccaactttaaccaagagaacttgaaaaagtatgtagtgttacagcactaccttaatgataaagagcatgttgtctctttgactggaaaagaaagggaattaagtgtggggaggatgtttgccatgcagccaggaaaacaaagacaagtgcagatacttgcagaaaaactgttatctgataatatagtaccatttttcccagaaactcttacaagatatggagacctggagttacaaagaattatggagcttaaatctgaactatcttcagtgaaagctaggaagagtgatagctacaacaattatatagcccgggcatcaatagtaacagatcttagcaagttcaaccaagcatttcggtatgaaacaacatcagtttgtgctgatgttgccgacgagctccatggtactcagagcctattttgctggttacatctaacagtttccgccaccactatgatatgcacttataggcatgcaccacctgacaccaaagggatttatgatattgactcaatccctgagcagagtgggttgtatagataccacatgggaggaattgaggggtggtgtcaaaaaatgtggaccatggaggcaatatctctccttgatgttgtgtcggttaagaacagggttcagctaacatcactcctaaatggtgacaatcagtccatagatgttagtaaacctgtgaggcttattggtactcagactgagatacaagcagactacagtttagcaattaaaatgttgactgcagtcagggatgcttattctgacataggacacaaattaaaagaaggtgaaacttatgtgtcaagggatctgcaattcatgagtaagactatacagtctgaaggggtcatgtacccagcctcaattaagaaagttctgagagttggcccatggataaacactatactagacgatatcaagacaagcatggaagccataggtagcctttgtcaggagttggagttccggggggaatcatttactacaagcttgataatacggaatttttggttatatgtccaatgttttgtagaagcaaagagacacagtttagcaggtagtcagatatctaatgaattaaatagaacacttaccaaagtaatgaaatttttccaagtaggcagtgagtcagacacagtaaatctttatatgaatataccgatgcagctgggaggcggagaccctgttgttgtgtataggagcttctataggcggacacctgacttcctaacggaagtactaactcatgtagagttgttactgacatcacttaatgttgatagaggtgtttacatgaggttcttcctcacactaatgaacacctgcaaaaacgacaatgcaacattaactacactcatgagggatcctcaggctattgggtcagaaaggcaggctaaaataacaagtgagatcaatcgtacagcagtcacaagtgtactaagcctggctcctaaccagctttttagtgatagtgcagtacattttagccagaatgaggaagaaattggtactgttatgcaagatgtagggcctgtataccctcatggcttaagggtcatatatgaagcattcccattccacaaagctgaaaaagtagttaacatgatagcaggtaccaaatctattacaaatatattgcaaaggacatccgccataagtggtttagacattgatagagcagttcatatgatgttgctcaatctaggactgttagggaggatattagagtcaggacctgtcacagacaccattgagttgcgtcacaacaataggatactttgttgtcaattatctaagcgcatacgggaaacatcatgggacggaatagaaattgtaggagtgtcatcccctagtatgttatcatgcttggatataaactatgtaacagcagcacagaggccaggagtattaatagagaagttctcggcagaaaagactacaagagggaagagagggcctaaggctccatgggttggatctagcacgcaggagaaaaaattaacagcagtgtacaataggcaagccttatcaaaagagcagagagatcagttagaaacaataggtaagattagatgggtgtacagaggggtaacaggactacggagacttttagatctagtctgcatgggaaccttaggacttccctacaagctgataaaacccctgttaccaagattcatgagtgtcaatttcctgcatcgtcttgcagtgagcagtagaccgatggagtttccttcatcagtcccagcataccgcacaactaatttccactttgacacaagccccatcaataaaagacttagtgagagatttggaaatgaggacattaacttggtcttccaaaatgctataagctgtgggattagtgtcatgtgtatagtggagcaattaacagggaagagcccaaagttagtcgtgctagagccaattgtggaagatatagacataatgtctgctcctaatttccaagggagggtaaatttcaagagtgtgaagaaaattgtggctgatcagcacatcttcaaccctgatcatatcagcctcacaatgctggggagactcctactgcccactataagaagcaactcaaatgcagagagaattgcaactgagaatttctttaatgggaacaatatagtagaggtgctatccagttgccttgcatgccattggtgtacaattttgttgctcctcaccatggagaatagtattttccagaaagagtggggtgatgggtttgttacagaccatgcctttataaactttacatggttcctgatgagcttcaaaacatatttgctctgccactgggggggtgacgatgaaggagagctaaacattgtagaagacccaattgacaggctagctagaatagacaatagtttttggcgtatgatgagcaaggttttccttgaaccaaaagtcaagagacgattgatgctgtatgacaccacaatcctaaatgtctttggcagtatcagttttaaaaactggttcattgggaagcttaggtcagcagattatacagaaataccttggatagtaaatgcagaaggagacattgtggaacagaggcccgtcacggagtaccttaaaaccatggcagctgggactaatgtcaaagtgataatgctgagttactcagacatggcacatgctatgacacggctgcttagatgcaaaaacatgcaggacaatgttccgaccattaagaaggcagctaccccaaccgatgttacaccagctgtggacccaacaagagctttgttgttgtaccctaaagttacctttagcaaactgacaactttcaatgcggcatgtactagcctacgacaacccgggggaaacttgtccaaaaattacataactttgctgccttggcaccatgtgaaccgatacaatttcgtgcatagttctacagggtgcaaggtcagcataagaagctgtctcggtaaattggtggcaaaactagacctcagagttatttactttattggtgaaggtgcagggaacttgatgtcaaggacagcatgtgaatatcctggcttaaaatttgtctacaggagcctaaaagatgctaacgatcatcaccaccccacagaatatgtgcgtgtcatggggagcatcagcagaatagtggacttaggtgaggggttagctatggaagcgactgatgccacgaggagagagcattgggatttcatacacagagtgagcaaagaacccttgttactgacagtgtgtgatgctgaactcaaagaccctaataacgtacttttattaatcatacagtggagaaagcatgtgctttcgtgtaaaatatgcacatcatacgggacaagtgtgtacatgctgatcaaatatcatgcccagcaagagccaaaaaaactcccacattttgtacgggtggtttctatatacgttatgcagggtagtaaagtctccggttcagagtgttatctgctcattactctcggacatcagaacacagccccttgctatggagaggtgcgtgctgcaaagtcagtaatggctctcactgaagtctacaagaatcctataaggctagacaaaatagctgtcgaaatcaatctaaagtctttagccccaggattatctttaccaatcacagaggtgagcatgttgaagtacctttcaaacctaacacacttggacaactccaaaaatattagtgccaccattgggggcagcagagttgtagaaaagaaatgggcaatgtgcaaagttgcaaatgttgtaagatggctatgtcatgtcagtaggatgcccaaaggcgacctgaattatgatttctttgaagtaattgagagcacctatccggatatggtgaaattgctagacaaccttaactcttcagagttgaagaaactagtgaaagtgacagggtacatactgagcacaaaatagaccataaagaatgatcacacagtcgtacacagcaggtaagttgtaactaaaaatccaatagggaaaaggggtgcattagttataaaaaacaaaaaacaaaaattaaaaatgtattg

SEQ ID NO: 10 - RNA, 13472 nt, Avian metapneumovirus
  source 1..13472: mol_type = genomic RNA
tcaagtcacaatagaaaaggaaaatgggacaagtaaaaatgtctcttgaaagtattagactcagtgatctagagtacaagcatgcaatccttgatgagtcacaatatacaataaggagagatgttggtgctacaactgctatcacaccctctgaactacagccaaaggtatccaccctgtgtggaatgattttatttgctaagcatgcagattatgaaccagcagcagaagttggcatgcagtacatcagcactgcattaggagctgataagacacagcagatactcaaaagctctggcagtgaagtgcaaggggtcatgaccaagatagtgacactccctgcagaaggtcccatacgaaagcgggaagtacttaatatccatgacattgggcccgcatgggcagacaatgtagaacggactgcaagggagaccatgagcctaatggtaaaagagaaagctcaaatacccaaaaaccaaaagccgtcagctcttgatgcacctgtgatcttactatgtattggggcattaatattcactaagcttgcatccacagtagaggttgggctggagactgctattaggagagcctctcgggtgttaagtgatgcaatatcacgttaccctaggatggacataccacggattgccaaatccttctttgagctatttgaaaagaaagtgtactataggaaccttttcatagagtacggtaaggcacttgggagtacgtcttccgggagtaggatggagagcctctttgttaacatcttcatgcaagcttatggagctgggcagactatgctaagatggggtgttgtggcaagatcatccaataacatcatgttgggccatgtgtctgtgcaggcagagttaaggcaggtctcagaagtgtacgatcttgttaggaaaatgggtcctgaatcaggcctcctccacttgaggcaaagtccaaaagcaggcttactatcattaacaagttgccccaactttgcaagtgttgttttggggaatgcagctggcctaggcatcattgggatgtataagggcagagcacccaacttggagttgttttctgcagcagaaagttatgctagatccctcaaagagagcaacaagattaaccttgctgcccttgggctaactgaagatgagagagaggctgccacatcatacctgggcggagatgaagacaagtcacagaagtttgagtaattaaaaattcgggacaagtgaaaatgtctttccccgaaggcaaggatatcttgatgatgggaagtgaggcagctaagttggcagaggcttatcagcaatcaatcaagaattccacttctgtgagaagatccattagtggtgaccctgttagcacagtgtctgaaaaagttccattaccaccactatgtagctcagaaacttctagaggagcatgcataagacccacaaaatcaaccctgcctcccatcaaggaagtggagtccatctacccaaaactacccacagctccccctgatgccatgattgagaccgcccaccccgtaggggctccaaagaaggcacagaaaagggtgaaatttgagagttcaaaagcaggcaaatacaccaagctagaagaagaagccctggagctcctgtccgaccctgatgaagataatgatgagaaatcatctgtacttacatttgaggagaaggacaatgcaccatcatctatagaggcgaggttggaggctatagaggaaaaactcagcatgatattggggatgttaaagaccctgagcatagccaccgcagggccaaccgcagcgagagacggaatacgtgatgcaatggtgggagttagagaagagttgatcaatagcattatggcggaagctaaagggaagattgcagaaatgataaaggaagaagatgcacagagggcaaaaataggagatgggagtgtgaaactaacagagaaagctcgggaattaaacaggatgcttgaggatcaaagctcgagtggtgaatctgagacagaaagtgaagagactgaaccagatactgatggtgaaaatgatgatatatacagctttgatatgtagttatgaaaaaatgggacaagtaaacatggagtcctatattatagatacctatcaaggtgtgccctacacagctgctgttcaagtcgatctagttgagaaggacaacaaccctgcaaaactgacggtttggtttcctttatttcagtccagcactcctgctccagtgctgctggaccagctaaaaactctgtcaatcactacacaatatactgcttcccctgaaggaccagtgctacaagtaaatgctactgcacaaggtgcagccatgtcagctctgcccaagaagttttcagttagtgctgctgtagcacttgatgagtacagcaaacttgactttggggtactcacagtttgcgatgtaagggcagtttacctcaccactctgaagccatacggtatggtgtctaagattgtaacaaacatgaacaccgttgggcgcaaaactcacgacctgatagccctttgtgacttcattgacatggagagaggaataccagtaactatccctgcctacataaaggcggtatccatcaaagattctgagtcagctacagttgaagctgcgatcagtggtgaagctgaccaggctatcacccaggcaagaatagctccgtatgcagggttgattctggtcatgaccatgaacaaccccaaagggatattcaggaaactcggtgcaggaacacaagttattgtggagttagggccttatgtgcaggctgagagcttagggaaaatatgtaaaacatggaaccaccagagaaccaggtatgtcctgaagtcccgctaaaactggcaaacaaaagtctactattcttctgtagtttaataaaaatatggggcaagtaaaatgtacctcaaactgctactaataatttatttggtggttggggccagtgggaagatacaagaaacttacagtgaagaatcatgcagcactgtaaccaggggttacaaaagtgtgctcagaacgggttggtatacaaatgtgttcaacctagaaatagggaatgtggagaacataacatgtaatgatggtcctagccttatcagcactgaattatcactaactcagaatgccttgcaagagcttagaactgtttctgccgatcagattacaaaggagaatcgaatcctttcccataggaagaagaggtttgtgttgggtgcaattgcccttggagcggccaccacagctgctgtaacagccggcgtagctttagctaaaacaataagattagaaggggaggtgaaagccatcaagctagctttgcgcagtacaaatgaggctgtgtccacattaggcaatggcgttcgcatcttagcaacagctgttaatgacctaaaagaatttataagcaagaaattaacccctgcaataaaccaaaacaaatgcaacatagcagacataagaatggcaatcagcttcggacagaacaacagaaggtttctaaatgtggtgagacaattttctgacagtgcaggaataacttccgcagtgtctttagatcttatgacagatgcagaattggttaaagccatcaaccgaatgccaacttcgtctggccagattagcctcatgctgaacaatagagcaatggttaggaggaaagggtttggaatacttataggtgtttacggggggactgtagtgtatatggtgcaactcccaatatttggagttatagaaacaccctgctggagagtggtagctgcacccctctgtagacatgagagggagagttatgcttgtctgctgcgggaagatcaggggtggtactgcactaatgcaggatcaactgcttactacccaaatgaagatgactgtgaggtaagagatgactatgtgttctgtgatactgcagcaggtataaatgttgcatcagaggtggagcagtgcaaccaaaacatatcaacctctacttacccttgcaaagttagcacagggagacaccctgtaagcatggtagccttaacccctttgggaggtttagtatcatgttatgaaggtgtgagttgctccattggcagctacaaagtcggaatcattaaacaactcaacaaaggatgcacacacatacccaataatgaggcagacacaataactatagataacacaatttatcaactcagcaaggtggtgggtgagcaaaggaccatcaaaggagctccggtggtaaataacttcaaccctctactgttccctgaggatcaatttaacgtagccttagatcaagtatttgagagtgttgataaatcaaaagacctgattgacaagtctaatgacttactggacatagaagttaagagtaatataggtgctgcattagccatcacaattttagtagtgcttagcatgtcaatcatagtgggcatagcttactatgtggttaaaaagaggaaagccaaagcacccaatggatatcctaaaacaacagggcaaagcaacatgggctacatcagttgagttacataaaaaatgggacaagtaaagatgtccagaaggaatccctgcagatatgaaacaagaggcaagtgcaaccgagggtcctcatgtacatttaaccacaattactggtcttggccagaccatgtattgttagtgcgggctaattatatgctgaatcagctagttaggaacacagataggactgacgggttatctctcatatcaggagcagggagagaagataggacacaggattttgtgctcggttctgccaatgttgtccagaattatatagaagggaatgcaaccataacaaaatctgcagcttgttatagcttgtacaacattatcaagcaacttcaagagaatgatgtgaaatctgcacgagacctgatggtagatgaccccaagcatgttgccctgcataaccttgtcttgtcctatatagacatgagtaagaatccagcaaatctcataaacagtctcaagagactacctaaagaaaaacttaaaaaattggccaaaatcataattcagctttctgctggttcagaaggagaaaatgccaatagtaataccctgcaaaagggtgacagcagtaattaagtgcaacacctcaggtgtgtgcctgtttaagaggacatatgagcataacatcattagccttggtgatctgatagaagatgtagcgaggatgattattatagatcacataaatagaaaacaatgcaatgaatgtagaaaagattttgaatttgtagcggtttacacatcttacacttagttatataaaaacaattgagcagctacccccaaagaaagatggggcaagtcaagatgacctccactgtcaaccttggatcaagcacatcctcaagatggactatagcaaagtcgcggtgcatgctgtgtcttcggactatgatgaattgtgctgttgttatatgtgcagtgctagttttgatcttccttgttgctacgataggcttgtctgtgaagttggctgtcacaataaaagaaaggaatacttgccagctgaggctatcagagctgagcacaactacagcatcaatcctgagatcaacaaatcagccatatccaggagggtcaacaagcacacccaaactgacaactgtcacatctattaccgatctcacccaccagtgtcctcaaaggaaagagttatgcaatggaacaataacatacaaaaactctgatggatgcctggatgaaaaagaaggagaatccattgactgtatagagctcatagccagatgtgtggaaactctatgcgaccccaaccctaactacaaccactgtatgtgcaccaagaacagcactgggctctggtgttgttacaattagagaaggggtaaacccatcatgcactcaaccactgcaaccagcatatgaatatatacatacaagtagattagttaattaaaatcagaagacgggacaagtatccagatggggtcagagctctacaccatagagggggtgagctcatctgaaatagtcctcaagcaagtcctcagaaggagcaaaagaatactgttaggactggtgttatcagccttaggcttgacgctcactagcactattgttatatctatttgtattagtgtagaacaggtcaaattacgacagtgtgtggacacttattgggcggaaaatggatccttacatccaggacagtcaacagaaaatactccaacaagagataagactgcaacaaaagaccctagaagattacaggcgactggagcaggaaagtttgagagctgtgggtatgtgcaagttgttgatggtgatatgcatgatcacagttatgctgtactgggtggtgttgattgtttgggcttattggctctttgtgaatcaggaccaatttgtcagggagatacttggtctgaaggcggaaacttctgccgatgcactttttcttcccatggggtgagttgctgcaaaaaacccaaaagcgaggcaaccactgcccagaggaaccccaaaccagccaacagcaaatcaactcctccggtacattcagacagggccagcaaagaacataattcctcccaaggggaacaaccccgaagggagccaaccagcagcaatacaactattgctggcaccccatcaacagaggacactgctaagccaacgatcagcaaacctaaactcaccatcaggccctcgaaaagaggttcatccagcagcacaaaggcagcctccagaacccccaaccacaagaccaacaccagaggcaccagcaagacgaccgaccagagactccgcaccggacccactcctgaaaggcccagacaaacccacagcacagcaaccccaccccccacaaccccaatccacaagggccgggccccaacccccaaaccaacaacagacctcaaggtcaacccaagggaaggcggcataagcccaactgcaatacagaaaaacccaaccacacaaagtaatctcgtcgactgcacactgtctgatccaaatgagccaccaaggatttgttaccaggtaggaacttacaatcctagtcaatcaggaacctgcaacatagaggttccaaaatgttccacttatgggtatgcttgtatggctacattatatgacaccccattcaactgctggcgcagaaccaggagatgcatctgtgattccggaggggagctgattgagtggtgctgtactagtcaataaaaagtaacctttcacatctaaaataaagcaaaaagaagaaaaaagaagaaagaaggaaagaaagaagaagaatagcacacaacagaaaggagggaccaatatggacccatccaatgagcacacggtgaatgtgtatctgcctgactcctatttaaaaggagtcatctctttcagcgaaacaaatgcaattggatcttgcattataggtcgaccatacctaaaggatgactacactgctcatgttgcaaccacaagtcctgtcattgagcaccaaagattaaaaagcttgtttagtggtctaacaatcagcaagaactatcaatttgttgaacccctcatgatgcagaatgagctgctcaagatctctgccggtacaaaactcaagaagctaaagaaatggctaggtcgaagcaaagatattagtgagatcaagctgaagatggttgcagactggctcaaacttgcacatactcctgggaaagggaaagtgattgactgtatccaggtagacaacctacctgaatggctagagcaatggtttgattcatggttgatgcttaatgaactgatacaagcttataggtgcctggaagtgtcacagaccagtgctatcctccgtaaaagctgttggaatttcttctttgctatatcatcatttggctgcatcttgataagccggaaaagtaaaaggatttgtttctgcacctataaccagttcttgacatggaaagatttggcacttagcaggttcaatgctaacctgtgtgtctgggttagcaactgtttaaatagtactcaagaaggactaggactccggagcaaacttgttggagaactcctaaatcgattatatcatgagactgatgagttgttgagtggtactggcaatgagggattctggatagttaaggaattcgagggattcatcatgagtgaaattctgaggataactgaacatgcacagtttagtgtacgctttagaaacacactattaaatggaatcgtagaaaaaattgggaaaatgcgtgttgcccacagtcagcgaacttccaacactgccatagaaaatcatagatatcccggagagcaaagtttgttggagaaaacaggaagagtgctcaagattataaagctactagtccaaaacgatatgtccaatgcagcagagatgtattttatctttagaatattcggtcaccctatggtagaagaaagggaggccatggatgctgttcgggagaacagtgaggccactaaaatattgagcctcagggctctgacagagatgagaggagcttttatcctaagagtgatcaaaggttttgtaacaaattacaagaggtggccaagaataaagaacccttctacattgagtagcaggtggcgaatgtatatgagggctaaaacatatcctagtcaacttgagctatgtgctgaagacttcttagagttagcaggtatcagcttctgccaagaattctatgttcccaacaggaccagcctagaaatggtactcaatgacaaggcaatctcccctccaaagtctttgatctggtctgtatatccaaaaaattatttacctccttctgtacaggagcaatttcggttagaatctctggggaaagcagaacatgaaaaaactagacgtgtactagagttttacttgaaggatgccaactttaaccaagagaacttgaaaaagtatgtagtgttacagcactaccttaatgataaagagcatgttgtctctttgactggaaaagaaagggaattaagtgtggggaggatgtttgccatgcagccaggaaaacaaagacaagtgcagatacttgcagaaaaactgttatctgataatatagtaccatttttcccagaaactcttacaagatatggagacctggagttacaaagaattatggagcttaaatctgaactatcttcagtgaaagctaggaagagtgatagctacaacaattatatagcccgggcatcaatagtaacagatcttagcaagttcaaccaagcatttcggtatgaaacaacatcagtttgtgctgatgttgccgacgagctccatggtactcagagcctattttgctggttacatctaacagtttccgccaccactatgatatgcacttataggcatgcaccacctgacaccaaagggatttatgatattgactcaatccctgagcagagtgggttgtatagataccacatgggaggaattgaggggtggtgtcaaaaaatgtggaccatggaggcaatatctctccttgatgttgtgtcggttaagaacagggttcagctaacatcactcctaaatggtgacaatcagtccatagatgttagtaaacctgtgaggcttattggtactcagactgagatacaagcagactacagtttagcaattaaaatgttgactgcagtcagggatgcttattctgacataggacacaaattaaaagaaggtgaaacttatgtgtcaagggatctgcaattcatgagtaagactatacagtctgaaggggtcatgtacccagcctcaattaagaaagttctgagagttggcccatggataaacactatactagacgatatcaagacaagcatggaagccataggtagcctttgtcaggagttggagttccggggggaatcatttactacaagcttgataatacggaatttttggttatatgtccaatgttttgtagaagcaaagagacacagtttagcaggtagtcagatatctaatgaattaaatagaacacttaccaaagtaatgaaatttttccaagtaggcagtgagtcagacacagtaaatctttatatgaatataccgatgcagctgggaggcggagaccctgttgttgtgtataggagcttctataggcggacacctgacttcctaacggaagtactaactcatgtagagttgttactgacatcacttaatgttgatagaggtgtttacatgaggttcttcctcacactaatgaacacctgcaaaaacgacaatgcaacattaactacactcatgagggatcctcaggctattgggtcagaaaggcaggctaaaataacaagtgagatcaatcgtacagcagtcacaagtgtactaagcctggctcctaaccagctttttagtgatagtgcagtacattttagccagaatgaggaagaaattggtactgttatgcaagatgtagggcctgtataccctcatggcttaagggtcatatatgaagcattcccattccacaaagctgaaaaagtagttaacatgatagcaggtaccaaatctattacaaatatattgcaaaggacatccgccataagtggtttagacattgatagagcagttcatatgatgttgctcaatctaggactgttagggaggatattagagtcaggacctgtcacagacaccattgagttgcgtcacaacaataggatactttgttgtcaattatctaagcgcatacgggaaacatcatgggacggaatagaaattgtaggagtgtcatcccctagtatgttatcatgcttggatataaactatgtaacagcagcacagaggccaggagtattaatagagaagttctcggcagaaaagactacaagagggaagagagggcctaaggctccatgggttggatctagcacgcaggagaaaaaattaacagcagtgtacaataggcaagccttatcaaaagagcagagagatcagttagaaacaataggtaagattagatgggtgtacagaggggtaacaggactacggagacttttagatctagtctgcatgggaaccttaggacttccctacaagctgataaaacccctgttaccaagattcatgagtgtcaatttcctgcatcgtcttgcagtgagcagtagaccgatggagtttccttcatcagtcccagcataccgcacaactaatttccactttgacacaagccccatcaataaaagacttagtgagagatttggaaatgaggacattaacttggtcttccaaaatgctataagctgtgggattagtgtcatgtgtatagtggagcaattaacagggaagagcccaaagttagtcgtgctagagccaattgtggaagatatagacataatgtctgctcctaatttccaagggagggtaaatttcaagagtgtgaagaaaattgtggctgatcagcacatcttcaaccctgatcatatcagcctcacaatgctggggagactcctactgcccactataagaagcaactcaaatgcagagagaattgcaactgagaatttctttaatgggaacaatatagtagaggtgctatccagttgccttgcatgccattggtgtacaattttgttgctcctcaccatggagaatagtattttccagaaagagtggggtgatgggtttgttacagaccatgcctttataaactttacatggttcctgatgagcttcaaaacatatttgctctgccactgggggggtgacgatgaaggagagctaaacattgtagaagacccaattgacaggctagctagaatagacaatagtttttggcgtatgatgagcaaggttttccttgaaccaaaagtcaagagacgattgatgctgtatgacaccacaatcctaaatgtctttggcagtatcagttttaaaaactggttcattgggaagcttaggtcagcagattatacagaaataccttggatagtaaatgcagaaggagacattgtggaacagaggcccgtcacggagtaccttaaaaccatggcagctgggactaatgtcaaagtgataatgctgagttactcagacatggcacatgctatgacacggctgcttagatgcaaaaacatgcaggacaatgttccgaccattaagaaggcagctaccccaaccgatgttacaccagctgtggacccaacaagagctttgttgttgtaccctaaagttacctttagcaaactgacaactttcaatgcggcatgtactagcctacgacaacccgggggaaacttgtccaaaaattacataactttgctgccttggcaccatgtgaaccgatacaatttcgtgcatagttctacagggtgcaaggtcagcataagaagctgtctcggtaaattggtggcaaaactagacctcagagttatttactttattggtgaaggtgcagggaacttgatgtcaaggacagcatgtgaatatcctggcttaaaatttgtctacaggagcctaaaagatgctaacgatcatcaccaccccacagaatatgtgcgtgtcatggggagcatcagcagaatagtggacttaggtgaggggttagctatggaagcgactgatgccacgaggagagagcattgggatttcatacacagagtgagcaaagaacccttgttactgacagtgtgtgatgctgaactcaaagaccctaataacgtacttttattaatcatacagtggagaaagcatgtgctttcgtgtaaaatatgcacatcatacgggacaagtgtgtacatgctgatcaaatatcatgcccagcaagagccaaaaaaactcccacattttgtacgggtggtttctatatacgttatgcagggtagtaaagtctccggttcagagtgttatctgctcattactctcggacatcagaacacagccccttgctatggagaggtgcgtgctgcaaagtcagtaatggctctcactgaagtctacaagaatcctataaggctagacaaaatagctgtcgaaatcaatctaaagtctttagccccaggattatctttaccaatcacagaggtgagcatgttgaagtacctttcaaacctaacacacttggacaactccaaaaatattagtgccaccattgggggcagcagagttgtagaaaagaaatgggcaatgtgcaaagttgcaaatgttgtaagatggctatgtcatgtcagtaggatgcccaaaggcgacctgaattatgatttctttgaagtaattgagagcacctatccggatatggtgaaattgctagacaaccttaactcttcagagttgaagaaactagtgaaagtgacagggtacatactgagcacaaaatagaccataaagaatgatcacacagtcgtacacagcaggtaagttgtaactaaaaatccaatagggaaaaggggtgcattagttataaaaaacaaaaaacaaaaattaaaaatgtattg

SEQ ID NO: 11 - RNA, 13472 nt, Avian metapneumovirus; identical to SEQ ID NO: 10
  source 1..13472: mol_type = genomic RNA

SEQ ID NO: 12 - RNA, 13472 nt, Avian metapneumovirus; identical to SEQ ID NO: 10
  source 1..13472: mol_type = genomic RNA

SEQ ID NO: 13 - RNA, 13474 nt, Avian metapneumovirus
  source 1..13474: mol_type = genomic RNA
tcaagtcacaatagaaaagaaaaatgggacaagtaaaaatgtctcttgaaagtattagactcagtgatctagagtacaagcatgcaatccttgatgagtcacaatatacaataaggagagatgttggtgctacaactgctatcacaccctctgaactacagccaaaggtatccaccctgtgtggaatgattttatttgctaagcatgcagattatgaaccagcagcagaagttggcatgcagtacatcagcactgcattaggagctgataagacacagcagatactcaaaagctctggcagtgaagtgcaaggggtcatgaccaagatagtgacactccctgcagaaggtcccatacgaaagcgggaagtacttaatatccatgacattgggcccgcatgggcagacaatgtagaacggactgcaagggagaccatgagcctaatggtaaaagagaaagctcaaatacccaaaaaccaaaagccgtcagctcttgatgcacctgtgatcttactatgtattggggcattaatattcactaagcttgcatccacagtagaggttgggctggagactgctattaggagagcctctcgggtgttaagtgatgcaatatcacgttaccctaggatggacataccacggattgccaaatccttctttgagctatttgaaaagaaagtgtactataggaaccttttcatagagtacggtaaggcacttgggagtacgtcttccgggagtaggatggagagcctctttgttaacatcttcatgcaagcttatggagctgggcagactatgctaagatggggtgttgtggcaagatcatccaataacatcatgttgggccatgtgtctgtgcaggcagagttaaggcaggtctcagaagtgtacgatcttgttaggaaaatgggtcctgaatcaggcctcctccacttgaggcaaagtccaaaagcaggcttactatcattaacaagttgccccaactttgcaagtgttgttttggggaatgcagctggcctaggcatcattgggatgtataagggcagagcacccaacctggagttgttttctgcagcagaaagttatgctagatccctcaaagagagcaacaagattaaccttgctgcccttgggctaactgaagatgagagagaggctgccacatcatacctaggcggagatgaagacaagtcacagaagtttgagtaattaaaaaaattcgggacaagtgaaaatgtctttccccgaaggcaaggatatcttgatgatgggaagtgaggcagctaagttggcagaggcttatcagcaatcaatcaagaattccacttctgtgagaagatccattagtggtgaccctgttagcacagtgtctgaaaaagttccattaccaccactatgtagctcagaaacttctagaggagcatgcataagacccacaaaatcaaccctgcctcccatcaaggaagtggagtccatctacccaaaactacccacagctccccctgatgccatgattgagaccgcccaccccgtaggggctccaaagaaggcacagaaaagggtgaaatttgagagttcaaaagcaggcaaatacaccaagctagaagaagaagccctggagctcctgtccgaccctgatgaagataatgatgagaaatcatctgtacttacatttgaggagaaggacaatgcaccatcatctatagaggcgaggttggaggctatagaggaaaaactcagcatgatattggggatgttaaagaccctgagcatagccaccgcagggccaaccgcagcgagagacggaatacgtgatgcaatggtgggagttagagaagagttgatcaatagcattatggcggaagctaaagggaagattgcagaaatgataaaggaagaagatgcacagagggcaaaaataggagatgggagtgtgaaactaacagagaaagctcgggaattaaacaggatgcttgaggatcaaagctcgagtggtgaatctgagacagaaagtgaagagactgaaccagatactgatggtgaaaatgatgatatatacagctttgatatgtagttatgaaaaaatgggacaagtaaacatggagtcctatattatagatacctatcaaggtgtgccctacacagctgctgttcaagtcgatctagttgagaaggacaacaaccctgcaaaactgacggtttggtttcctttatttcagtccagcactcctgctccagtgctgctggaccagctaaaaactctgtcaatcactacacaatatactgcttcccctgaaggaccagtgctacaagtaaatgctactgcacaaggtgcagccatgtcagctctgcccaagaagttttcagttagtgctgctgtagcacttgatgagtacagcaaacttgactttggggtactcacagtttgcgatgtaagggcagtttacctcaccactctgaagccatacggtatggtgtctaagattgtaacaaacatgaacaccgttgggcgcaaaactcacgacctgatagccctttgtgacttcattgacatggagagaggaataccagtaactatccctgcctacataaaggcggtatccatcaaagattctgagtcagctacagttgaagctgcgatcagtggtgaagctgaccaggctatcacccaggcaagaatagctccgtatgcagggttgattctggtcatgaccatgaacaaccccaaagggatattcaggaaactcggtgcaggaacacaagttattgtggagttagggccttatgtgcaggctgagagcttagggaaaatatgtaaaacatggaaccaccagagaaccaggtatgtcctgaagtcccgctaaaactggcaaacaaaagtctactattcttctgtagtttaataaaaatatggggcaagtaaaatgtacctcaaactgctactaataatttatttggtggttggggccagtgggaagatacaagaaacttacagtgaagaatcatgcagcactgtaaccaggggttacaaaagtgtgctcagaacgggttggtatacaaatgtgttcaacctagaaatagggaatgtggagaacataacatgtaatgatggtcctagccttatcagcactgaattatcactaactcagaatgccttgcaagagcttagaactgtttctgccgatcagattacaaaggagaatcgaatcctttcccataggaagaagaggtttgtgttgggtgcaattgcccttggagtggccaccacagctgctgtaacagccggcgtagctttagctaaaacaataagattagaaggggaggtgaaagccatcaagctagctttgcgcagtacaaatgaggctgtgtccacattaggcaatggcgttcgcatcttagcaacagctgttaatgacctaaaagaatttataagcaagaaattaacccctgcaataaaccaaaacaaatgcaacatagcagacataagaatggcaatcagctttggacagaacaacagaaggtttctaaatgtggtgagacaattttctgacagtgcaggaataacttccgcagtgtctttagatcttatgacagatgcagaattggttaaagccatcaaccgaatgccaacttcgtctggccagattagcctcatgctgaacaatagagcaatggttaggaggaaagggtttggaatacttataggtgtttacggggggactgtagtgtatatggtgcaactcccaatatttggagttatagaaacaccctgctggagagtggtagctgcacccctctgtagacatgagagggagagttatgcttgtctgctgcgggaagatcaggggtggtactgcactaatgcaggatcaactgcttactacccaaatgaagatgactgcgaggtaagagatgactatgtgttctgtgatactgcagcaggtataaatgttgcatcagaggtggagcagtgcaaccaaaacatatcaacctctacttacccttgcaaagttagcacagggagacaccctgtaagcatggtagccttaacccctttgggaggtttagtatcatgttatgaaggtgtgagttgctccattggcagcaacaaagtcggaatcattaaacaactcaacaaaggatgcacacacatacccaataatgaggcagacacaataactatagataacacaatttatcaactcagcaaggtggtgggtgagcaaaggactatcaaaggagctccggtggtaaataacttcaaccctctactgttccctgaggatcaatttaacgtagccttagatcaagtatttgagagtgttgataaatcaaaagacctgattgacaagtctaatgacttactggacatagaagttaagagtaatataggtgctgcattagccatcacaattttagtagtgcttagcatgtcaatcatagtgggcatagcttactatgtggttaaaaagaggaaagccaaagcacccaatggatatcctaaaacaacagggcaaagcaacatgggctacatcagttgagttacataaaaaatgggacaagtaaagatgtccagaaggaatccctgcagatatgaaacaagaggcaagtgcaaccgagggtcctcatgtacatttaaccacaattactggtcttggccagaccatgtattgttagtgcgggctaattatatgctgaatcagctagttaggaacacagataggactgacgggttatctctcatatcaggagcagggagagaagataggacacaggattttgtgctcggttctgccaatgttgtccagaattatatagaagggaatgcaaccataacaaaatctgcagcttgttatagcttgtacaacattatcaagcaacttcaagagaatgatgtgaaatctgcacgagacctgatggtagatgaccccaagcatgttgccctgcataaccttgtcttgtcctatatagacatgagtaagaatccagcaaatctcataaacagtctcaagagactacctaaagaaaaacttaaaaaattggccaaaatcataattcagctttctgctggttcagaaggagaaaatgccaatagtaataccctgcaaaagggtgacagcagtaattaagtgcaacacctcaggtgtgtgcctgtttaagaggacatatgagcataacatcattagccttggtgatctgatagaagaagtagcgaggatgattattatagatcacataaatagaaaacaatgcaatgaatgtagaaaagattttgaatttgtagcggtttacacatcttacacttagttacataaaaacaattgagcagctacccccaaagaaagatggggcaagtcaagatgacctccactgtcaaccttggatcaagcacatcctcaagatggactatagcaaagtcgcggtgcatgctgtgtcttcggactatgatgaattgtgctgttgttatatgtgcagtgctagttttgatcttccttgttgctacgataggcttgtctgtgaagttggctgtcacaataaaagaaaggaatacttgccagctgaggctatcagagctgagcacaactacagcatcaatcctgagatcaacaaatcagccatatccaggagggtcaacaagcacacccaaactgacaactgtcacatctattaccgatctcacccaccagtgtcctcaaaggaaagagttatgcaatggaacaataacatacaaaaactctgatggatgcctggatgaaaaagaaggagaatccattgactgtatagagctcatagccagatgtgtggaaactctatgcgaccccaaccctaactacaaccactgtatgtgcaccaagaacagcactgggctctggtgttgttacaattagagaaggggtaaacccatcatgcactcaaccactgcaaccagcatatgaatatatacatacaagtagattagttaattaaaatcagaagacgggacaagtatccagatggggtcagagctctacaccatagagggggtgagctcatctgaaatagtcctcaagcaagtcctcagaaggagcaaaagaatactgttaggactggtgttatcagccttaggcttgacgctcactagcactattgttatatctatttgtattagtgtagaacaggtcaaattacgacagtgtgtggacacttattgggcggaaaatggatccttacatccaggacagtcaacagaaaatactccaacaagagataagactacaacaaaagaccctagaagattacaggcgactggagcaggaaagtttgagagctgtgggtatgtgcaagttgttgatggtgatatgcatgatcacagttatgctgtactgggtggtgttgattgtttgggcttattggctctttgtgaatcaggaccaatttgtcagggagatacttggtctgaaggcggaaacttctgccgatgcactttttcttcccatggggtgagttgctgcaaaaaacccaaaagcgaggcaaccactgcccagaggaaccccaaaccagccaacagcaaatcaactcctccggcacattcagacagggccagcaaagaacataattcctcccaaggggaacaaccccgaagggagccaaccagcagcaatacaactattgctggcaccccatcaacagaggacactgctaagccaacgatcagcaaacctaaactcaccatcaggccctcgaaaagaggttcatccagcagcacaaaggcagcctccagaacccccaaccacaagaccaacaccagaggcaccagcaagacgaccgaccagagaccccgcaccggacccacccctgaaaggcccagacaaacccacagcacagcaaccccaccccccacaaccccaatccacaagggccgggccccaacccccaaaccaacaacagacctcaaggtcaacccaagggaaggcggcacaagcccaactgcaatacagaaaaacccaaccacacaaagtaatctcgtcgactgcacactgtctgatccaaatgagccaccaaggatttgttaccaggtaggaacttacaatcctagtcaatcaggaacctgcaacatagaggttccaaaatgttccacttatgggtatgcttgtatggctacattatatgacaccccattcaactgctggcgcagaaccaggagatgcatctgtgattccggaggggagctgattgagtggtgctgtactagtcaataaaaagtaacctttcacatctaaaataaagcaaaaagaagaaaaaagaagaaagaaggaaagaaagaagaagaatagcacacaacagaaaggagggaccaatatggacccatccaatgagcacacggtgaatgtgtatctgcctgactcctatttaaaaggagtcatctctttcagcgaaacaaatgcaattggatcttgcattataggtcgaccatacctaaaggatgactacactgctcatgttgcaaccacaagtcctgtcattgagcaccaaagattaaaaagcttgtttagtggtctaacaatcagcaagaactatcaatttgttgaacccctcatgatgcagaatgagctgctcaagatctctgccggtacaaaactcaagaagctaaagaaatggctaggtcgaagcaaagatattagtgagatcaagctgaagatggttgcagactggctcaaacttgcacatactcctgggaaagggaaagtgattgactgtatccaggtagacaacctacctgaatggctagagcaatggtttgattcatggttgatgcttaatgaactgatacaagcttataggtgcctggaagtgtcacagaccagtgctatcctccgtaaaagctgttggaatttcttctttgctatatcatcatttggctgcatcttgataagccggaaaagtaaaaggatttgtttctgcacctataaccagttcttgacatggaaagatttggcacttagcaggttcaatgctaacctgtgtgtctgggttagcaactgtttaaatagtactcaagaaggactaggactccggagcaaacttgttggagaactcctaaatcgattatatcatgagactgatgagttgttgagtggtactggcaatgagggattctggatagttaaggaattcgagggattcatcatgagtgaaattctgaggataactgaacatgcacagtttagtgtacgctttagaaacacactattaaatggaatcgtagaaaaaattgggaaaatgcgtgttgcccacagtcagcgaacttccaacactgccatagaaaatcatagatatcccggagagcaaagtttgttggagaaaacaggaagagtgctcaagattataaagctactagtccaaaacgatatgtccaatgcagcagagatgtattttatctttagaatattcggtcaccctatggtagaagaaagggaggccatggatgctgttcgggagaacagtgaggccactaaaatattgagcctcagggctctgacggagatgagaggagcttttatcctaagagtgatcaaaggttttgtaacaaattacaagaggtggccaagaataaagaacccttctacattgagtagcaggtggcgaatgtatatgagggctaaaacatatcctagtcaacttgagctatgtgctgaagacttcttagagttagcaggtatcagcttctgccaagaattctatgttcccaacaggaccagcctagaaatggtactcaatgacaaggcaatctcccctccaaagtctttgatctggtctgtatatccaaaaaattatttacctccttctgtacaggagcaatttcggttagaatctctggggaaagcagaacatgaaaaaactagacgtgtactagagttttacttgaaggatgccaactttaaccaagagaacttgaaaaagtatgtagtgttacagcactaccttaatgataaagagcatgttgtctctttgactggaaaagaaagggaattaagtgtggggaggatgtttgccatgcagccaggaaaacaaagacaagtgcagatacttgcagaaaaactgttatctgataatatagtaccatttttcccagaaactcttacaagatatggagacctggagttacaaagaattatggagcttaaatctgaactatcttcagtgaaagctaggaagagtgatagctacaacaattatatagcccgggcatcaatagtaacagatcttagcaagttcaaccaagcatttcggtatgaaacaacatcagtttgtgctgatgttgccgacgagctccatggtactcagagcctattttgctggttacatctaacagtttccgccaccactatgatatgcacttataggcatgcaccacctgacaccaaagggatttatgatattgactcaatccctgagcagagtgggttgtatagataccacatgggaggaattgaggggtggtgtcaaaaaatgtggaccatggaggcaatatctctccttgatgttgtgtcggttaagaacagggttcagctaacatcactcctaaatggtgacaatcagtccatagatgttagtaaacctgtgaggcttattggtactcagactgagatacaagcagactacagtttagcaattaaaatgttgactgcagtcagggatgcttattctgacataggacacaaattaaaagaaggtgaaacttatgtgtcaagggatctgcaattcatgagtaagactatacagtctgaaggggtcatgtacccagcctcaattaagaaagttctgagagttggcccatggataaacactatactagacgatatcaagacaagcatggaagccataggtagcctttgtcaggagttggagttccggggggaatcatttactacaagcttgataatacggaatttttggttatatgtccaatgttttgtagaagcaaagagacacagtttagcaggtagtcagatatctaatgaattaaatagaacacttaccaaagtaatgaaatttttccaagtaggcagtgagtcagacacagtaaatctttatatgaatataccgatgcagctgggaggcggagaccctgttgttgtgtataggagcttctataggcggacacctgacttcctaacggaagtactaactcatgtagagttgttactgacatcacttaatgttgatagaggtgtttacatgaggttcttcctcacactaatgaacacctgcaaaaacgacaatgcaacattaactacactcatgagggatcctcaggctattgggtcagaaaggcaggctaaaataacaagtgagatcaatcgtacagcagtcacaagtgtactaagcctggctcctaaccagctttttagtgatagtgcagtacattttagccagaatgaggaagaaattggtactgttatgcaagatgtagggcctgtataccctcatggcttaagggtcatatatgaagcattcccattccacaaagctgaaaaagtagttaacatgatagcaggtaccaaatctattacaaatatattgcaaaggacatccgccataagtggtttagacattgatagagcagttcatatgatgttgctcaatctaggactgttagggaggatattagagtcaggacctgtcacagacaccattgagttgcgtcacaacaataggatactttgttgtcaattatctaagcgcatacgggaaacatcatgggacggaatagaaattgtaggagtgtcatcccctagtatgttatcatgcttggatataaactatgtaacagcagcacagaggccaggagtattaatagagaagttctcggcagaaaagactacaagagggaagagagggcctaaggctccatgggttggatctagcacgcaggagaaaaaattaacagcagtgtacaataggcaagccttatcaaaagagcagagagatcagttagaaacaataggtaagattagatgggtgtacagaggggtaacaggactacggagacttttagatctagtctgcatgggaaccttaggacttccctacaagctgataaaacccctgttaccaagattcatgagtgtcaatttcctgcatcgtcttgcagtgagcagtagaccgatggagtttccttcatcagtcccagcataccgcacaactaatttccactttgacacaagccccatcaataaaagacttagtgagagatttggaaatgaggacattaacttggtcttccaaaatgctataagctgtgggattagtgtcatgtgtatagtggagcaattaacagggaagagcccaaagttagtcatgctagagccaattgtggaagatatagacataatgtctgctcctaatttccaagggagggtaaatttcaagagtgtgaagaaaattgtggctgatcagcacatcttcaaccctgatcatatcagcctcacaatgctggggagactcctactgcccactataagaagcaactcaaatgcagagagaattgcaactgagaatttctttaatgggaacaatatagtagaggtgctatccagttgccttgcatgccattggtgtacaattttgttgctcctcaccatggagaatagtattttccagaaagagtggggtgatgggtttgttacagaccatgcctttataaactttacatggttcctgatgagcttcaaaacatatttgctctgccactgggggggtgacgatgaaggagagctaaacattgtagaagacccaattgacaggctagctagaatagacaatagtttttggcgtatgatgagcaaggttttccttgaaccaaaagtcaagagacgattgatgctgtatgacaccacaatcctaaatgtctttggcagtatcagttttaaaaactggttcattgggaagcttaggtcagcagattatacagaaataccttggatagtaaatgcagaaggagacattgtggaacagaggcccgtcacggagtaccttaaaaccatggcagctgggactaatgtcaaagtgataatgctgagttactcagacatggcacatgctatgacacggctgcttagatgcaaaaacatgcaggacaatgttccgaccattaagaaggcagctaccccaaccgatgttacaccagctgtggacccaacaagagctttgttgttgtaccctaaagttacctttagcaaactgacaactttcaatgcggcatgtactagcctacgacaacccgggggaaacttgtccaaaaattacataactttgctgccttggcaccatgtgaaccgatacaatttcgtgcatagttctacagggtgcaaggtcagcataagaagctgtctcggtaaattggtggcaaaactagacctcagagttatttactttattggtgaaggtgcagggaacttgatgtcaaggacagcatgtgaatatcctggcttaaaatttgtctacaggagcctaaaagatgctaacgatcatcaccaccccacagaatatgtgcgtgtcatggggagcatcagcagaatagtggacttaggtgaggggttagctatggaagcgactgatgccacgaggagagagcattgggatttcatacacagagtgagcaaagaacccttgttactgacagtgtgtgatgctgaactcaaagaccctaataacgtacttttattaatcatacagtggagaaagcatgtgctttcgtgtaaaatatgcacatcatacgggacaagtgtgtacatgctgatcaaatatcatgcccagcaagagccaaaaaaactcccacattttgtacgggtggtttctatatacgttatgcagggtagtaaagtctccggttcagagtgttatctgctcattactctcggacatcagaacacagccccttgctatggagaggtgcgtgctgcaaagtcagtaatggctctcactgaagtctacaagaatcctataaggctagacaaaatagctgtcgaaatcaatctaaagtctttagccccaggattatctttaccaatcacagaggtgagcatgttgaagtacctttcaaacctaacacacttggacaactccaaaaatattagtgccaccattgggggcagcagagttgtagaaaagaaatgggcaatgtgcaaagttgcaaatgttgtaagatggctatgtcatgtcagtaggatgcccaaaggcgacctgaattatgatttctttgaagtaattgagagcacctatccggatatggtgaaattgctagacaaccttaactcttcagagttgaagaaactagtgaaagtgacagggtacatactgagcacaaaatagaccataaagaatgatcacacagtcgtacacagcaggtaagttgtaactaaaaatccaatagggaaaaggggtgcattagttataaaaaacaaaaaacaaaaattaaaaatgtattg

SEQ ID NO: 14 - RNA, 13474 nt, Avian metapneumovirus; identical to SEQ ID NO: 13
  source 1..13474: mol_type = genomic RNA

SEQ ID NO: 15 - RNA, 13474 nt, Avian metapneumovirus
  source 1..13474: mol_type = genomic RNA
tcaagtcacaatagaaaagaaaaatgggacaagtaaaaatgtctcttgaaagtattagactcagtgatctagagtacaagcatgcaatccttgatgagtcacaatatacaataaggagagatgttggtgctacaactgctatcacaccctctgaactacagccaaaggtatccaccctgtgtggaatgattttatttgctaagcatgcagattatgaaccagcagcagaagttggcatgcagtacatcagcactgcattaggagctgataagacacagcagatactcaaaagctctggcagtgaagtgcaaggggtcatgaccaagatagtgacactccctgcagaaggtcccatacgaaagcgggaagtacttaatatccatgacattgggcccgcatgggcagacaatgtagaacggactgcaagggagaccatgagcctaatggtaaaagagaaagctcaaatacccaaaaaccaaaagccgtcagctcttgatgcacctgtgatcttactatgtattggggcattaatattcactaagcttgcatccacagtagaggttgggctggagactgctattaggagagcctctcgggtgttaagtgatgcaatatcacgttaccctaggatggacataccacggattgccaaatccttctttgagctatttgaaaagaaagtgtactataggaaccttttcatagagtacggtaaggcacttgggagtacgtcttccgggagtaggatggagagcctctttgttaacatcttcatgcaagcttatggagctgggcagactatgctaagatggggtgttgtggcaagatcatccaataacatcatgttgggccatgtgtctgtgcaggcagagttaaggcaggtctcagaagtgtacgatcttgttaggaaaatgggtcctgaatcaggcctcctccacttgaggcaaagtccaaaagcaggcttactatcattaacaagttgccccaactttgcaagtgttgttttggggaatgcagctggcctaggcatcattgggatgtataagggcagagcacccaacctggagttgttttctgcagcagaaagttatgctagatccctcaaagagagcaacaagattaaccttgctgcccttgggctaactgaagatgagagagaggctgccacatcatacctaggcggagatgaagacaagtcacagaagtttgagtaattaaaaaaattcgggacaagtgaaaatgtctttccccgaaggcaaggatatcttgatgatgggaagtgaggcagctaagttggcagaggcttatcagcaatcaatcaagaattccacttctgtgagaagatccattagtggtgaccctgttagcacagtgtctgaaaaagttccattaccaccactatgtagctcagaaacttctagaggagcatgcataagacccacaaaatcaaccctgcctcccatcaaggaagtggagtccatctacccaaaactacccacagctccccctgatgccatgattgagaccgcccaccccgtaggggctccaaagaaggcacagaaaagggtgaaatttgagagttcaaaagcaggcaaatacaccaagctagaagaagaagccctggagctcctgtccgaccctgatgaagataatgatgagaaatcatctgtacttacatttgaggagaaggacaatgcaccatcatctatagaggcgaggttggaggctatagaggaaaaactcagcatgatattggggatgttaaagaccctgagcatagccaccgcagggccaaccgcagcgagagacggaatacgtgatgcaatggtgggagttagagaagagttgatcaatagcattatggcggaagctaaagggaagattgcagaaatgataaaggaagaagatgcacagagggcaaaaataggagatgggagtgtgaaactaacagagaaagctcgggaattaaacaggatgcttgaggatcaaagctcgagtggtgaatctgagacagaaagtgaagagactgaaccagatactgatggtgaaaatgatgatatatacagctttgatatgtagttatgaaaaaatgggacaagtaaacatggagtcctatattatagatacctatcaaggtgtgccctacacagctgctgttcaagtcgatctagttgagaaggacaacaaccctgcaaaactgacggtttggtttcctttatttcagtccagcactcctgctccagtgctgctggaccagctaaaaactctgtcaatcactacacaatatactgcttcccctgaaggaccagtgctacaagtaaatgctactgcacaaggtgcagccatgtcagctctgcccaagaagttttcagttagtgctgctgtagcacttgatgagtacagcaaacttgactttggggtactcacagtttgcgatgtaagggcagtttacctcaccactctgaagccatacggtatggtgtctaagattgtaacaaacatgaacaccgttgggcgcaaaactcacgacctgatagccctttgtgacttcattgacatggagagaggaataccagtaactatccctgcctacataaaggcggtatccatcaaagattctgagtcagctacagttgaagctgcgatcagtggtgaagctgaccaggctatcacccaggcaagaatagctccgtatgcagggttgattctggtcatgaccatgaacaaccccaaagggatattcaggaaactcggtgcaggaacacaagttattgtggagttagggccttatgtgcaggctgagagcttagggaaaatatgtaaaacatggaaccaccagagaaccaggtatgtcctgaagtcccgctaaaactggcaaacaaaagtctactattcttctgtagtttaataaaaatatggggcaagtaaaatgtacctcaaactgctactaataatttatttggtggttggggccagtgggaagatacaagaaacttacagtgaagaatcatgcagcactgtaaccaggggttacaaaagtgtgctcagaacgggttggtatacaaatgtgttcaacctagaaatagggaatgtggagaacataacatgtaatgatggtcctagccttatcagcactgaattatcactaactcagaatgccttgcaagagcttagaactgtttctgccgatcagattacaaaggagaatcgaatcctttcccataggaagaagaggtttgtgttgggtgcaattgcccttggagtggccaccacagctgctgtaacagccggcgtagctttagctaaaacaataagattagaaggggaggtgaaagccatcaagctagctttgcgcagtacaaatgaggctgtgtccacattaggcaatggcgttcgcatcttagcaacagctgttaatgacctaaaagaatttataagcaagaaattaacccctgcaataaaccaaaacaaatgcaacatagcagacataagaatggcaatcagctttggacagaacaacagaaggtttctaaatgtggtgagacaattttctgacagtgcaggaataacttccgcagtgtctttagatcttatgacagatgcagaattggttaaagccatcaaccgaatgccaacttcgtctggccagattagcctcatgctgaacaatagagcaatggttaggaggaaagggtttggaatacttataggtgtttacggggggactgtagtgtatatggtgcaactcccaatatttggagttatagaaacaccctgctggagagtggtagctgcacccctctgtagacatgagagggagagttatgcttgtctgctgcgggaagatcaggggtggtactgcactaatgcaggatcaactgcttactacccaaatgaagatgactgcgaggtaagagatgactatgtgttctgtgatactgcagcaggtataaatgttgcatcagaggtggagcagtgcaaccaaaacatatcaacctctacttacccttgcaaagttagcacagggagacaccctgtaagcatggtagccttaacccctttgggaggtttagtatcatgttatgaaggtgtgagttgctccattggcagcaacaaagtcggaatcattaaacaactcaacaaaggatgcacacacatacccaataatgaggcagacacaataactatagataacacaatttatcaactcagcaaggtggtgggtgagcaaaggactatcaaaggagctccggtggtaaataacttcaaccctctactgttccctgaggatcaatttaacgtagccttagatcaagtatttgagagtgttgataaatcaaaagacctgattgacaagtctaatgacttactggacatagaagttaagagtaatataggtgctgcattagccatcacaattttagtagtgcttagcatgtcaatcatagtgggcatagcttactatgtggttaaaaagaggaaagccaaagcacccaatggatatcctaaaacaacagggcaaagcaacatgggctacatcagttgagttacataaaaaatgggacaagtaaagatgtccagaaggaatccctgcagatatgaaacaagaggcaagtgcaaccgagggtcctcatgtacatttaaccacaattactggtcttggccagaccatgtattgttagtgcgggctaattatatgctgaatcagctagttaggaacacagataggactgacgggttatctctcatatcaggagcagggagagaagataggacacaggattttgtgctcggttctgccaatgttgtccagaattatatagaagggaatgcaaccataacaaaatctgcagcttgttatagcttgtacaacattatcaagcaacttcaagagaatgatgtgaaatctgcacgagacctgatggtagatgaccccaagcatgttgccctgcataaccttgtcttgtcctatatagacatgagtaagaatccagcaaatctcataaacagtctcaagagactacctaaagaaaaacttaaaaaattggccaaaatcataattcagctttctgctggttcagaaggagaaaatgccaatagtaataccctgcaaaagggtgacagcagtaattaagtgcaacacctcaggtgtgtgcctgtttaagaggacatatgagcataacatcattagccttggtgatctgatagaagaagtagcgaggatgattattatagatcacataaatagaaaacaatgcaatgaatgtagaaaagattttgaatttgtagcggttaacacatcttacacttagttacataaaaacaattgagcagctacccccaaagaaagatggggcaagtcaagatgacctccactgtcaaccttggatcaagcacatcctcaagatggactatagcaaagtcgcggtgcatgctgtgtcttcggactatgatgaattgtgctgttgttatatgtgcagtgctagttttgatcttccttgttgctacgataggcttgtctgtgaagttggctgtcacaataaaagaaaggaatacttgccagctgaggctatcagagctgagcacaactacagcatcaatcctgagatcaacaaatcagccatatccaggagggtcaacaagcacacccaaactgacaactgtcacatctattaccgatctcacccaccagtgtcctcaaaggaaagagttatgcaatggaacaataacatacaaaaactctgatggatgcctggatgaaaaagaaggagaatccattgactgtatagagctcatagccagatgtgtggaaactctatgcgaccccaaccctaactacaaccactgtatgtgcaccaagaacagcactgggctctggtgttgttacaattagagaaggggtaaacccatcatgcactcaaccactgcaaccagcatatgaatatatacatacaagtagattagttaattaaaatcagaagacgggacaagtatccagatggggtcagagctctacaccatagagggggtgagctcatctgaaatagtcctcaagcaagtcctcagaaggagcaaaagaatactgttaggactggtgttatcagccttaggcttgacgctcactagcactattgttatatctatttgtattagtgtagaacaggtcaaattacgacagtgtgtggacacttattgggcggaaaatggatccttacatccaggacagtcaacagaaaatactccaacaagagataagactacaacaaaagaccctagaagattacaggcgactggagcaggaaagtttgagagctgtgggtatgtgcaagttgttgatggtgatatgcatgatcacagttatgctgtactgggtggtgttgattgtttgggcttattggctctttgtgaatcaggaccaatttgtcagggagatacttggtctgaaggcggaaacttctgccgatgcactttttcttcccatggggtgagttgctgcaaaaaacccaaaagcgaggcaaccactgcccagaggaaccccaaaccagccaacagcaaatcaactcctccggcacattcagacagggccagcaaagaacataattcctcccaaggggaacaaccccgaagggagccaaccagcagcaatacaactattgctggcaccccatcaacagaggacactgctaagccaacgatcagcaaacctaaactcaccatcaggccctcgaaaagaggttcatccagcagcacaaaggcagcctccagaacccccaaccacaagaccaacaccagaggcaccagcaagacgaccgaccagagaccccgcaccggacccacccctgaaaggcccagacaaacccacagcacagcaaccccaccccccacaaccccaatccacaagggccgggccccaacccccaaaccaacaacagacctcaaggtcaacccaagggaaggcggcacaagcccaactgcaatacagaaaaacccaaccacacaaagtaatctcgtcgactgcacactgtctgatccaaatgagctaccaaggatttgttaccaggtaggaacttacaatcctagtcaatcaggaacctgcaacatagaggttccaaaatgttccacttatgggtatgcttgtatggctacattatatgacaccccattcaactgctggcgcagaaccaggagatgcatctgtgattccggaggggagctgattgagtggtgctgtactagtcaataaaaagtaacctttcacatctaaaataaagcaaaaagaagaaaaaagaagaaagaaggaaagaaagaagaagaatagcacacaacagaaaggagggaccaatatggacccatccaatgagcacacggtgaatgtgtatctgcctgactcctatttaaaaggagtcatctctttcagcgaaacaaatgcaattggatcttgcattataggtcgaccatacctaaaggatgactacactgctcatgttgcaaccacaagtcctgtcattgagcaccaaagattaaaaagcttgtttagtggtctaacaatcagcaagaactatcaatttgttgaacccctcatgatgcagaatgagctgctcaagatctctgccggtacaaaactcaagaagctaaagaaatggctaggtcgaagcaaagatattagtgagatcaagctgaagatggttgcagactggctcaaacttgcacatactcctgggaaagggaaagtgattgactgtatccaggtagacaacctacctgaatggctagagcaatggtttgattcatggttgatgcttaatgaactgatacaagcttataggtgcctggaagtgtcacagaccagtgctatcctccgtaaaagctgttggaatttcttctttgctatatcatcatttggctgcatcttgataagccggaaaagtaaaaggatttgtttctgcacctataaccagttcttgacatggaaagatttggcacttagcaggttcaatgctaacctgtgtgtctgggttagcaactgtttaaatagtactcaagaaggactaggactccggagcaaacttgttggagaactcctaaatcgattatatcatgagactgatgagttgttgagtggtactggcaatgagggattctggatagttaaggaattcgagggattcatcatgagtgaaattctgaggataactgaacatgcacagtttagtgtacgctttagaaacacactattaaatggaatcgtagaaaaaattgggaaaatgcgtgttgcccacagtcagcgaacttccaacactgccatagaaaatcatagatatcccggagagcaaagtttgttggagaaaacaggaagagtgctcaagattataaagctactagtccaaaacgatatgtccaatgcagcagagatgtattttatctttagaatattcggtcaccctatggtagaagaaagggaggccatggatgctgttcgggagaacagtgaggccactaaaatattgagcctcagggctctgacggagatgagaggagcttttatcctaagagtgatcaaaggttttgtaacaaattacaagaggtggccaagaataaagaacccttctacattgagtagcaggtggcgaatgtatatgagggctaaaacatatcctagtcaacttgagctatgtgctgaagacttcttagagttagcaggtatcagcttctgccaagaattctatgttcccaacaggaccagcctagaaatggtactcaatgacaaggcaatctcccctccaaagtctttgatctggtctgtatatccaaaaaattatttacctccttctgtacaggagcaatttcggttagaatctctggggaaagcagaacatgaaaaaactagacgtgtactagagttttacttgaaggatgccaactttaaccaagagaacttgaaaaagtatgtagtgttacagcactaccttaatgataaagagcatgttgtctctttgactggaaaagaaagggaattaagtgtggggaggatgtttgccatgcagccaggaaaacaaagacaagtgcagatacttgcagaaaaactgttatctgataatatagtaccatttttcccagaaactcttacaagatatggagacctggagttacaaagaattatggagcttaaatctgaactatcttcagtgaaagctaggaagagtgatagctacaacaattatatagcccgggcatcaatagtaacagatcttagcaagttcaaccaagcatttcggtatgaaacaacatcagtttgtgctgatgttgccgacgagctccatggtactcagagcctattttgctggttacatctaacagtttccgccaccactatgatatgcacttataggcatgcaccacctgacaccaaagggatttatgatattgactcaatccctgagcagagtgggttgtatagataccacatgggaggaattgaggggtggtgtcaaaaaatgtggaccatggaggcaatatctctccttgatgttgtgtcggttaagaacagggttcagctaacatcactcctaaatggtgacaatcagtccatagatgttagtaaacctgtgaggcttattggtactcagactgagatacaagcagactacagtttagcaattaaaatgttgactgcagtcagggatgcttattctgacataggacacaaattaaaagaaggtgaaacttatgtgtcaagggatctgcaattcatgagtaagactatacagtctgaaggggtcatgtacccagcctcaattaagaaagttctgagagttggcccatggataaacactatactagacgatatcaagacaagcatggaagccataggtagcctttgtcaggagttggagttccggggggaatcatttactacaagcttgataatacggaatttttggttatatgtccaatgttttgtagaagcaaagagacacagtttagcaggtagtcagatatctaatgaattaaatagaacacttaccaaagtaatgaaatttttccaagtaggcagtgagtcagacacagtaaatctttatatgaatataccgatgcagctgggaggcggagaccctgttgttgtgtataggagcttctataggcggacacctgacttcctaacggaagtactaactcatgtagagttgttactgacatcacttaatgttgatagaggtgtttacatgaggttcttcctcacactaatgaacacctgcaaaaacgacaatgcaacattaactacactcatgagggatcctcaggctattgggtcagaaaggcaggctaaaataacaagtgagatcaatcgtacagcagtcacaagtgtactaagcctggctcctaaccagctttttagtgatagtgcagtacattttagccagaatgaggaagaaattggtactgttatgcaagatgtagggcctgtataccctcatggcttaagggtcatatatgaagcattcccattccacaaagctgaaaaagtagttaacatgatagcaggtaccaaatctattacaaatatattgcaaaggacatccgccataagtggtttagacattgatagagcagttcatatgatgttgctcaatctaggactgttagggaggatattagagtcaggacctgtcacagacaccattgagttgcgtcacaacaataggatactttgttgtcaattatctaagcgcatacgggaaacatcatgggacggaatagaaattgtaggagtgtcatcccctagtatgttatcatgcttggatataaactatgtaacagcagcacagaggccaggagtattaatagagaagttctcggcagaaaagactacaagagggaagagagggcctaaggctccatgggttggatctagcacgcaggagaaaaaattaacagcagtgtacaataggcaagccttatcaaaagagcagagagatcagttagaaacaataggtaagattagatgggtgtacagaggggtaacaggactacggagacttttagatctagtctgcatgggaaccttaggacttccctacaagctgataaaacccctgttaccaagattcatgagtgtcaatttcctgcatcgtcttgcagtgagcagtagaccgatggagtttccttcatcagtcccagcataccgcacaactaatttccactttgacacaagccccatcaataaaagacttagtgagagatttggaaatgaggacattaacttggtcttccaaaatgctataagctgtgggattagtgtcatgtgtatagtggagcaattaacagggaagagcccaaagttagtcatgctagagccaattgtggaagatatagacataatgtctgctcctaatttccaagggagggtaaatttcaagagtgtgaagaaaattgtggctgatcagcacatcttcaaccctgatcatatcagcctcacaatgctggggagactcctactgcccactataagaagcaactcaaatgcagagagaattgcaactgagaatttctttaatgggaacaatatagtagaggtgctatccagttgccttgcatgccattggtgtacaattttgttgctcctcaccatggagaatagtattttccagaaagagtggggtgatgggtttgttacagaccatgcctttataaactttacatggttcctgatgagcttcaaaacatatttgctctgccactgggggggtgacgatgaaggagagctaaacattgtagaagacccaattgacaggctagctagaatagacaatagtttttggcgtatgatgagcaaggttttccttgaaccaaaagtcaagagacgattgatgctgtatgacaccacaatcctaaatgtctttggcagtatcagttttaaaaactggttcattgggaagcttaggtcagcagattatacagaaataccttggatagtaaatgcagaaggagacattgtggaacagaggcccgtcacggagtaccttaaaaccatggcagctgggactaatgtcaaagtgataatgctgagttactcagacatggcacatgctatgacacggctgcttagatgcaaaaacatgcaggacaatgttccgaccattaagaaggcagctaccccaaccgatgttacaccagctgtggacccaacaagagctttgttgttgtaccctaaagttacctttagcaaactgacaactttcaatgcggcatgtactagcctacgacaacccgggggaaacttgtccaaaaattacataactttgctgccttggcaccatgtgaaccgatacaatttcgtgcatagttctacagggtgcaaggtcagcataagaagctgtctcggtaaattggtggcaaaactagacctcagagttatttactttattggtgaaggtgcagggaacttgatgtcaaggacagcatgtgaatatcctggcttaaaatttgtctacaggagcctaaaagatgctaacgatcatcaccaccccacagaatatgtgcgtgtcatggggagcatcagcagaatagtggacttaggtgaggggttagctatggaagcgactgatgccacgaggagagagcattgggatttcatacacagagtgagcaaagaacccttgttactgacagtgtgtgatgctgaactcaaagaccctaataacgtacttttattaatcatacagtggagaaagcatgtgctttcgtgtaaaatatgcacatcatacgggacaagtgtgtacatgctgatcaaatatcatgcccagcaagagccaaaaaaactcccacattttgtacgggtggtttctatatacgttatgcagggtagtaaagtctccggttcagagtgttatctgctcattactctcggacatcagaacacagccccttgctatggagaggtgcgtgctgcaaagtcagtaatggctctcactgaagtctacaagaatcctataaggctagacaaaatagctgtcgaaatcaatctaaagtctttagccccaggattatctttaccaatcacagaggtgagcatgttgaagtacctttcaaacctaacacacttggacaactccaaaaatattagtgccaccattgggggcagcagagttgtagaaaagaaatgggcaatgtgcaaagttgcaaatgttgtaagatggctatgtcatgtcagtaggatgcccaaaggcgacctgaattatgatttctttgaagtaattgagagcacctatccggatatggtgaaattgctagacaaccttaactcttcagagttgaagaaactagtgaaagtgacagggtacatactgagcacaaaatagaccataaagaatgatcacacagtcgtacacagcaggtaagttgtaactaaaaatccaatagggaaaaggggtgcattagttataaaaaacaaaaaacaaaaattaaaaatgtattg

SEQ ID NO: 16 - RNA, 13474 nt, Avian metapneumovirus
  source 1..13474: mol_type = genomic RNA
tcaagtcacaatagaaaagaaaaatgggacaagtaaaaatgtctcttgaaagtattagactcagtgatctagagtacaagcatgcaatccttgatgagtcacaatatacaataaggagagatgttggtgctacaactgctatcacaccctctgaactacagccaaaggtatccaccctgtgtggaatgattttatttgctaagcatgcagattatgaaccagcagcagaagttggcatgcagtacatcagcactgcattaggagctgataagacacagcagatactcaaaagctctggcagtgaagtgcaaggggtcatgaccaagatagtgacactccctgcagaaggtcccatacgaaagcgggaagtacttaatatccatgacattgggcccgcatgggcagacaatgtagaacggactgcaagggagaccatgagcctaatggtaaaagagaaagctcaaatacccaaaaaccaaaagccgtcagctcttgatgcacctgtgatcttactatgtattggggcattaatattcactaagcttgcatccacagtagaggttgggctggagactgctattaggagagcctctcgggtgttaagtgatgcaatatcacgttaccctaggatggacataccacggattgccaaatccttctttgagctatttgaaaagaaagtgtactataggaaccttttcatagagtacggtaaggcacttgggagtacgtcttccgggagtaggatggagagcctctttgttaacatcttcatgcaagcttatggagctgggcagactatgctaagatggggtgttgtggcaagatcatccaataacatcatgttgggccatgtgtctgtgcaggcagagttaaggcaggtctcagaagtgtacgatcttgttaggaaaatgggtcctgaatcaggcctcctccacttgaggcaaagtccaaaagcaggcttactatcattaacaagttgccccaactttgcaagtgttgttttggggaatgcagctggcctaggcatcattgggatgtataagggcagagcacccaacctggagttgttttctgcagcagaaagttatgctagatccctcaaagagagcaacaagattaaccttgctgcccttgggctaactgaagatgagagagaggctgccacatcatacctaggcggagatgaagacaagtcacagaagtttgagtaattaaaaaaattcgggacaagtgaaaatgtctttccccgaaggcaaggatatcttgatgatgggaagtgaggcagctaagttggcagaggcttatcagcaatcaatcaagaattccacttctgtgagaagatccattagtggtgaccctgttagcacagtgtctgaaaaagttccattaccaccactatgtagctcagaaacttctagaggagcatgcataagacccacaaaatcaaccctgcctcccatcaaggaagtggagtccatctacccaaaactacccacagctccccctgatgccatgattgagaccgcccaccccgtaggggctccaaagaaggcacagaaaagggtgaaatttgagagttcaaaagcaggcaaatacaccaagctagaagaagaagccctggagctcctgtccgaccctgatgaagataatgatgagaaatcatctgtacttacatttgaggagaaggacaatgcaccatcatctatagaggcgaggttggaggctatagaggaaaaactcagcatgatattggggatgttaaagaccctgagcatagccaccgcagggccaaccgcagcgagagacggaatacgtgatgcaatggtgggagttagagaagagttgatcaatagcattatggcggaagctaaagggaagattgcagaaatgataaaggaagaagatgcacagagggcaaaaataggagatgggagtgtgaaactaacagagaaagctcgggaattaaacaggatgcttgaggatcaaagctcgagtggtgaatctgagacagaaagtgaagagactgaaccagatactgatggtgaaaatgatgatatatacagctttgatatgtagttatgaaaaaatgggacaagtaaacatggagtcctatattatagatacctatcaaggtgtgccctacacagctgctgttcaagtcgatctagttgagaaggacaacaaccctgcaaaactgacggtttggtttcctttatttcagtccagcactcctgctccagtgctgctggaccagctaaaaactctgtcaatcactacacaatatactgcttcccctgaaggaccagtgctacaagtaaatgctactgcacaaggtgcagccatgtcagctctgcccaagaagttttcagttagtgctgctgtagcacttgatgagtacagcaaacttgactttggggtactcacagtttgcgatgtaagggcagtttacctcaccactctgaagccatacggtatggtgtctaagattgtaacaaacatgaacaccgttgggcgcaaaactcacgacctgatagccctttgtgacttcattgacatggagagaggaataccagtaactatccctgcctacataaaggcggtatccatcaaagattctgagtcagctacagttgaagctgcgatcagtggtgaagctgaccaggctatcacccaggcaagaatagctccgtatgcagggttgattctggtcatgaccatgaacaaccccaaagggatattcaggaaactcggtgcaggaacacaagttattgtggagttagggccttatgtgcaggctgagagcttagggaaaatatgtaaaacatggaaccaccagagaaccaggtatgtcctgaagtcccgctaaaactggcaaacaaaagtctactattcttctgtagtttaataaaaatatggggcaagtaaaatgtacctcaaactgctactaataatttatttggtggttggggccagtgggaagatacaagaaacttacagtgaagaatcatgcagcactgtaaccaggggttacaaaagtgtgctcagaacgggttggtatacaaatgtgttcaacctagaaatagggaatgtggagaacataacatgtaatgatggtcctagccttatcagcactgaattatcactaactcagaatgccttgcaagagcttagaactgtttctgccgatcagattacaaaggagaatcgaatcctttcccataggaagaagaggtttgtgttgggtgcaattgcccttggagtggccaccacagctgctgtaacagccggcgtagctttagctaaaacaataagattagaaggggaggtgaaagccatcaagctagctttgcgcagtacaaatgaggctgtgtccacattaggcaatggcgttcgcatcttagcaacagctgttaatgacctaaaagaatttataagcaagaaattaacccctgcaataaaccaaaacaaatgcaacatagcagacataagaatggcaatcagctttggacagaacaacagaaggtttctaaatgtggtgagacaattttctgacagtgcaggaataacttccgcagtgtctttagatcttatgacagatgcagaattggttaaagccatcaaccgaatgccaacttcgtctggccagattagcctcatgctgaacaatagagcaatggttaggaggaaagggtttggaatacttataggtgtttacggggggactgtagtgtatatggtgcaactcccaatatttggagttatagaaacaccctgctggagagtggtagctgcacccctctgtagacatgagagggagagttatgcttgtctgctgcgggaagatcaggggtggtactgcactaatgcaggatcaactgcttactacccaaatgaagatgactgcgaggtaagagatgactatgtgttctgtgatactgcagcaggtataaatgttgcatcagaggtggagcagtgcaaccaaaacatatcaacctctacttacccttgcaaagttagcacagggagacaccctgtaagcatggtagccttaacccctttgggaggtttagtatcatgttatgaaggtgtgagttgctccattggcagcaacaaagtcggaatcattaaacaactcaacaaaggatgcacacacatacccaataatgaggcagacacaataactatagataacacaatttatcaactcagcaaggtggtgggtgagcaaaggactatcaaaggagctccggtggtaaataacttcaaccctctactgttccctgaggatcaatttaacgtagccttagatcaagtatttgagagtgttgataaatcaaaagacctgattgacaagtctaatgacttactggacatagaagttaagagtaatataggtgctgcattagccatcacaattttagtagtgcttagcatgtcaatcatagtgggcatagcttactatgtggttaaaaagaggaaagccaaagcacccaatggatatcctaaaacaacagggcaaagcaacatgggctacatcagttgagttacataaaaaatgggacaagtaaagatgtccagaaggaatccctgcagatatgaaacaagaggcaagtgcaaccgagggtcctcatgtacatttaaccacaattactggtcttggccagaccatgtattgttagtgcgggctaattatatgctgaatcagctagttaggaacacagataggactgacgggttatctctcatatcaggagcagggagagaagataggacacaggattttgtgctcggttctgccaatgttgtccagaattatatagaagggaatgcaaccataacaaaatctgcagcttgttatagcttgtacaacattatcaagcaacttcaagagaatgatgtgaaatctgcacgagacctgatggtagatgaccccaagcatgttgccctgcataaccttgtcttgtcctatatagacatgagtaagaatccagcaaatctcataaacagtctcaagagactacctaaagaaaaacttaaaaaattggccaaaatcataattcagctttctgctggttcagaaggagaaaatgccaatagtaataccctgcaaaagggtgacagcagtaattaagtgcaacacctcaggtgtgtgcctgtttaagaggacatatgagcataacatcattagccttggtgatctgatagaaggagtagcgaggatgattattatagatcacataaatagaaaacaatgcaatgaatgtagaaaagattttgaatttgtagcggtttacacatcttacacttagttacataaaaacaattgagcagctacccccaaagaaagatggggcaagtcaagatgacctccactgtcaaccttggatcaagcacatcctcaagatggactatagcaaagtcgcggtgcatgctgtgtcttcggactatgatgaattgtgctgttgttatatgtgcagtgctagttttgatcttccttgttgctacgataggcttgtctgtgaagttggctgtcacaataaaagaaaggaatacttgccagctgaggctatcagagctgagcacaactacagcatcaatcctgagatcaacaaatcagccatatccaggagggtcaacaagcacacccaaactgacaactgtcacatctattaccgatctcacccaccagtgtcctcaaaggaaagagttatgcaatggaacaataacatacaaaaactctgatggatgcctggatgaaaaagaaggagaatccattgactgtatagagctcatagccagatgtgtggaaactctatgcgaccccaaccctaactacaaccactgtatgtgcaccaagaacagcactgggctctggtgttgttacaattagagaaggggtaaacccatcatgcactcaaccactgcaaccagcatatgaatatatacatacaagtagattagttaattaaaatcagaagacgggacaagtatccagatggggtcagagctctacaccatagagggggtgagctcatctgaaatagtcctcaagcaagtcctcagaaggagcaaaagaatactgttaggactggtgttatcagccttaggcttgacgctcactagcactattgttatatctatttgtattagtgtagaacaggtcaaattacgacagtgtgtggacacttattgggcggaaaatggatccttacatccaggacagtcaacagaaaatactccaacaagagataagactacaacaaaagaccctagaagattacaggcgactggagcaggaaagtttgagagctgtgggtatgtgcaagttgttgatggtgatatgcatgatcacagttatgctgtactgggtggtgttgattgtttgggcttattggctctttgtgaatcaggaccaatttgtcagggagatacttggtctgaaggcggaaacttctgccgatgcactttttcttcccatggggtgagttgctgcaaaaaacccaaaagcgaggcaaccactgcccagaggaaccccaaaccagccaacagcaaatcaactcctccggcacattcagacagggccagcaaagaacataattcctcccaaggggaacaaccccgaagggagccaaccagcagcaatacaactattgctggcaccccatcaacagaggacactgctaagccaacgatcagcaaacctaaactcaccatcaggccctcgaaaagaggttcatccagcagcacaaaggcagcctccagaacccccaaccacaagaccaacaccagaggcaccagcaagacgaccgaccagagaccccgcaccggacccacccctgaaaggcccagacaaacccacagcacagcaaccccaccccccacaaccccaatccacaagggccgggccccaacccccaaaccaacaacagacctcaaggtcaacccaagggaaggcggcacaagcccaactgcaatacagaaaaacccaaccacacaaagtaatctcgtcgactgcacactgtctgatccaaatgagccaccaaggatttgttaccaggtaggaacttacaatcctagtcaatcaggaacctgcaacatagaggttccaaaatgttccacttatgggtatgcttgtatggctacattatatgacaccccattcaactgctggcgcagaaccaggagatgcatctgtgattccggaggggagctgattgagtggtgctgtactagtcaataaaaagtaacctttcacatctaaaataaagcaaaaagaagaaaaaagaagaaagaaggaaagaaagaagaagaatagcacacaacagaaaggagggaccaatatggacccatccaatgagcacacggtgaatgtgtatctgcctgactcctatttaaaaggagtcatctctttcagcgaaacaaatgcaattggatcttgcattataggtcgaccatacctaaaggatgactacactgctcatgttgcaaccacaagtcctgtcattgagcaccaaagattaaaaagcttgtttagtggtctaacaatcagcaagaactatcaatttgttgaacccctcatgatgcagaatgagctgctcaagatctctgccggtacaaaactcaagaagctaaagaaatggctaggtcgaagcaaagatattagtgagatcaagctgaagatggttgcagactggctcaaacttgcacatactcctgggaaagggaaagtgattgactgtatccaggtagacaacctacctgaatggctagagcaatggtttgattcatggttgatgcttaatgaactgatacaagcttataggtgcctggaagtgtcacagaccagtgctatcctccgtaaaagctgttggaatttcttctttgctatatcatcatttggctgcatcttgataagccggaaaagtaaaaggatttgtttctgcacctataaccagttcttgacatggaaagatttggcacttagcaggttcaatgctaacctgtgtgtctgggttagcaactgtttaaatagtactcaagaaggactaggactccggagcaaacttgttggagaactcctaaatcgattatatcatgagactgatgagttgttgagtggtactggcaatgagggattctggatagttaaggaattcgagggattcatcatgagtgaaattctgaggataactgaacatgcacagtttagtgtacgctttagaaacacactattaaatggaatcgtagaaaaaattgggaaaatgcgtgttgcccacagtcagcgaacttccaacactgccatagaaaatcatagatatcccggagagcaaagtttgttggagaaaacaggaagagtgctcaagattataaagctactagtccaaaacgatatgtccaatgcagcagagatgtattttatctttagaatattcggtcaccctatggtagaagaaagggaggccatggatgctgttcgggagaacagtgaggccactaaaatattgagcctcagggctctgacggagatgagaggagcttttatcctaagagtgatcaaaggttttgtaacaaattacaagaggtggccaagaataaagaacccttctacattgagtagcaggtggcgaatgtatatgagggctaaaacatatcctagtcaacttgagctatgtgctgaagacttcttagagttagcaggtatcagcttctgccaagaattctatgttcccaacaggaccagcctagaaatggtactcaatgacaaggcaatctcccctccaaagtctttgatctggtctgtatatccaaaaaattatttacctccttctgtacaggagcaatttcggttagaatctctggggaaagcagaacatgaaaaaactagacgtgtactagagttttacttgaaggatgccaactttaaccaagagaacttgaaaaagtatgtagtgttacagcactaccttaatgataaagagcatgttgtctctttgactggaaaagaaagggaattaagtgtggggaggatgtttgccatgcagccaggaaaacaaagacaagtgcagatacttgcagaaaaactgttatctgataatatagtaccatttttcccagaaactcttacaagatatggagacctggagttacaaagaattatggagcttaaatctgaactatcttcagtgaaagctaggaagagtgatagctacaacaattatatagcccgggcatcaatagtaacagatcttagcaagttcaaccaagcatttcggtatgaaacaacatcagtttgtgctgatgttgccgacgagctccatggtactcagagcctattttgctggttacatctaacagtttccgccaccactatgatatgcacttataggcatgcaccacctgacaccaaagggatttatgatattgactcaatccctgagcagagtgggttgtatagataccacatgggaggaattgaggggtggtgtcaaaaaatgtggaccatggaggcaatatctctccttgatgttgtgtcggttaagaacagggttcagctaacatcactcctaaatggtgacaatcagtccatagatgttagtaaacctgtgaggcttattggtactcagactgagatacaagcagactacagtttagcaattaaaatgttgactgcagtcagggatgcttattctgacataggacacaaattaaaagaaggtgaaacttatgtgtcaagggatctgcaattcatgagtaagactatacagtctgaaggggtcatgtacccagcctcaattaagaaagttctgagagttggcccatggataaacactatactagacgatatcaagacaagcatggaagccataggtagcctttgtcaggagttggagttccggggggaatcatttactacaagcttgataatacggaatttttggttatatgtccaatgttttgtagaagcaaagagacacagtttagcaggtagtcagatatctaatgaattaaatagaacacttaccaaagtaatgaaatttttccaagtaggcagtgagtcagacacagtaaatctttatatgaatataccgatgcagctgggaggcggagaccctgttgttgtgtataggagcttctataggcggacacctgacttcctaacggaagtactaactcatgtagagttgttactgacatcacttaatgttgatagaggtgtttacatgaggttcttcctcacactaatgaacacctgcaaaaacgacaatgcaacattaactacactcatgagggatcctcaggctattgggtcagaaaggcaggctaaaataacaagtgagatcaatcgtacagcagtcacaagtgtactaagcctggctcctaaccagctttttagtgatagtgcagtacattttagccagaatgaggaagaaattggtactgttatgcaagatgtagggcctgtataccctcatggcttaagggtcatatatgaagcattcccattccacaaagctgaaaaagtagttaacatgatagcaggtaccaaatctattacaaatatattgcaaaggacatccgccataagtggtttagacattgatagagcagttcatatgatgttgctcaatctaggactgttagggaggatattagagtcaggacctgtcacagacaccattgagttgcgtcacaacaataggatactttgttgtcaattatctaagcgcatacgggaaacatcatgggacggaatagaaattgtaggagtgtcatcccctagtatgttatcatgcttggatataaactatgtaacagcagcacagaggccaggagtattaatagagaagttctcggcagaaaagactacaagagggaagagagggcctaaggctccatgggttggatctagcacgcaggagaaaaaattaacagcagtgtacaataggcaagccttatcaaaagagcagagagatcagttagaaacaataggtaagattagatgggtgtacagaggggtaacaggactacggagacttttagatctagtctgcatgggaaccttaggacttccctacaagctgataaaacccctgttaccaagattcatgagtgtcaatttcctgcatcgtcttgcagtgagcagtagaccgatggagtttccttcatcagtcccagcataccgcacaactaatttccactttgacacaagccccatcaataaaagacttagtgagagatttggaaatgaggacattaacttggtcttccaaaatgctataagctgtgggattagtgtcatgtgtatagtggagcaattaacagggaagagcccaaagttagtcatgctagagccaattgtggaagatatagacataatgtctgctcctaatttccaagggagggtaaatttcaagagtgtgaagaaaattgtggctgatcagcacatcttcaaccctgatcatatcagcctcacaatgctggggagactcctactgcccactataagaagcaactcaaatgcagagagaattgcaactgagaatttctttaatgggaacaatatagtagaggtgctatccagttgccttgcatgccattggtgtacaattttgttgctcctcaccatggagaatagtattttccagaaagagtggggtgatgggtttgttacagaccatgcctttataaactttacatggttcctgatgagcttcaaaacatatttgctctgccactgggggggtgacgatgaaggagagctaaacattgtagaagacccaattgacaggctagctagaatagacaatagtttttggcgtatgatgagcaaggttttccttgaaccaaaagtcaagagacgattgatgctgtatgacaccacaatcctaaatgtctttggcagtatcagttttaaaaactggttcattgggaagcttaggtcagcagattatacagaaataccttggatagtaaatgcagaaggagacattgtggaacagaggcccgtcacggagtaccttaaaaccatggcagctgggactaatgtcaaagtgataatgctgagttactcagacatggcacatgctatgacacggctgcttagatgcaaaaacatgcaggacaatgttccgaccattaagaaggcagctaccccaaccgatgttacaccagctgtggacccaacaagagctttgttgttgtaccctaaagttacctttagcaaactgacaactttcaatgcggcatgtactagcctacgacaacccgggggaaacttgtccaaaaattacataactttgctgccttggcaccatgtgaaccgatacaatttcgtgcatagttctacagggtgcaaggtcagcataagaagctgtctcggtaaattggtggcaaaactagacctcagagttatttactttattggtgaaggtgcagggaacttgatgtcaaggacagcatgtgaatatcctggcttaaaatttgtctacaggagcctaaaagatgctaacgatcatcaccaccccacagaatatgtgcgtgtcatggggagcatcagcagaatagtggacttaggtgaggggttagctatggaagcgactgatgccacgaggagagagcattgggatttcatacacagagtgagcaaagaacccttgttactgacagtgtgtgatgctgaactcaaagaccctaataacgtacttttattaatcatacagtggagaaagcatgtgctttcgtgtaaaatatgcacatcatacgggacaagtgtgtacatgctgatcaaatatcatgcccagcaagagccaaaaaaactcccacattttgtacgggtggtttctatatacgttatgcagggtagtaaagtctccggttcagagtgttatctgctcattactctcggacatcagaacacagccccttgctatggagaggtgcgtgctgcaaagtcagtaatggctctcactgaagtctacaagaatcctataaggctagacaaaatagctgtcgaaatcaatctaaagtctttagccccaggattatctttaccaatcacagaggtgagcatgttgaagtacctttcaaacctaacacacttggacaactccaaaaatattagtgccaccattgggggcagcagagttgtagaaaagaaatgggcaatgtgcaaagttgcaaatgttgtaagatggctatgtcatgtcagtaggatgcccaaaggcgacctgaattatgatttctttgaagtaattgagagcacctatccggatatggtgaaattgctagacaaccttaactcttcagagttgaagaaactagtgaaagtgacagggtacatactgagcacaaaatagaccataaagaatgatcacacagtcgtacacagcaggtaagttgtaactaaaaatccaatagggaaaaggggtgcattagttataaaaaacaaaaaacaaaaattaaaaatgtattg

SEQ ID NO: 17 - protein, 278 aa, Avian metapneumovirus
MSFPEGKDILMMGSEAAKMADAYQRSLRNTSAGGRSISGEPINTIAEKVPLPPLCNPTTPRGSCIKPNKAPVSKVKEIESIYPKLPTAPVATDTETSTSTESAKKSKKVKFDNPKVGKYTKLEEEALELLSDPEEDNDEKSSILTFEEKDTASTSIEARLEAIEEKLSMILGMLKTLNIATAGPTAARDGIRDAMIGMREELINSIMTEAKDKIAEMMKEEDTQRAKIGDGSVKLTEKAKELNKVLEDQSSSGESESEEESGESDFNEEESDIYNLDL

SEQ ID NO: 18 - protein, 254 aa, Avian metapneumovirus
MESYIIDTYQGVPYTAAVQVDLIEKDSNPATLTVWFPLFQSSTPAPVLLDQLKTLSITTQYTASPEGPVLQVNAAAQGAAMSALPKKFAVSAAVALDEYSRLEFGTLTVCDVRSIYLTTLKPYGMVSKIMTDVRSVGRKTHDLIALCDFIDIEKGVPITIPAYIKAVSIKDSESATVEAAISGEADQAITQARIAPYAGLILIMTMNNPKGIFKKLGAGMQVIVELGPYVQAESLGKICKTWNHQRTRYVLRSR

SEQ ID NO: 19 - protein, 538 aa, Avian metapneumovirus
MDVRICLLLFLISNPGSCIQETYNEESCSTVTRGYKSVLRTGWYTNVFNLEIGSVENITCNDGPSLIDTELVLTKNALRELKTVSADQVAKESRLSSPRRRRFVLGAIALGVATAAAVTAGVALAKTIRLEGEVKAIKNALRNTNEAVSTLGNGVRVLATAVNDLKEFISKKLTPAINRNKCNIADIKMAISFGQNNRRFLNVVRQFSDSAGITSAVSLDLMTDDELVRAINKMPTSSGQISLMLNNRSMVRRKGFGILIGVYDGTVVYMVQLPIFGVIETPCWRVVAAPLCREEKGNYACILREDQGWYCTNAGSTAYYPNEDDCEVRDDYVFCDTAAGINVASEVEQCNYNISTSKYPCKVSTGRHPVSMVALTPLGGLVSCYEGVSCSIGSNKVGIIKQLGKGCTHIPNNEADTITIDNTVYQLSKVVGEQRTIKGAPVVNNFNPILFPEDQFNVALDQVFESIDRSQDLIDKSNDLLGADAKSKAGIAIAIVVLVILGIFFVLAVIYYCFRVQKTKPKHDYPATTGHSSMAYVS

SEQ ID NO: 20 - protein, 73 aa, Avian metapneumovirus
MPIVIPCKRVTAVIKCNTSGVCLFKRTYEHNIISLGDLIEDVARMIIIDHINRKQCNECRKDFEFVAVYTSYT

SEQ ID NO: 21 - protein, 414 aa, Avian metapneumovirus
MGSELYTIEGVSSSEIVLKQVLRRSKRILLGLVLSALGLTLTSTIVISICISVEQVKLRQCVDTYWAENGSLHPGQSTENTPTRDKTATKDPRRLQATGAGKFESCGYVQVVDGDMHDHSYAVLGGVDCLGLLALCESGPICQGDTWSEGGNFCRCTFSSHGVSCCKKPKSEATTAQRNPKPANSKSTPPVHSDRASKEHNSSQGEQPRREPTSSNTTIAGTPSTEDTAKPTISKPKLTIRPSKRGSSSSTKAASRTPNHKTNTRGTSKTTDQRLRTGPTPERPRQTHSTATPPPTTPIHKGRAPTPKPTTDLKVNPREGGISPTAIQKNPTTQSNLVDCTLSDPNEPPRICYQVGTYNPSQSGTCNIEVPKCSTYGYACMATLYDTPFNCWRRTRRCICDSGGELIEWCCTSQ

SEQ ID NO: 22 - protein, 2004 aa, Avian metapneumovirus
MEISDESVVNVYLPDSYLKGVISFSETNAIGSCVLNRPYIKDDHTAHVAMTNPVIEHQRLRALFKSLTISREYRVVEPLMIQKELLKVAAGARLKKLKKWLGRSKDISEVKLKMVTDWLKLSQTPGRGKIIDCIQVENLPDWLEHWFDSWLILNDVIQSYRCLEVSQTSAILRKSSLNFFFAVSSFGCIIISRKSRRICFCTYNQLLTWKDLALSRFNANLCVWVSNCLNSAQDGLGLRSKLVGELLNRLYCETDELLSITGNEGYGIVKEFEGFIMSEILRMTEHAQFSVRFRNTLLNGIADKIGKMRAAYQRRTQNTSVEHHRYPSEHEMLEATGRVLRIIKLLVNNEIANAAEMYFIFRIFGHPMVEEREAMDAVRDNSETAKILSLVALTEMRGAFILRVIKGFVGNYKRWPKIKNPSTLSRRWKMYMNAKTYPSNLELCVEDFLELAGISFCQEFYVPSQTSLEMVLNDKAISPPKSLIWSVYPRNYLPQNIQDRFRLESLDRLEHERTRRVLEFYLKDANFKQSDLKKYVVGQEYLNDKEHVVSLTGKERELSVGRMFAMQPGKQRQVQILAEKLLSDNIVPFFPETLTRYGDLELQRIMELKSELSSTKKRKSDSYNNYIARASIVTDLSKFNQAFRYETTSVCADVADELHGTQSLFCWLHLTVSSTTMICTYRHAPPDTGGIYDIDQIPEQSGLYRFHMGGIEGWCQKMWTMEAISLLDVVSVRNRVQLTSLLNGDNQSIDVSKPVRLTGAQTEIQADYSLAIKMLTAVRDAYYNIGHKLKEGETYVSRDLQFMSKTIQSEGVMYPASIKKVLRVGPWINTILDDIKTSMEAIGSLCQELEFRGESFVTSLIIRNFWLYIQCFVEAKNHSLAGAQISNELNRTLTKVKRFFRLADESSTVNLFMNIPMQLGGGDPVVVYRSFYRRTPDFLTEVLTHMELLLTAIEMDRGVYMNFFLTLLNTCKNDRATLTTLMRDPQAIGSERQAKVTSEINRTAVTSVLSLAPNPLFSDSAVHFSQNEEEIGTVMEDISPVYPHGLRVVYEAFPFHKAEKVVNMIAGTKSITNILQRTSAISGVDIDRAVHMMLLNLGLLGRMLESGPITDTIDLRSNNRILCCQLSKRIREMSWDGLEIVGVSSPSMLSSLDINYVTIAQKPGIIVEKFSAEKTTRGKRGPKAPWVGSSTQEKKLVAVYNRQVLSKEQKDQLETIGKIRWVYKGVTGLRRLLNLICLGTLGLPYKLLKPLLPRFMSVNFLHRLAVSSRPMEFPSSVPAYRTTNFHFDTSPINKRLSERFGNEDINLVFQNAISCGISVMCVVEQLTGRSPKQVILEPIVEDIDIMSAPNFQGRLNFKSVRKIVTDQHIFNPDHIDLVMLGKLLLPTVRSNINNNKLATENFFNGNNIVEALTSCLACHWCTVLILLTTENSIFQKEWGDGFITDHAFINFTWFLMSFKTYLLCHWQTEKNKTLGIVEDPIDRLIRVDNSFWRMMSKVFLEPKVKRRLMLYDTSVLNVLGSISFKNWFVSRLRTVDYMEIPWIVNAEGDIVEQKPVNEYLKIMAAGVHVKVIMLSYSDMAHAMTRLLRCKNMQDNVPTIKKTVSPTDVTPSVDPTRALLLFPKVIFSKLTTFNATCSTAKQSGSNLTKNYMTLMPWHHINRYNFVHSSTGCKVSVKGCIGKLITKLELRIIYFIGEGAGNLMSRTACEYPNLKFVYRSLKDANDHHHPTEYVRVMGSISRIIDLGEGLAMEATDATKREHWDFIHRVCKDPLLLTICDAELKNMEDVLHLVIQWRKHVLSCNICTSYGTNVYMFVKYHAQSEAKKLPHFVRVVSIYIMQGSKLSGSECYMLLTLGHQNMAPCYGEVRSAKSIMALTSTFKHPCRLDKSAIEANLKSLAPGLTLPLSEPAMQKYLLDLSSLSGIKNTGATIGGSKVVEKKWALCKVSNIVRWLSHVLKMPKGDLNYDFFEVIENTYPDMVKLLDNLTPSELKKLVKITGYILGTK